(19) United States
(12) Patent Application Publication
(10) Pub. No.: US 2022/0059790 A1
(43) Pub. Date: Feb. 24, 2022

(54) LIGHT-EMITTING MEMBER, BIOLOGICAL AUTHENTICATION DEVICE, WRIST-BAND TYPE ELECTRONIC APPARATUS, AND BIOLOGICAL MEASUREMENT DEVICE

(71) Applicant: Konica Minolta, Inc., Tokyo (JP)

(72) Inventors: Mio WATANABE, Hino-shi, Tokyo (JP); Ryo NAKABAYASHI, Hachsoji-shi,. Tokyo (JP); Kenichi TABATA, Inagi-shi, Tokyo (JP); Rie KATAKURA, Hino-shi, Tokyo (JP); Yasuo MIYATA, Yokohama-shl, Kanagawa (JP)

(21) Appl. No.: 17/275,890

(22) PCT Filed: Sep. 12, 2019
(86) PCT No.: PCT/JP2019/035924

§ 371 Date: Mar. 12, 2021

(30) Foreign Application Priority Data
Sep. 14, 2018 (JP) 2018-172416
Feb. 5, 2019 (JP) 2019-031349

(51) Int. Cl. H01L 51/50; H01L 51/00

(52) U.S. Cl. CPC H01L 51/5036; H01L 51/0071; H01L 51/0091; H01L 51/0059

(57) ABSTRACT

Provided is a light emitting member that emits near-infrared light, containing a surface light source that emits at least red light and a wavelength conversion film that converts visible light of the surface light source into near-infrared light, wherein the light emitting member has a maximum emission wavelength in the wavelength range of 700 to 1500 nm.

### TECHNICAL FIELD

The present invention relates to a light emitting member, a biometric authentication device, a wristband type electronic device, and a biometric device. More specifically, the present invention relates to a light emitting member having a maximum emission wavelength in the near-infrared light region and having excellent emission intensity and emission life, a biometric authentication device, a wristband type electronic device, and a biometric device equipped with the same.

### BACKGROUND

At present, organic electroluminescent elements, which are self-luminous all-solid-state light emitting devices that emit uniform light over the entire surface, are being actively developed. Organic EL elements used for lighting and displays can emit light in any color depending on their configuration, and those that emit invisible light such as near-infrared light in addition to visible light are also known. Near-infrared light is used in various analytical instruments and is rapidly attracting attention in the information-oriented society. It is also used for biometric authentication using biometric data (for example, fingerprints, veins, irises, retinas, palms, facial features, body shapes, and voiceprints) possessed by a person. Since biometric authentication uses a part of the living body and the characteristics of its operation (biometric information), it has higher security than a key or password, and is characterized by not being forgotten or lost. Therefore, as the importance of security measures in bank ATMs (Automated Teller Machines), mobile phones, PDAs (Personal Data Assistants), and personal computers increases, biometrics are extremely useful as a technology aimed at preventing unauthorized access and leakage of corporate and personal information.

Among biometrics, the authentication method using fingerprints (fingerprint authentication) has been put into practical use as entry/exit of public facilities and login authentication of electronic devices because it is relatively low cost and can be miniaturized. The fingerprint authentication method is the oldest and most proven biometric authentication method, and a fingerprint input device using a total internal reflection prism has been put into practical use for a long time. However, since it is difficult to miniaturize this method, it is not suitable for mobile terminals such as notebook computers, PDAs, and mobile phones. In addition, the fingerprint authentication method may be difficult to authenticate depending on the skin type of the individual, and may be unclean in terms of hygiene. Therefore, in recent years, a vein authentication which can achieve non-contact authentication and is more difficult to duplicate and enables advanced security measures, is attracting attention.

The principle of vein recognition is to utilize the property that hemoglobin of red blood cells flowing through veins absorbs light in the near-infrared light region, and authentication is performed using a finger vein image taken with near-infrared light.

In the above vein authentication, it is preferable to use a surface light source that emits light uniformly on the light emitting surface and has a maximum emission in the near-infrared light region from the viewpoint of improving the authentication accuracy. As such a surface light source, an organic electroluminescent element that emits light in a near-infrared light region of 700 nm or more is disclosed. In this organic electroluminescent element, an organic functional layer including an anode, a cathode, and a light emitting layer sandwiched between them is arranged, and the light emitting layer contains a host material, a delayed phosphor, and a light emitter (for example, refer to Patent Document 1).

However, the organic electroluminescent element disclosed in Patent Document 1 has a configuration in which a delayed phosphor and a light emitter are contained in a light emitting layer, and when applied to a biometric authentication device, the light emitting intensity and the light emitting life are inferior. In particular, among the organic electroluminescent elements disclosed in Patent Document 1, none of them has both light emission in a wavelength range exceeding 700 nm, which has a specifically high transmittance of living tissue and called as “biological (spectroscopic) window”, and a long life.

### PRIOR ART DOCUMENTS

### Patent Document

Patent Document 1: JP-A 2018-92993

### SUMMARY OF THE INVENTION

### Problems to be Solved by the Invention

The present invention has been made in view of the above problems and situations. An object of the present invention is to provide a light emitting member having a maximum emission wavelength in the near-infrared light region and having excellent emission intensity and emission life, and a biometric authentication device, a wristband type electronic device, and a biometric measurement device equipped with the light emitting member.

### Means to Solve the Problems

As a result of examining the causes of the above problems in order to solve the above problems, the present inventors have found the following. That is, by independently providing a surface light source that emits red light and a wavelength conversion film having a function of converting visible light of the surface light source into near-infrared light, it has been found that a light emitting member having a maximum emission wavelength in the near-infrared light region and having excellent emission intensity and emission life was realized, and the present invention has been made.

That is, the above problem according to the present invention is solved by the following means.

1. A light emitting member that emits near-infrared light, comprising a surface light source that emits at least red light and a wavelength conversion film that converts visible light of the surface light source into near-infrared light, wherein the light emitting member has a maximum emission wavelength in the wavelength range of 700 to 1500 nm.
2. The light emitting member according to item 1, wherein the wavelength conversion film contains a luminescent dye.
3. The light emitting member according to item 2, wherein the luminescent dye is a squarylium compound, a croconium compound, or a thiadiazole compound.
4. The light emitting member according to item 3, wherein the squarylium compound is a compound having a structure represented by the following Formula (1).

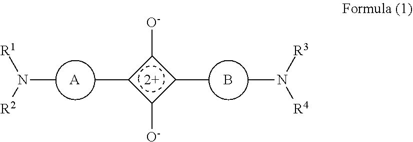

In the formula, A and B respectively represent an aromatic hydrocarbon ring which may have a substituent, or an aromatic heterocycle which may have a substituent, and adjacent substituents may be bonded to each other to form a ring. R¹ to R⁴ respectively represent a substituent, and at least one of R¹ to R⁴ has an aromatic hydrocarbon ring. Further, R¹ to R⁴ may be respectively bonded to each other to form a ring.

5. The light emitting member according to item 4, wherein the squarylium compound having a structure represented by Formula (1) is a compound having a structure represented by the following Formula (2).

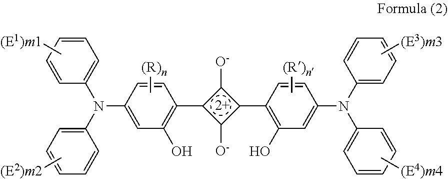

In the formula, E¹ to E⁴ respectively represent a substituent. m1 to m4 respectively represent 0 or an integer of 1 to 5. R and R′ respectively represent a substituent and n and n′ respectively represent 0 or an integer of 1 to 3, provided that when m1 to m4, n and n′ are 2 or more, E¹ to E⁴, R and R′ may be the same or different.

6. The light emitting member according to any one of items 2 to 5, wherein the wavelength conversion film contains two or more luminescent dyes.
7. The light emitting member according to any one of items 2 to 6, wherein the wavelength conversion film contains two or more squarylium dyes.
8. The light emitting member according to any one of items 1 to 7, wherein the surface light source is an organic electroluminescent element.
9. The light emitting member according to any one of items 1 to 8, wherein the maximum emission wavelength obtained from the light emitting member is 780 nm or more.
10. A biometric authentication device equipped with the light emitting member according to any one of items 1 to 9.
11. A wristband electronic device equipped with the biometric authentication device according to item 10 so as to carry out biometric authentication by taking an image of a wrist vein.
12. The wristband electronic device according to item 11, wherein when the wristband electronic device is mounted to a wrist, a light source is provided in any area on a wristband of the wristband electronic device other than on the same plane as an imaging unit.
13. A biometric device equipped with the light emitting member according to any one of items 1 to 9.
14. The biometric device according to item 13, being a pulse oximeter that performs measurement at a wrist or a base of a finger.
15. The biometric device according to item 13, being a pulse wave sensor that performs measurement at a wrist or a base of a finger.

### Effects of the Invention

By the above means of the present invention, it is possible to provide a light emitting member having a maximum emission wavelength in the near-infrared light region and having excellent emission intensity and emission life. It is also possible to provide a biometric authentication device, a wristband type electronic device, and a biometric device equipped with the light emitting member.

The expression mechanism and the action mechanism that enable to solve the above-mentioned problems by setting the production conditions specified in the present invention is speculated as follows.

As described above, by utilizing the property of absorbing light in the near-infrared region of hemoglobin of erythrocytes flowing through veins, it has been proposed a method in which the light emitting layer of the organic EL element contains a host material, a delayed phosphor and a luminescent dye, and emits light in the near-infrared light of wavelength of 700 nm or more. However, in the method in which the luminescent dye coexists in the light emitting layer of the organic EL element in this way, when the organic EL element is driven, the luminescent dye added to the light emitting layer acts as a quenching material, so that the light emitting life is presumed to be extremely short. Based on the above problems, as defined in the present invention, by forming the surface light source and the wavelength conversion film containing the luminescent dye in independent forms, the luminescent dye contained in the wavelength conversion film does not affect the light emission of the separated surface light sources. It was possible to obtain a light emitting member having a long life, which has a remarkable effect on the light emitting life.

More specifically, a surface light source that emits light in the visible light region and a wavelength conversion film that converts visible light from the surface light source into light emission in the near-infrared light region are allowed to exist individually in independent forms. It is assumed that the material having wavelength conversion ability (light emitter) is less likely to be deteriorated by current excitation during light emission of the surface light source, and the energy transfer between the red light emitter in the surface light source is not hindered by the light emitter having wavelength conversion ability.

On the other hand, as a point light source used for biometric authentication, a point light source represented by a light emitting diode (LED) is currently used, but these LEDs have variations in the amount of light depending on the product. Therefore, when a plurality of LEDs are used as light sources, the brightness uniformity is about 50% or less, and a complicated program for adjusting and controlling the amount of light is required. Further, there is a difference in brightness in the obtained image, and when it is applied as a light source for biometric authentication without processing, the uneven portion (unevenness) of the brightness of the light source is erroneously recognized as an image of the object to be measured. Therefore, it often causes a decrease in the authentication rate.

The present inventions have diligently studied the above problems and found the following. A new light emitting member is applied as a surface light source for biometric authentication device. This light emitting member is provided with at least a surface light source that emits red light, for example, an organic EL element, and a wavelength conversion film that converts visible light of the surface light source into near-infrared light, and this light emitting member has a maximum emission wavelength in the wavelength range of 700 to 1500 nm. As a result, it was possible to obtain a light emitting member having a maximum emission wavelength in the near-infrared light region exceeding 700 nm, which is preferable for vein recognition, and having excellent emission uniformity, emission intensity, and emission life.

It should be noted that each of the above technical mechanisms is only speculation and does not limit the technical scope of the present invention.

### BRIEF DESCRIPTION OF THE DRAWINGS

FIG. 1 is a schematic cross-sectional view showing an example of a configuration of a light emitting member of the present invention.

FIG. 2 is a schematic cross-sectional view showing an example of a configuration of a wavelength conversion film provided with a sealing structure according to the present invention.

FIG. 3A is a schematic diagram showing an example of transmissive sensing at a fingertip using a point light source.

FIG. 3B is a schematic diagram showing an example of transmissive sensing at a fingertip using a surface light source.

FIG. 3C is a schematic diagram showing an example of reflective sensing at a fingertip using a surface light source.

FIG. 4A is a schematic showing an example of transmissive sensing at a base of a finger using a point light source.

FIG. 4B is a schematic diagram showing an example of transmissive sensing at a base of a finger using a surface light source.

FIG. 5A is a schematic diagram showing an example of V-shaped sensing on a wrist using a point light source.

FIG. 5B is a schematic diagram showing an example of V-shaped sensing on a wrist using a surface light source.

FIG. 5C is a schematic diagram showing an example of reflective sensing on a wrist using a surface light source.

FIG. 5D is a schematic diagram showing an example of reflective sensing on a wrist using a point light source.

FIG. 6 is a perspective view showing an example of a configuration of a wristband type electronic device.

FIG. 7 is a chart showing an example of measurement results of a transmissive pulse wave sensor at a fingertip.

FIG. 8 is a chart showing an example of measurement results of a reflective pulse wave sensor at a fingertip.

FIG. 9 is a chart showing an example of measurement results of a transmissive pulse wave sensor at a base of a finger using a surface light source of the present invention.

FIG. 10 is a chart showing an example of measurement results of a transmissive pulse wave sensor at a base of a finger using a point light source.

FIG. 11 is a chart showing an example of measurement results of a reflective pulse wave sensor on a wrist using a surface light source of the present invention.

FIG. 12 is a chart showing an example of measurement results of a reflective pulse wave sensor on a wrist using a point light source.

FIG. 13 is a schematic cross-sectional view showing an example of a configuration of a wavelength conversion film provided with a red light cut filter according to the present invention.

### EMBODIMENTS TO CARRY OUT THE INVENTION

The light emitting member of the present invention is a light emitting member that emits near-infrared light, and includes a surface light source that emits at least red light and a wavelength conversion film that converts visible light of the surface light source into near-infrared light. It has a maximum emission wavelength in the wavelength range of 700 to 1500 nm. This feature is a technical feature common to or corresponding to each of the following embodiments.

As an embodiment of the present invention, from the viewpoint of exhibiting the effect of the present invention, it is preferable that the wavelength conversion film contains a luminescent dye, and that the luminescent dye is a squarylium compound, a croconium compound, or a thiadiazole compound. These compounds are luminescent dyes that absorb light in the red light region (600 to 650 nm) and emit light in the near-infrared light region (700 to 1500 nm), and do not overlap with the light absorption region of water in the living body. By overlapping with the absorption range of hemoglobin, it is effective as an excellent biometric authentication (finger vein recognition), a wristband type electronic device and a biometric device (for example, a pulse oximeter and a pulse wave sensor).

Further, when the squarylium compound is a compound having a structure represented by Formula (1), it is preferable in that the durability in the infrared light emitting region is improved, and the light emitting member has more excellent light emitting intensity and light emitting life.

Further, when the squarylium compound represented by Formula (1) is a compound having a structure represented Formula (2), it has a hydroxy group in the structure. This embodiment is preferable from the viewpoint of improving luminescent property, expanding π-conjugation in the molecule, and lengthening the maximum emission wavelength.

Further, the fact that the wavelength conversion film contains two or more kinds of luminescent dyes and further contains at least two kinds of squarylium compounds expands the range of options for adjusting the luminescent characteristics in the near-infrared light region. This embodiment is preferable in that it is possible to obtain a light emitting member having more excellent light emitting intensity and light emitting life.

Further, it is preferable to apply an organic electroluminescent element as a surface light source in that high brightness uniformity is obtained and a thin light emitting member having excellent flexibility and durability is obtained.

Further, it is preferable that the maximum emission wavelength obtained from the light emitting member is 780 nm or more in that more excellent biometric authentication performance is exhibited.

Hereinafter, the present invention and the constitution elements thereof, as well as configurations and embodiments to carry out the present invention, will be detailed in the following. In the present description, when two figures are used to indicate a range of value before and after “to”, these figures are included in the range as a lowest limit value and an upper limit value. In the description of each figure, the numbers described at the end of the components represent the reference numerals described in the drawings to be described. In addition, the dimensional ratios in the drawings are exaggerated for convenience of explanation and they may differ from the actual ratios.

### <<Light Emitting Member>>

The light emitting member of the present invention is a light emitting member that emits near-infrared light, and includes a surface light source that emits at least red light and a wavelength conversion film that converts visible light of the surface light source into near-infrared light. It is characterized by having a maximum emission wavelength in the wavelength range of 700 to 1500 nm.

### [Basic Configuration of Light Emitting Member]

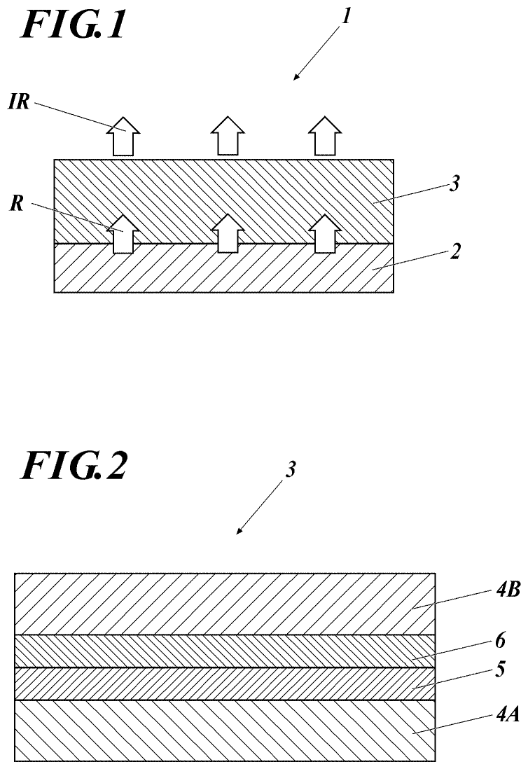

FIG. 1 shows the basic configuration of the light emitting member of the present invention.

The light emitting member 1 shown in FIG. 1 has a configuration provided with a wavelength conversion film 3 that converts visible light of the surface light source 2 into near-infrared light IR on a surface light source 2 that emits red light R, for example, an organic EL element that emits red light R.

The details of the surface light source and the wavelength conversion film will be described below.

### [Surface Light Source]

The surface light source according to the present invention is a light source having at least a characteristic of emitting red light, preferably a surface light source having a brightness uniformity of 70% or more when uniformly emitting light. As a specific surface light source, an organic electroluminescent element is preferable.

The surface light source referred to in the present invention is a light source that emits uniform light from the entire wide surface, and as a light source for this, there is a point light source typified by a light emitting diode (light emitting diode: LED). Compared to the point light source, the surface light source generally has no uneven emission brightness and does not cast a shadow. Therefore, in addition to lighting of living room space or backlighting such as a display, it may be suitably used for lighting of an imaging device for biometric authentication. When used in an imaging device for biometric authentication, the surface light source preferably includes an organic electroluminescent element (hereinafter, also referred to as “organic EL element” or “OLED”), or μLED (micro LED).

In the surface light source according to the present invention, in order to utilize the flexibility of the OLED, it is more preferable to use a flexible resin film as the base material. Examples thereof are: polyesters such as polyethylene terephthalate (PET) and polyethylene naphthalate (PEN), polyethylene, polypropylene, cellophane, cellulose esters and their derivatives such as cellulose diacetate, cellulose triacetate (TAC), cellulose acetate butyrate, cellulose acetate propionate (CAP), cellulose acetate phthalate, and cellulose nitrate, polyvinylidene chloride, polyvinyl alcohol, polyethylene vinyl alcohol, syndiotactic polystyrene, polycarbonate, norbornene resin, polymethyl pentene, polyether ketone, polyimide, polyether sulfone (PES), polyphenylene sulfide, polysulfone, polyether imide, polyether ketone imide, polyamide, fluororesin, Nylon, polymethyl methacrylate, acrylic resin, polyallylate and cycloolefin resins such as ARTON (trade name, made by JSR Co. Ltd.) and APEL (trade name, made by Mitsui Chemicals, Inc.).

### (Organic EL Element)

As the surface light source according to the present invention, it is preferable to apply an organic EL element.

An organic EL element suitable for the present invention as a surface light source includes, for example, a configuration in which an anode and a cathode are provided on a base material, and organic functional layers including a light emitting layer are sandwiched between the anode and the cathode at opposite positions may be mentioned. Further, each functional layer such as a sealing member, a gas barrier layer, and a light extraction layer may be appropriately combined and configured according to the purpose.

Representative element constitutions used for an organic EL element of the present invention are as follows, however, the element constitutions applicable to an organic EL element of the present invention is not limited to these.

(1) Anode/light emitting layer/cathode
(2) Anode/light emitting layer/electron transport layer/cathode
(3) Anode/hole transport layer/light emitting layer/cathode
(4) Anode/hole transport layer/light emitting layer/electron transport layer/cathode
(5) Anode/hole transport layer/light emitting layer/electron transport layer/electron injection layer/cathode
(6) Anode/hole injection layer/hole transport layer/light emitting layer/electron transport layer/cathode
(7) Anode/hole injection layer/hole transport layer/(electron blocking layer/) light emitting layer/(hole blocking layer/) electron transport layer/electron injection layer/cathode.

The details of each specific constituent layer constituting the organic EL element applicable to the present invention and the manufacturing method thereof are not particularly limited, and known constituent materials and manufacturing methods may be applied. For example, the contents described in JP-A 2013-089608, JP-A 2014-120334, JP-A 2015-201508, and WO 2018/51617 may be referred to.

### (Brightness Uniformity)

In the surface light source according to the present invention, it is preferable that the brightness uniformity defined by the following equation (1) is 70% or more when the surface light source is uniformly emitted.

When, for example, an LED is applied as a point light source, the brightness uniformity is about 50% or less. When it is applied as a light source for biometric authentication without processing, the non-uniform brightness portion (unevenness) of the light source is erroneously recognized as an image of the object to be measured, which may lead to a decrease in the authentication rate in many cases.

In the present invention, from the viewpoint of guaranteeing the authentication accuracy as the biometric authentication device, the brightness uniformity is preferably about 80%, more preferably 90% or more.

The brightness uniformity of the surface light source may be determined by using a spectral radiance meter (CS-1000) manufactured by Konica Minolta, Inc., for example, as described in JP-A 2007-265850 and JP-A 2009-21336 by measuring the radiance distribution of the surface light source.

Specifically, the surface light source (for example, an organic EL element) is driven and emitted under the condition that the emission brightness is 1000 cd/cm², and the light emitting surface is divided into minute regions, for example, 2 to 5 mm square, and the brightness of each region is divided. The minimum brightness (cd/cm²) and the maximum brightness (cd/cm²) are obtained, respectively, and the brightness uniformity is evaluated by the following equation (1).

Brightness uniformity (%)=[Minimum brightness on the light emitting surface (cd/cm²)/Maximum brightness on the light emitting surface (cd/cm²)]×100 Equation (1)

In the present invention, as a method for further improving the brightness uniformity of the surface light source, the following methods may be mentioned, for example: increasing the feeding points of the electrodes, forming the electrodes in a grid shape, arranging the anode and the cathode to minimize the distance between them, and preparing the surface light source by a coating method instead of vapor deposition. It is also preferable to appropriately select each of these techniques or combine a plurality of these methods.

### [Wavelength Conversion Film]

As shown in FIG. 1, the light emitting member of the present invention is characterized by including a wavelength conversion film that converts visible light of the surface light source into near-infrared light in addition to the surface light source described above.

Furthermore, the wavelength conversion film according to the present invention preferably contains a luminescent dye having a wavelength conversion ability. As long as the wavelength conversion film according to the present invention contains a luminescent dye having a wavelength conversion ability, the form and production method are not particularly limited, and the wavelength conversion film is appropriately determined according to the intended use.

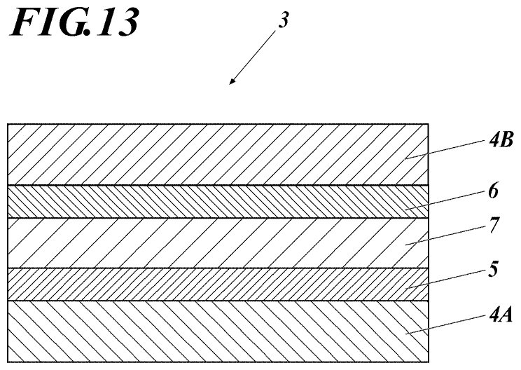

As a form, it may be manufactured separately from the surface light source and used by superimposing it on the surface light source, or may be laminated on the surface light source. Alternatively, it may also serve as an adhesive or a sealing film. Further, as shown in FIG. 13, a red light cut filter for excluding red light emitted without wavelength conversion may be laminated or included. The thickness is also appropriately determined depending on the application, but is preferably in the range of 0.01 to 500 μm from the viewpoint of flexibility and miniaturization. More preferably, it is in the range of 0.1 to 200 μm, and still more preferably in the range of 1 to 100 μm.

Examples of the method for producing the wavelength conversion film include a method in which a composition for forming a wavelength conversion film containing a luminescent dye may be temporarily or permanently applied onto the support. Examples thereof are a the vapor deposition method, a sputtering method, a spin coating method, a gravure coating method, and a dip coating method. Further, in the case of manufacturing by a wet method such as a spin coating method, the solvent used is not particularly limited. For example, water, alcohol-based, diol-based, ketone-based, ester-based, ester-based, aromatic hydrocarbon-based (may contain halogen), and aliphatic or aliphatic hydrocarbon-based solvents may be mentioned.

Further, a known resin material may be used as the matrix material in order to dissolve or disperse the luminescent dye when preparing the composition for forming a wavelength conversion film. A non-polar resin material is preferably used from the viewpoint of not affecting the emission wavelength of the luminescent dye. Examples thereof are: polyolefins such as polystyrene, polyethylene, polypropylene and polymethylpentene, acrylic resins such as polymethylmethacrylate, ethylene-vinyl acetate copolymer (abbreviation: EVA), polyvinyl butyrate (abbreviation: PVB), triacetyl cellulose (abbreviation: PVB). bbbreviation: TAC), and cellulose esters such as nitrocellulose.

Further, if necessary, in addition to the luminescent dye, various well-known additives such as a colorant, a light stabilizer, an antioxidant, a surfactant, a flame retardant, an inorganic additive, a clearing agent, an ultraviolet absorber, a filler, and a light scattering particle may be contained.

### (Light Scattering Particles)

Among the above, the light scattering particles are particles having a function of multiple scattering the light that has entered the wavelength conversion film. By adding these, the optical path length of the light entering the wavelength conversion film is extended, and the chance of wavelength conversion inside the wavelength conversion film is increased, so that the wavelength conversion efficiency is improved. Further, the light returned to the inside of the wavelength conversion film is scattered again by the reflection at the wavelength conversion film interface, so that the light extraction efficiency is expected to be improved.

The average particle size of the light-scattering particles is preferably 0.01 μm or more and 10 μm or less, more preferably 0.1 μm or more and 5 μm or less, and still more preferably 0.2 μm or more and 1 μm or less. When the average particle size of the light-scattering particles is less than 0.01 μm, sufficient light-scattering property cannot be obtained in the wavelength conversion film, and in order to obtain sufficient light-scattering property, it is necessary to increase the addition amount the light-scattering particles. On the other hand, when the average particle size of the light-scattering particles exceeds 10 μm, the number of light-scattering particles decreases even if the addition amount (mass %) is the same, so that the number of scattering points decreases and a sufficient light-scattering effect may not be obtained.

The shape of the light-scattering particles is not particularly limited, and for example, spherical (true spherical, substantially true spherical, elliptical spherical), polyhedral, rod-shaped (cylindrical, prismatic), flat plate, flaky, and indefinite shape may be cited. When the shape of the light-scattering particles is not spherical, the particle diameter of the light-scattering particles may be a true spherical value having the same volume.

The light-scattering particles are not particularly limited and may be appropriately selected depending on the intended purpose. They may be organic fine particles or inorganic fine particles, but the higher the refractive index, the better the scattering performance of the particles. Therefore, of these, the inorganic fine particles having a high refractive index are preferable.

Examples of the organic fine particles having a high refractive index include polymethylmethacrylate beads, acrylic-styrene copolymer beads, melamine beads, polycarbonate beads, styrene beads, crosslinked polystyrene beads, polyvinyl chloride beads and benzoguanamine-melamine formaldehyde beads.

Examples of the inorganic fine particles having a high refractive index include inorganic oxide particles composed of at least one oxide of silicon, zirconium, titanium, indium, zinc, antimony, cerium, niobium, or tungsten. Specific examples of the inorganic oxide particles include SiO₂, ZrO₂, TiO₂, BaTIO₃, In₂O₃, ZnO, Sb₂O₃, ITO, CeO₂, Nb₂O₅ and WO₃. Among them, TiO₂, BaTiO₃, ZrO₂, CeO₂ and Nb₂O₅ are preferable, and TiO₂ is the most preferable. Further, among TiO₂, the rutile type is preferable to the anatase type because the catalytic activity is low, the weather resistance of the film is high, and the refractive index is also high.

Further, in order to contain these particles in the wavelength conversion film, those subjected to surface treatment or those not subjected to surface treatment may be selected and used from the viewpoint of improving dispersibility and stability when used as a dispersion liquid.

When surface treatment is performed, specific materials for surface treatment include different inorganic oxides such as silicon oxide and zirconium oxide, metal hydroxides such as aluminum hydroxide, and organic acids such as organosiloxane and stearic acid. These surface treatment materials may be used alone, or a plurality of types may be used in combination. Among them, from the viewpoint of the stability of the dispersion liquid, the surface treatment material is preferably a deferent inorganic oxide, or a metal hydroxide, and more preferably it is a metal hydroxide.

The content of the light-scattering particles with respect to the total solid content mass of the wavelength conversion film is preferably 0.1% by mass or more and 20% by mass or less, and more preferably 0.2% by mass or more and 5% by mass or less. When the content of the light scattering particles is less than 0.1% by mass, the light scattering effect may not be sufficiently obtained. On the other hand, when the content of the light-scattering particles exceeds 20% by mass, the transmittance is lowered because there are too many light-scattering particles.

### (Red Light Cut Filter)

Examples of the material of the red light cut filter include glass and resin, and a material that excludes red light by forming a dielectric multilayer film or a film containing an absorbing dye may be used. As the red light cut filter, a commercially available product may be used, or the above film is formed on the wavelength conversion film, or after being produced independently, it may be used in combination with a wavelength conversion film.

### (Light Emitter Having Wavelength Conversion Ability)

The light emitter contained in the wavelength conversion film according to the present invention is not limited particularly as long as it has the property of absorbing light in the red light region (600 to 650 nm) and emitting light in the near-infrared light region (700 to 1500 nm). When a light emitting member containing a light emitter is used as a biometric authentication device, it is preferable to have a light emitting maximum wavelength in the wavelength range of 750 to 1000 nm, and more preferably a light emitting maximum is in the vicinity of 850 nm from the viewpoint of guaranteeing the brightness of the obtained image. This is preferable in that it does not overlap with the water absorption region in the living body but overlaps with the hemoglobin absorption region.

Examples of the light emitter applicable to the present invention include the following. Complexes such as deuterated tris(hexafluoroacetylacetonato)neodymium (III): Nd(hfa-D₃), inorganic nanoparticles containing ions such as rare earths, quantum dot nanoparticles composed of indium arsenic and lead sulfide, water-soluble silicon nanoparticles, and further, luminescent dyes such as squarylium, cyanine, phthalocyanine, croconium rhodamine, eosin, fluorescein, triphenylmethane, and porphyrin may be used. Among these light emitters, the squarylium compound, which is a luminescent dye, is a compound that exhibits sharp absorption in the near-infrared light region, and has been used as a sensitizing dye for organic solar cells, a charge generating material for electrophotographic photosensitive members, and a fluorescent probe. Further, it has high durability in the infrared emission region and excellent luminous efficiency, and may be particularly preferably used as the luminescent dye in the present invention.

Further, the wavelength conversion film according to the present invention preferably contains at least one kind of luminescent dye, and more preferably contains two or more kinds of luminescent dyes. Furthermore, it is preferable that two or more kinds of squarylium compounds are contained as the luminescent dye.

The method for synthesizing the squarylium compound according to the present invention is not particularly limited. It may be obtained by using a known reaction described in JP-A 5-155144, JP-A 5-239366, JP-A 5-339233, JP-A 2000-345059, JP-A 2002-363434, JP-A 2004-86133, and JP-A 2004-238606.

### <Compound Having a Structure Represented by Formula (1)>

The type of the squarylium compound according to the present invention is not particularly limited, but is preferably a compound having a structure represented by the following Formula (1).

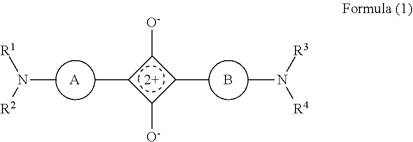

In Formula (1), A and B respectively represent an aromatic hydrocarbon ring which may have a substituent, or an aromatic heterocycle which may have a substituent, and adjacent substituents may be bonded to each other to form a ring. R¹ to R⁴ respectively represent a substituent, and at least one of R¹ to R⁴ has an aromatic hydrocarbon ring. Further, R¹ to R⁴ may be respectively bonded to each other to form a ring.

The aromatic hydrocarbon ring (it may be called as “an aromatic hydrocarbon ring group”, “an aromatic carbon ring group”, or “an aryl group”) represented by A and B is a substituted or unsubstituted aromatic hydrocarbon ring having 6 to 18 carbon atoms. Examples thereof are a phenyl group, a naphthyl group, an anthryl group, a fluorenyl group, a phenanthryl group, and a biphenylyl group. Preferably, a phenyl group, a naphthyl group, and an anthryl group may be mentioned. Examples of the aromatic heterocyclic group represented by A and B incude a pyridyl group, a pyrimidinyl group, a furyl group, a pyrrolyl group, an imidazolyl group, a benzimidazolyl group, a pyrazolyl group, a pyrazinyl group, a triazolyl group (for example, 1,2,4-triazol-1-yl group, and 1,2,3-triazol-1-yl group), a pyrazolotriazolyl group, an oxazolyl group, a benzoxazolyl group, a thiazolyl group, an isoxazolyl group, an isothiazolyl group, a furazanyl group, a thienyl group, a quinolyl group, a benzofuryl group, a dibenzofuryl group, a benzothienyl group, a dibenzothienyl group, an indolyl group, a carbazolyl group, a carbolynyl group, a diazacarbazolyl group (indicating a ring structure in which one of the carbon atoms constituting the carboline ring of the carbolynyl group is replaced with a nitrogen atom), a quinoxalinyl group, a pyridazinyl group, a triazinyl group, a quinazolinyl group, and a phthalazinyl group.

Examples of the substituent represented by R¹ to R⁴ and the substituent that may be possessed by A and B in Formula (1) are as follows: an alkyl group (for example, a methyl group, an ethyl group, a propyl group, an isopropyl group, a tert-butyl group, a pentyl group, a hexyl group, an octyl group, a dodecyl group, a tridecyl group, a tetradecyl group, and a pentadecyl group); a cycloalkyl group (for example, a cyclopentyl group and a cyclohexyl group); an alkenyl group (for example, a vinyl group and an allyl group); an alkynyl group (for example, an ethynyl group and a propargyl group); an aromatic hydrocarbon group (for example, a phenyl group, a p-chlorophenyl group, a mesityl group, a tolyl group, a xylyl group, a naphthyl group, an anthryl group, an azulenyl group, an acenaphthenyl group, a fluorenyl group, a phenantolyl group, an indenyl group, a pyrenyl group, and a biphenylyl group); an aromatic heterocyclic group (for example, a pyridyl group, a pyrazyl group, a pyrimidinyl group, a triazyl a group, a furyl group, a pyrrolyl group, an imidazolyl group, a benzimidazolyl group, a pyrazolyl group, a pyrazinyl group, a triazolyl group (for example, 1,2,4-triazol-1-yl group, and 1,2,3-triazol-1-yl group), an oxazolyl group, a benzoxazolyl group, a thiazolyl group, an isoxazolyl group, an isothiazolyl group, a furazanyl group, a thienyl group, a quinolyl group, a benzofuryl group, a dibenzofuryl group, a benzothienyl group, a dibenzothienyl group, an indolyl group, a carbazolyl group, an diazacarbazolyl group (indicating a ring structure in which one of the carbon atoms constituting the carboline ring of the carbazolyl group is replaced with a nitrogen atom), a quinoxalinyl group, a pyridazinyl group, a triazinyl group, a quinazolinyl group, and a phthalazinyl group); a heterocyclic group (for example, a pyrrolidyl group, an imidazolidyl group, a morpholyl group, and an oxazolidyl group); an alkoxy group (for example, a methoxy group, an ethoxy group, a propyloxy group, a pentyloxy group, an hexyloxy group, an octyloxy group, and a dodecyloxy group); a cycloalkoxy group (for example, a cyclopentyloxy group and a cyclohexyloxy group); an aryloxy group (for example, a phenoxy group and a naphthyloxy group); an alkylthio group (for example, a methylthio group, an ethylthio group, a propylthio group, a pentylthio group, hexylthio group, an octylthio group, and a dodecylthio group); a cycloalkylthio group (for example, a cyclopentylthio group and a cyclohexylthio group); an arylthio group (for example, a phenylthio group and a naphthylthio group); an alkoxycarbonyl group (for example, a methyloxycarbonyl group, an ethyloxycarbonyl group, a butyloxycarbonyl group, an octyloxycarbonyl group, and a dodecyloxycarbonyl group); an aryloxycarbonyl group (for example, a phenyloxycarbonyl group and a naphthyloxycarbonyl group); a sulfamoyl group (for example, an aminosulfonyl group, a methylaminosulfonyl group, a dimethylaminosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a naphthylaminosulfonyl group, and a 2-pyridylaminosulfonyl group); an acyl group (for example, an acetyl group, an ethyl carbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group, and a pyridylcarbonyl group); an acyloxy group (for example, an acetyloxy group, an ethylcarbonyloxy group, a butylcarbonyloxy group, an octylcarbonyloxy group, a dodecylcarbonyloxy group, and a phenylcarbonyloxy group); an amido group (for example, a methylcarbonylamino group, an ethylcarbonylamino group, a dimethylcarbonylamino group, a propylcarbonylamino group, a pentylcarbonylamino group, a cyclohexylcarbonylamino group, a 2-ethyhexylcarbonylamino group, an octylcarbonylamino group, a dodecylcarbonylamino group, a phenylcarbonylamino group, and a naphthylcarbonylamino group); a carbamoyl group (for example, an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethymexylaminocarbonyl group, a dodecylaminocarbonyl group, a phenylaminocarbonyl group, a naphthylaminocarbonyl group, and a 2-pyridylaminocarbonyl group); a ureido group (for example, a methylureido group, an ethylureido group, a pentylureido group, a cyclohexylureido group, an octylureido group, a dodecylureido group, a phenylureido group, a naphthylureido group, and a 2-pyridylaminoureido group); a sulfinyl group (for example, a methylsulfinyl group, an ethylsufinyl group, a butylsulfinyl group, a cyclohexylsulfinyl group, a 2-ethylhexylsulfinyl group, a dodecylsulfinyl group, a phenylsulfinyl group, a naphthylsulfinyl group, and a 2-pyridylsulfinyl group); an alkylsulfonyl group (for example, a methylsulfonyl group, an ethylsulfonyl group, a butylsulfinyl group, a cyclohexylsulfonyl group, a 2-ethylhexylsulfonyl group, and a dodecylsulfonyl group); an arylsulfonyl group or a heteroarylsulfonyl group (for example, a phenylsulfonyl group, a naphthylsulfonyl group, and a 2-pyridylsulfonyl group); an amino group (for example, an amino group, an ethylamino group, a dimethylamino group, a butylamino group, a cyclopentylamino group, a dodecylamino group, an anilino group, a naphthylamino group, and a 2-pyridylamino group); a halogen atom (for example, a fluorine atom, a chlorine atom and a bromine atom); a fluorinated hydrocarbon group (for example, a fluoromethyl group, trifluoromethyl group, a pentafluoroethyl group and a pentafluorophenyl group); a cyano group; a nitro group; a hydroxy group; a mercapto group; a silyl group (for example, a trimethylsilyl group, a triisopropylsilyl group, a triphenylsilyl group, and a phenyldiethylsilyl group) and a phosphono group. Preferred examples include an alkyl group, an aromatic hydrocarbon group, an amino group, a hydroxy group and a silyl group.

Further, these substituents may be further substituted by the above-mentioned substituents.

Of these, the substituent represented by R¹ to R⁴ is preferably an alkyl group or an aromatic hydrocarbon group.

As the alkyl group represented by R¹ to R⁴, a substituted or unsubstituted alkyl group having 6 to 10 carbon atoms is preferable. For example, a methyl group, an ethyl group, a propyl group, an isopropyl group, a butyl group, an isobutyl group, a pentyl group, a hexyl group, an octyl group, a dodecyl group, a cyclopentyl group, and a cyclohexyl group may be mentioned.

As the aromatic hydrocarbon group represented by R¹ to R⁴, a substituted or unsubstituted aromatic hydrocarbon group having 6 to 18 carbon atoms is preferable. Examples thereof are a phenyl group, a naphthyl group, an anthryl group, a fluorenyl group, a phenanthryl group, and a biphenylyl group. Preferably, a phenyl group and a naphthyl group may be mentioned.

The cyclic structure formed by the adjacent substituents may be an aromatic ring or an alicyclic ring, it may contain a hetero atom, and the cyclic structure may be a fused ring having two or more rings. The hetero atom referred to here is preferably one selected from the group consisting of a nitrogen atom, an oxygen atom and a sulfur atom. Examples of the formed ring structure include a benzene ring, a naphthalene ring, a pyridine ring, a pyridazine ring, a pyrimidine ring, a pyrazine ring, a pyrrole ring, an imidazole ring, a pyrazole ring, a triazole ring, an imidazoline ring, an oxazole ring, an isooxazole ring, a thiazole ring, an isothiazole ring, a cyclohexadiene ring, a cyclohexene ring, a cyclopentaene ring, a cycloheptatriene ring, a cycloheptadiene ring, a cycloheptaene ring, a carbazole ring, and a dibenzofuran ring.

### <Compound Having a Structure Represented by Formula (2)>

In Formula (1), it is preferable that A, B, or both of them have a hydroxy group from the viewpoint of improving luminescence. From the viewpoint of expanding the 71-conjugation in the molecule and lengthening the maximum emission wavelength, it is more preferable to be represented by the following Formula (2).

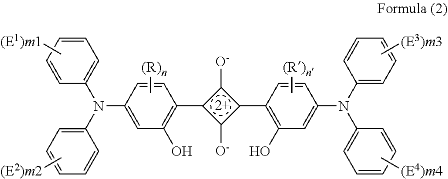

In Formula (2), E¹ to E⁴ respectively represent a substituent. m1 to m4 respectively represent 0 or an integer of 1 to 5. R and R′ respectively represent a substituent, and n and n′ respectively represent 0 or an integer of 1 to 3, provided that when m1 to m4, n and n′ are 2 or more, E¹ to E⁴, R and R′ may be the same or different.

The structure of the substituent represented by E¹ to E⁴, R and R′ is synonymous with the substituent of Formula (1) described in detail above. In particular, the substituents represented by E¹ to E⁴ are preferably alkyl groups such as a methyl group, an ethyl group and a t-butyl group, and the substituents represented by R and R′ are preferably a hydrogen atom or a hydroxy group.

Further, it is preferable that the compound having the structure represented by Formula (2) is a squarylium compound having the structure represented by the following Formula (2b).

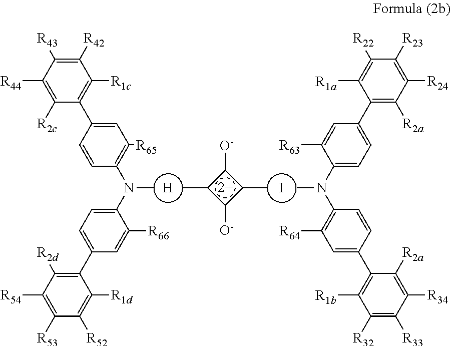

In Formula (2b), R₁ₐ to R_{1d}, R₂ₐ to R_{2d}, R₂₂ to R²⁴, R₃₂ to R₃₄, R₄₂ to R₄₄, R₅₂ to R₅₄, and R₆₃ to R₆₆ respectively represent a hydrogen atom, an alkyl group, an alkoxy group, a phenoxy group, an amino group, an aryl group or a heteroaryl group. At least one of the combinations of R₁ₐ and R₂ₐ, R_{1b} and R_{2b}, R_{1c} and R_{2c}, and R_{1d} and R_{2d} is not composed of all hydrogen atoms. R₁ₐ with R₂₂, R₂ₐ with R₂₄, R_{1b} with R₃₂, R_{2b} with R₃₄, R_{1c} with R₄₂, R₂ₑ with R₄₄, R_{1d} with R₅₂, and R_{2d} with R₅₄ may form a ring structure by bonded with each other. A ring H and a ring I each respectively represent an aryl ring that may be substituted with a hydroxy group or an alkoxy group.

In the squarylium compound having the structure represented by Formula (2b), R₁ₐ to R_{1d} and R₂ₐ to R_{2d} are preferably the same, and it is preferable that they represent an alkyl group, an alkoxy group, a phenoxy group, an amino group, an aryl group or a heteroaryl group.

In the squarylium compound having the structure represented by Formula (2b), the ring H and the ring I are the same and represent an aryl group having a hydroxy group. The above R₂₂ to R₂₄, R₃₂ to R₃₄, R₄₂ to R₄₄ and R₅₂ to R₅₄ represent the same as R₁ₐ to R_{1d} and R₂ₐ to R₂ₐ or a hydrogen atom. At least one of the combinations of R₁ₐ and R₂ₐ, R_{1b} and R_{2b}, R_{1c} and R_{2c}, R_{1d} and R_{2d} is not composed of all hydrogen atoms, and it is preferable that R₂₂, R₂₄, R₃₂, R₃₄, R₄₂, R₄₄, R₅₂ and R₅₄ represent a hydrogen atom.

Further, it is preferable that the ring H and the ring I are the same and represent an aryl group having a hydroxy group. It is preferable that R₁ₐ with R₂₂, R₂ₐ with R₂₄, R_{1b} with R₃₂, R_{2b} with R₃₄, R_{1c} with R₄₂, R_{2c} with R₄₄, R_{1d} with R₅₂, and R_{2d} with R₅₄ are respectively bonded to form a 5-membered ring or 6-membered ring structure with a carbon chain.

The squarylium compound according to the present invention may be synthesized with the method described in Chemistry of Materials, Vol. 23, p. 4789 (2011) and The Journal of Physical Chemistry, Vol. 91, p. 5184 (1987), for example. Or it may be synthesized by referring to the method described in the reference documents described in these documents.

Representative examples of the luminescent dye, the squarylium compound, the compound represented by Formula (1), and the compound represented by Formula (2) contained in the wavelength conversion film according to the present invention are shown below, but the present invention is not limited thereto.

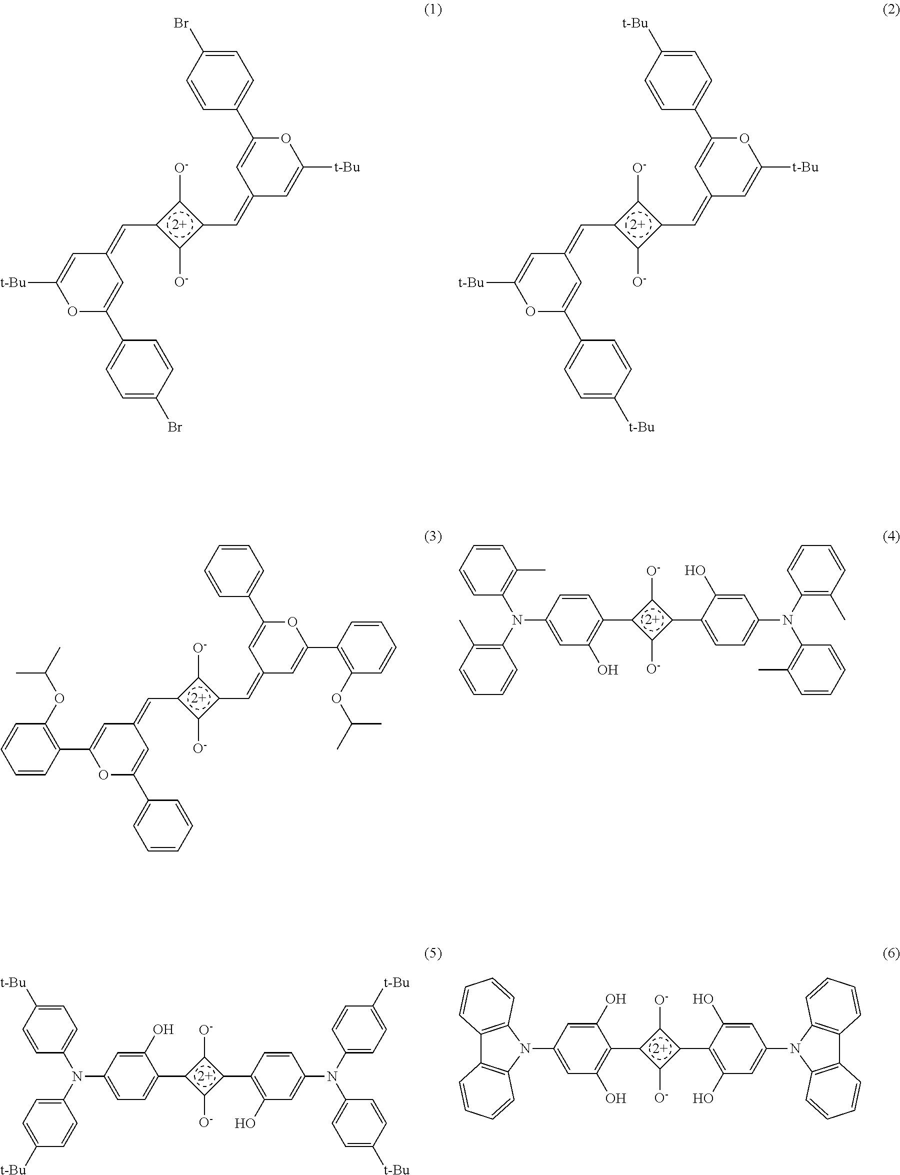

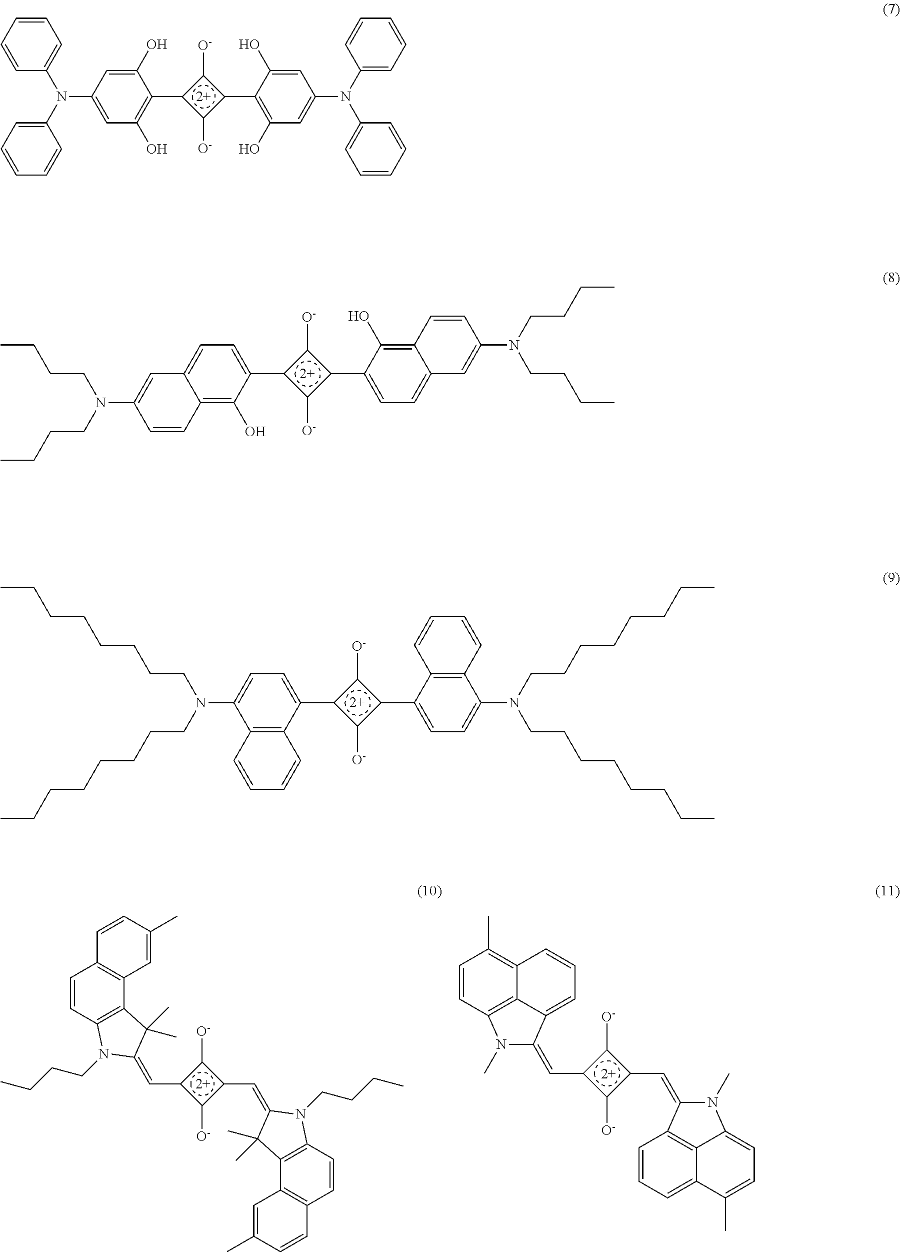

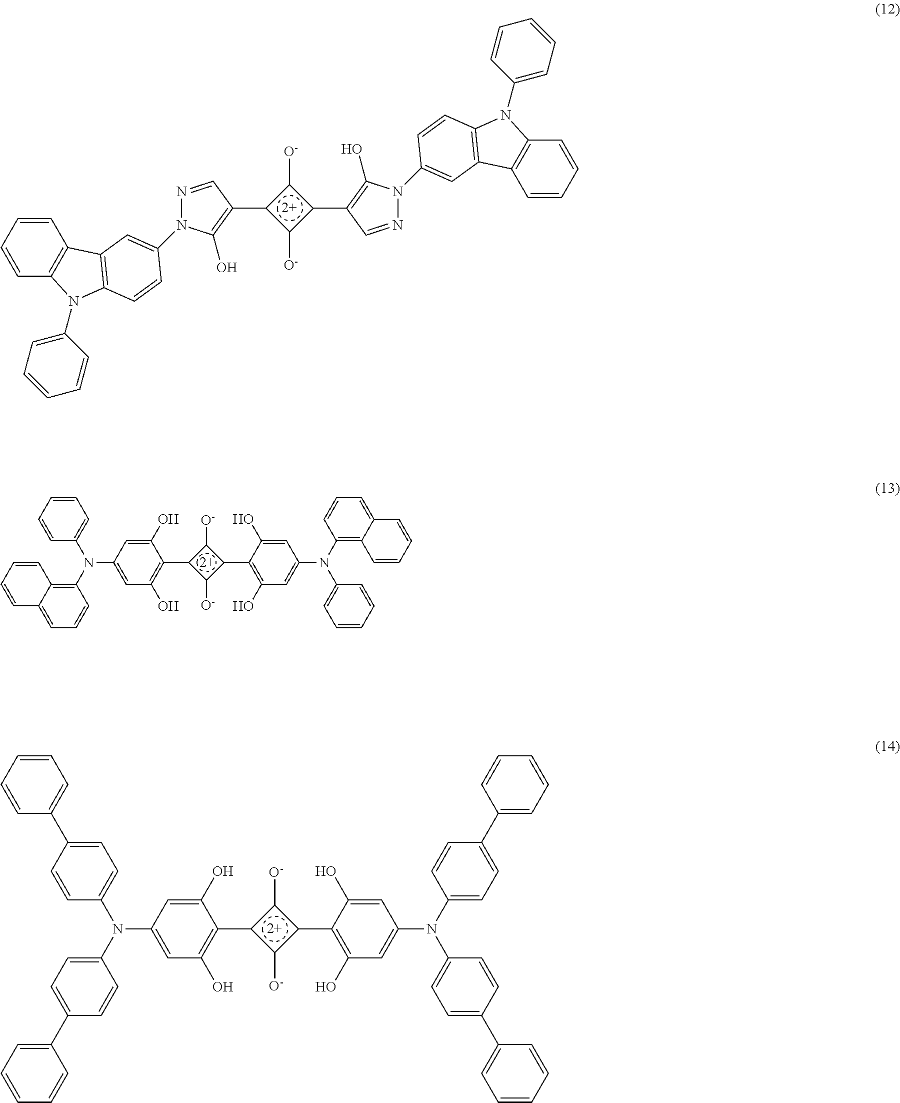

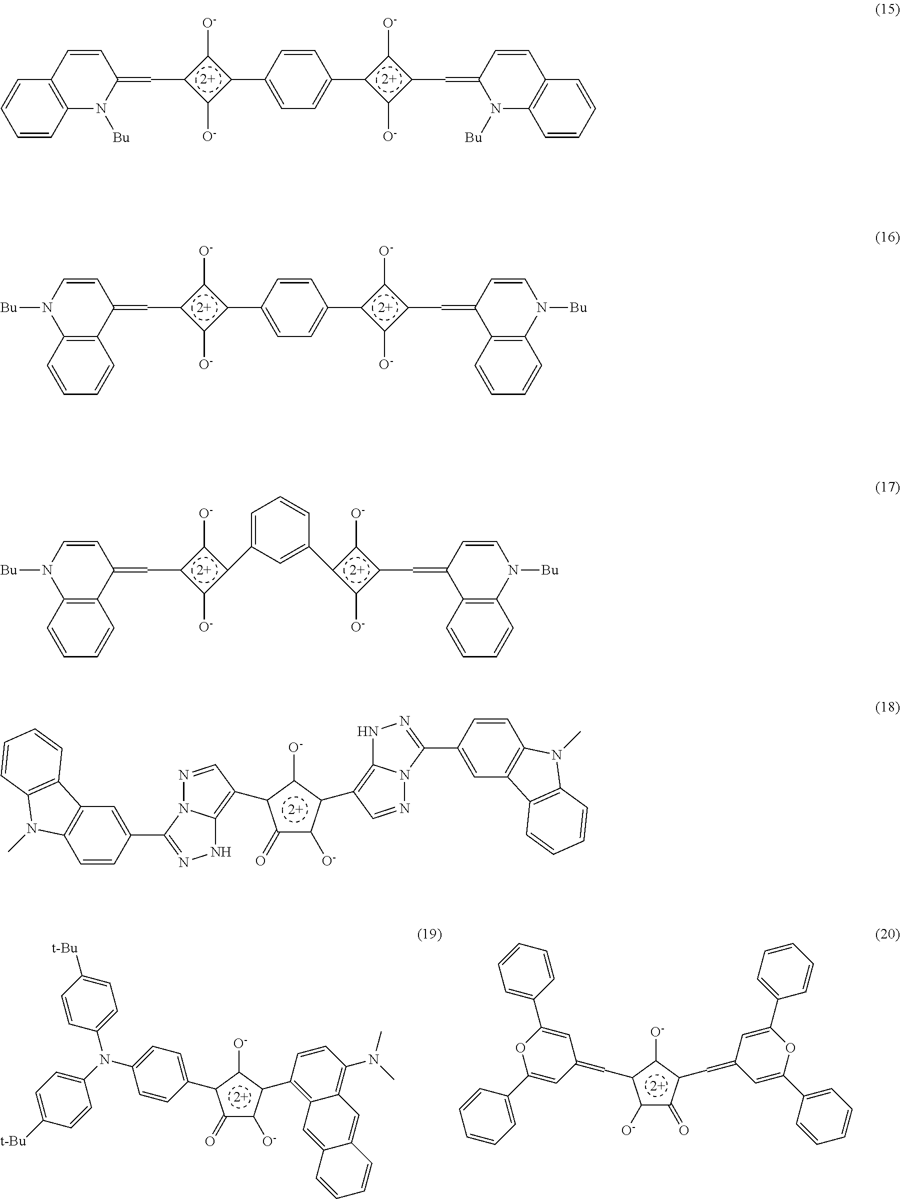

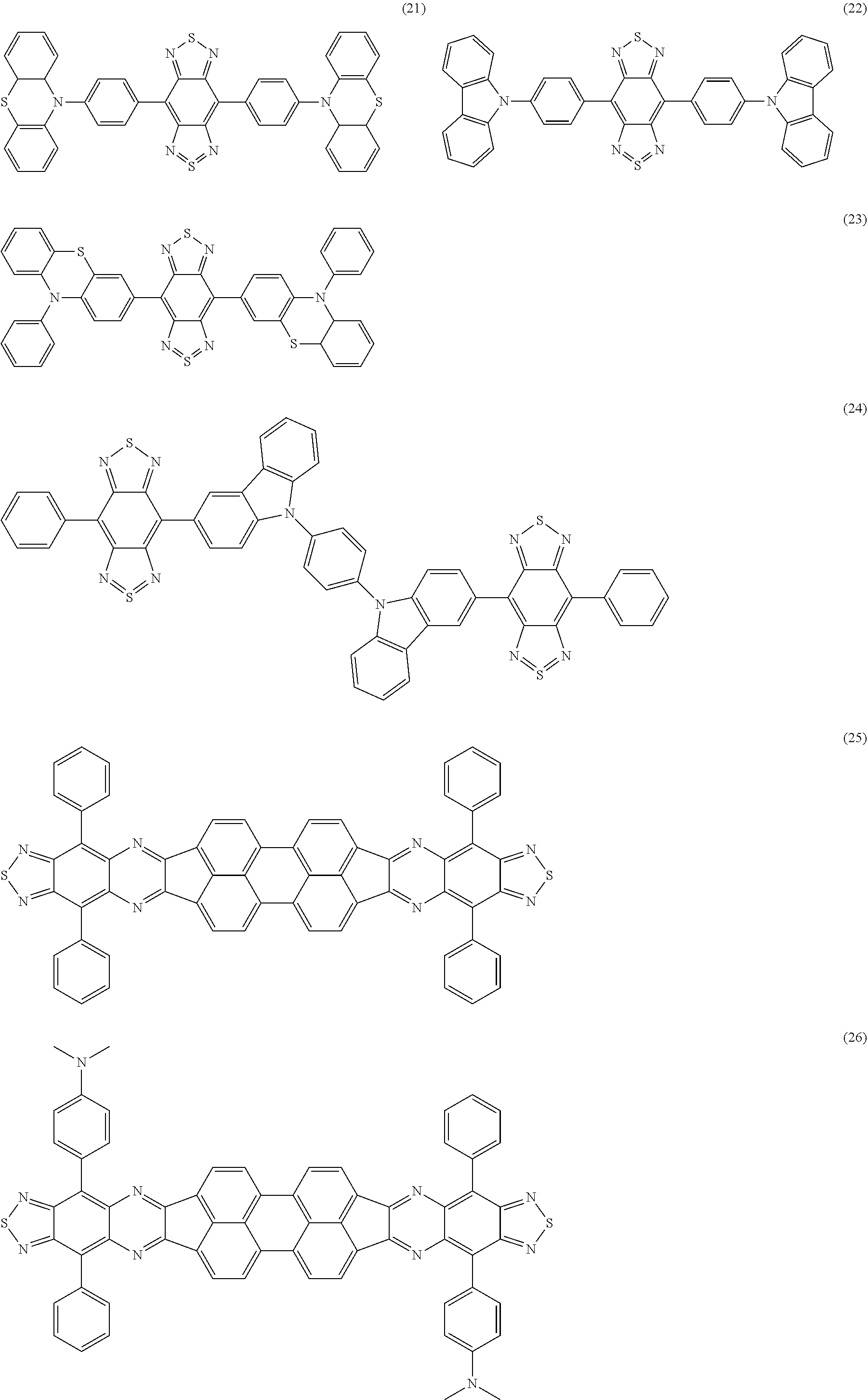

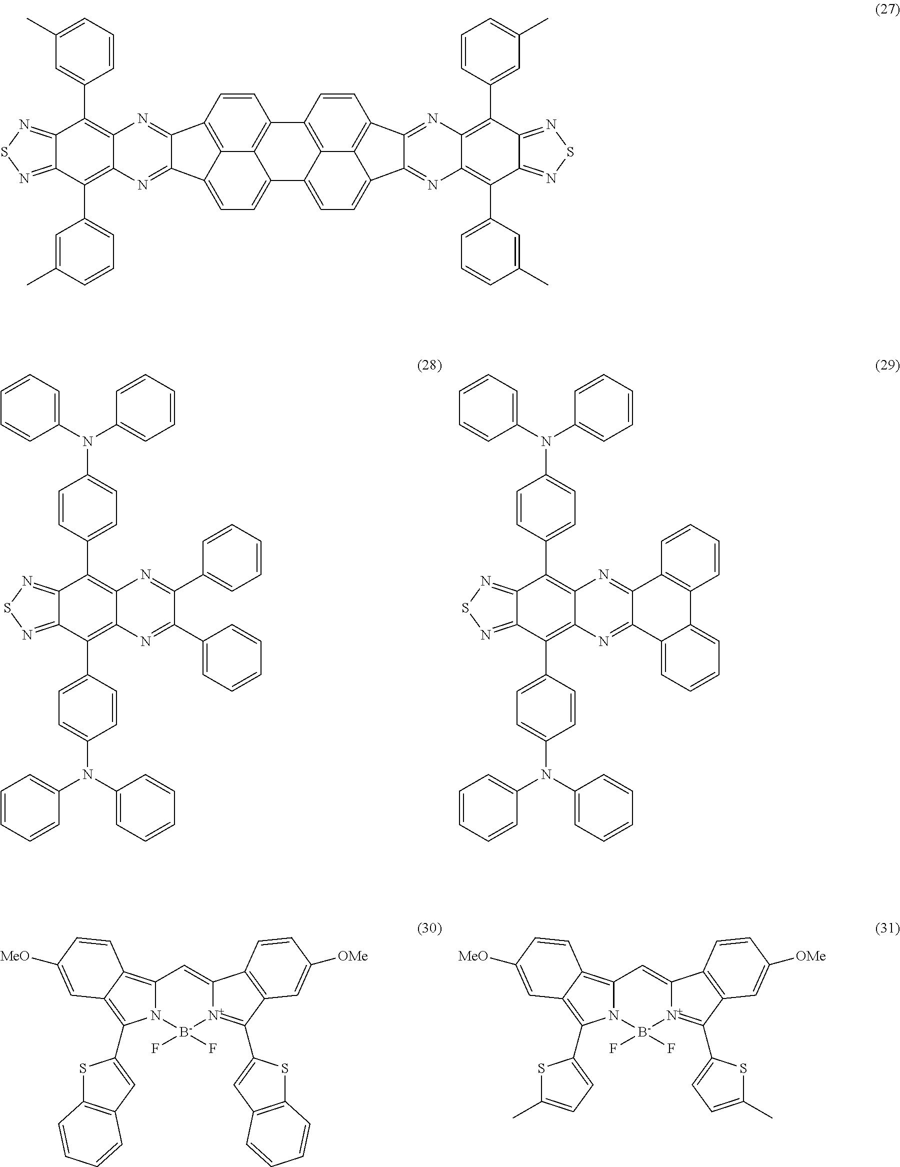

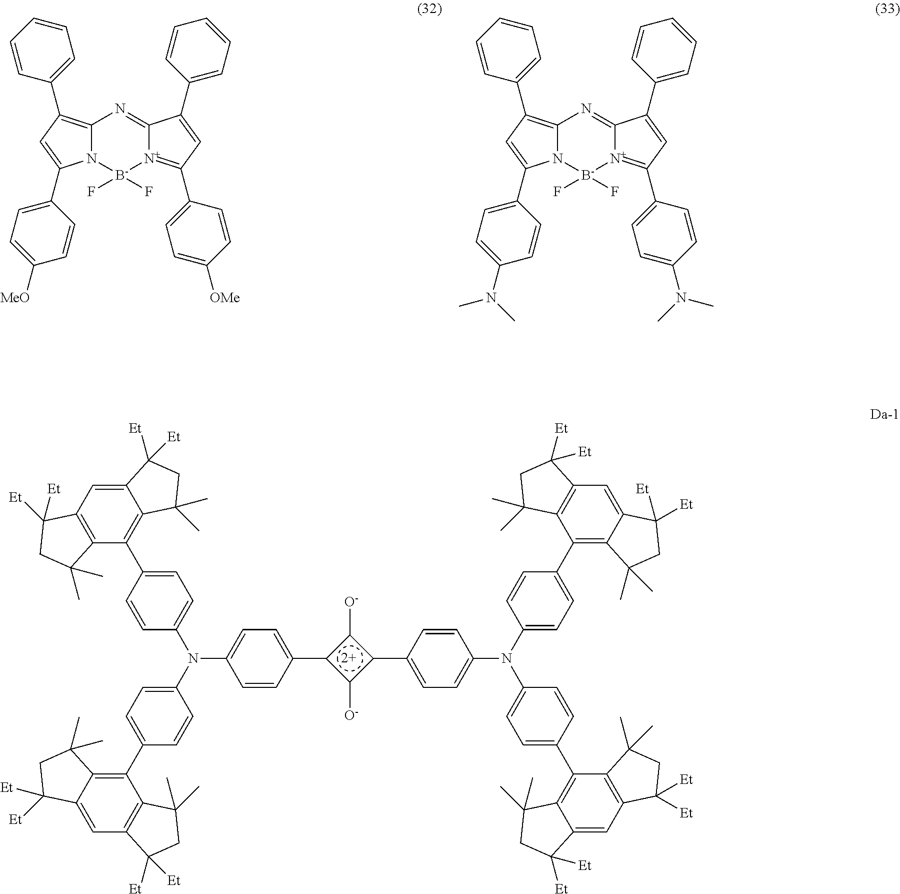

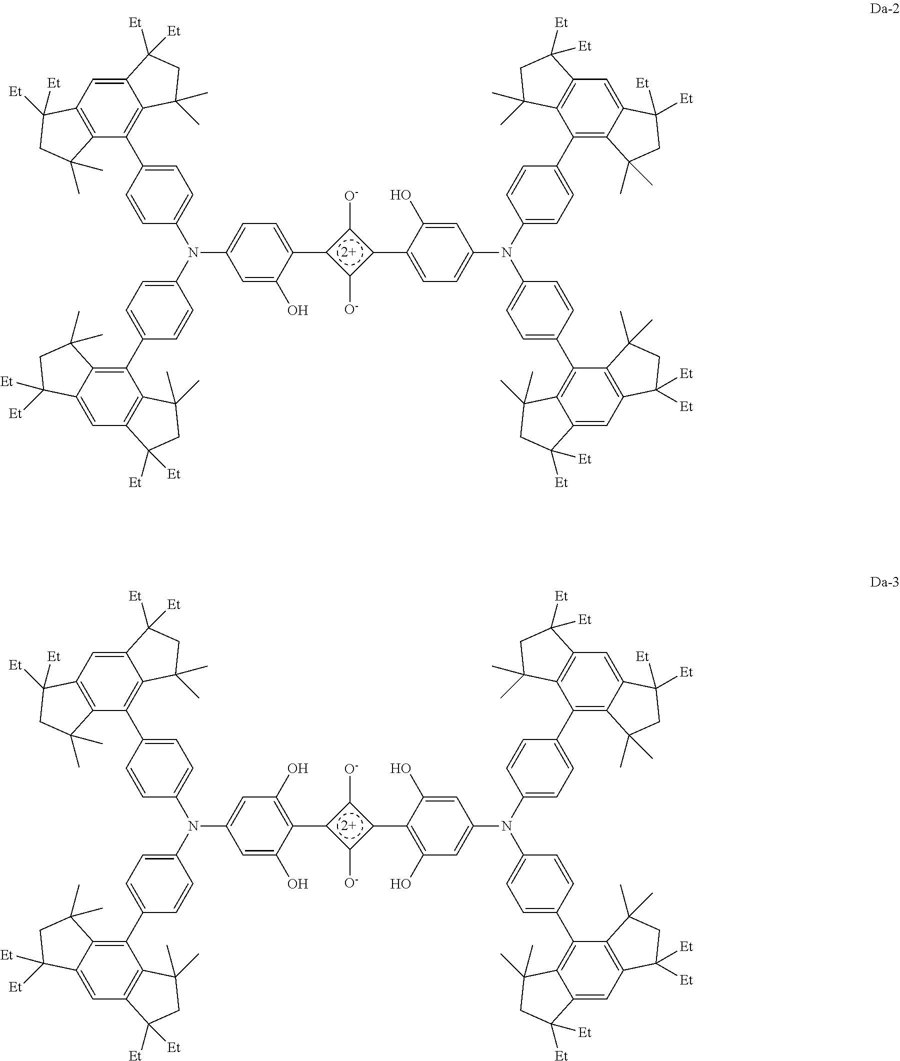

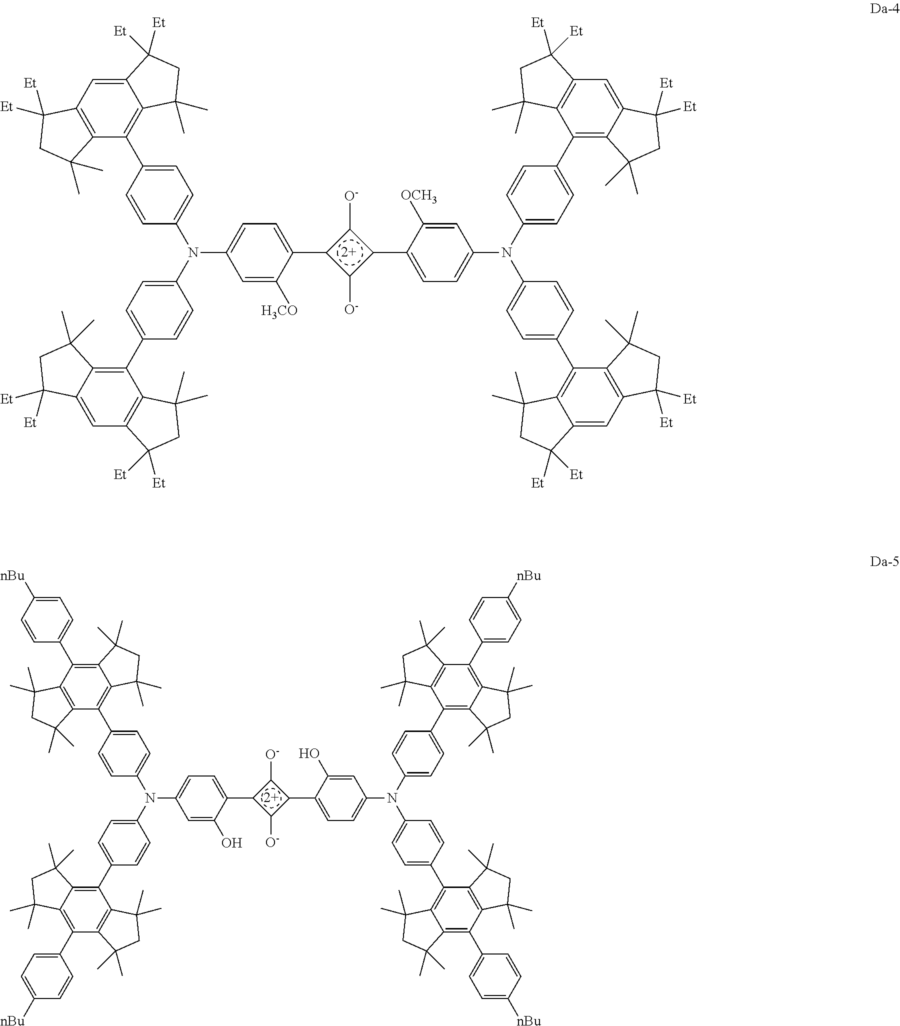

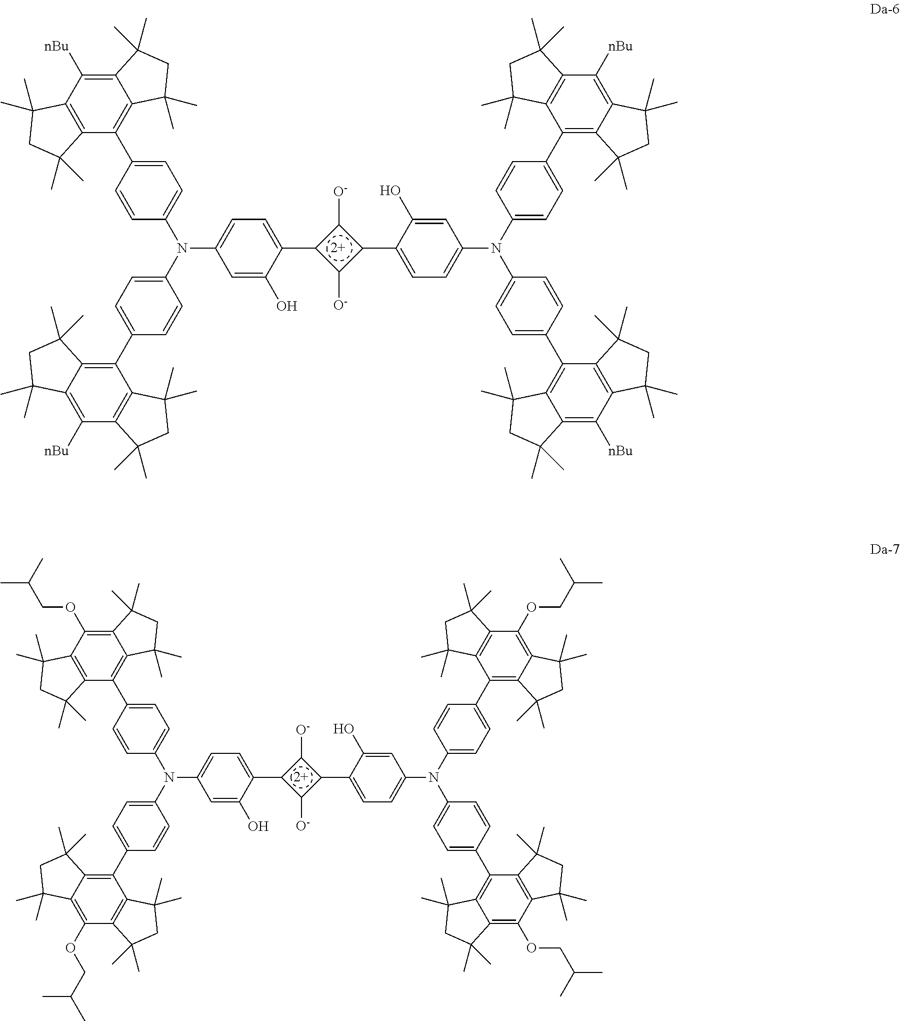

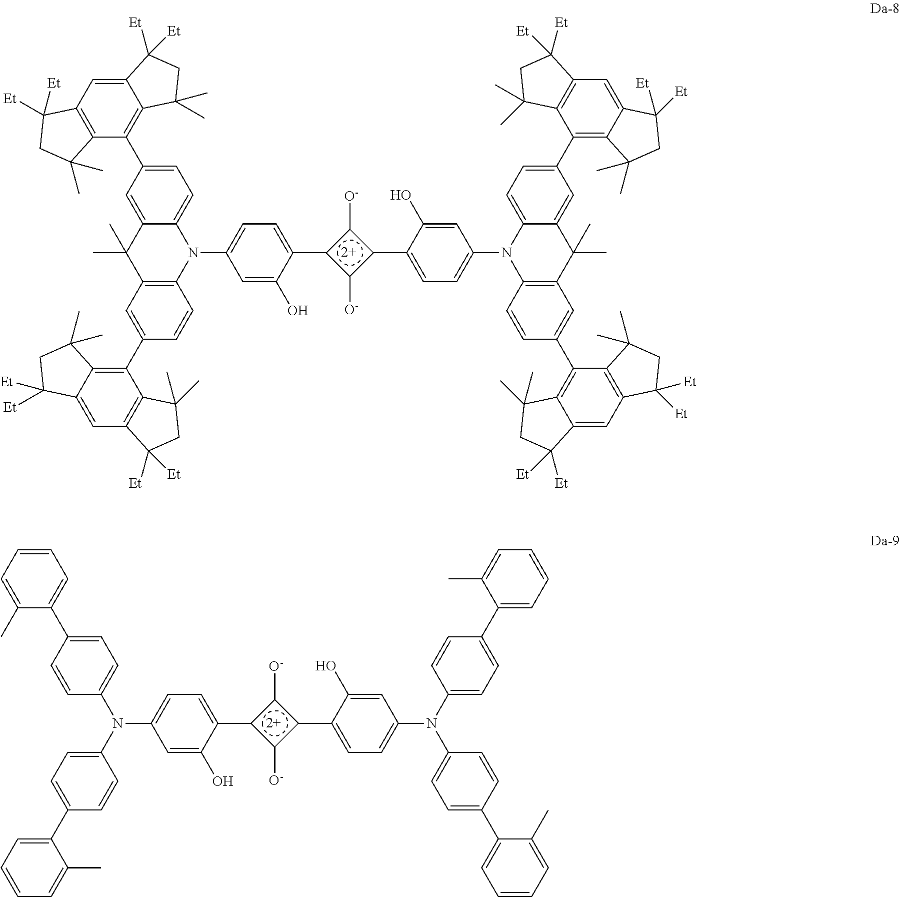

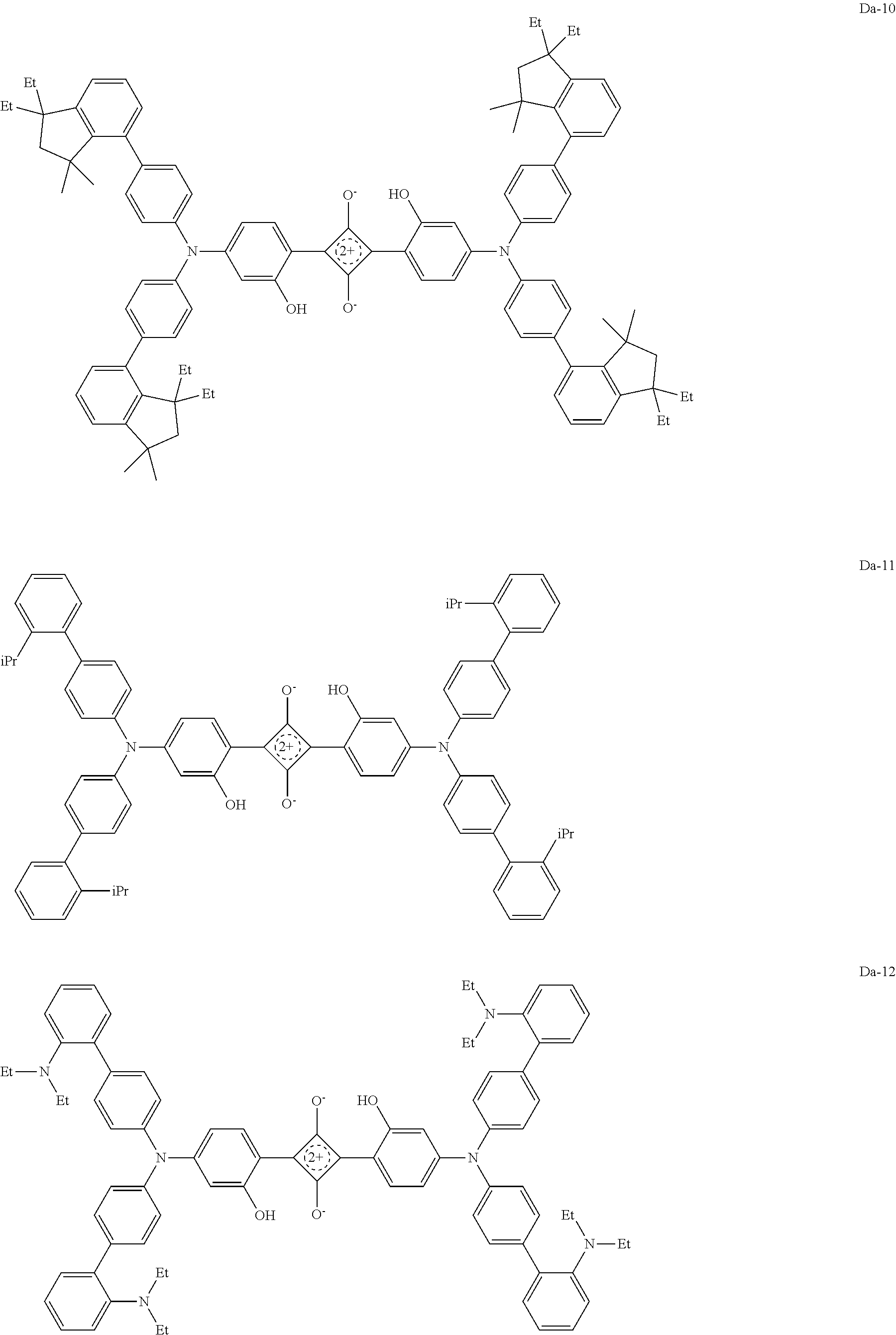

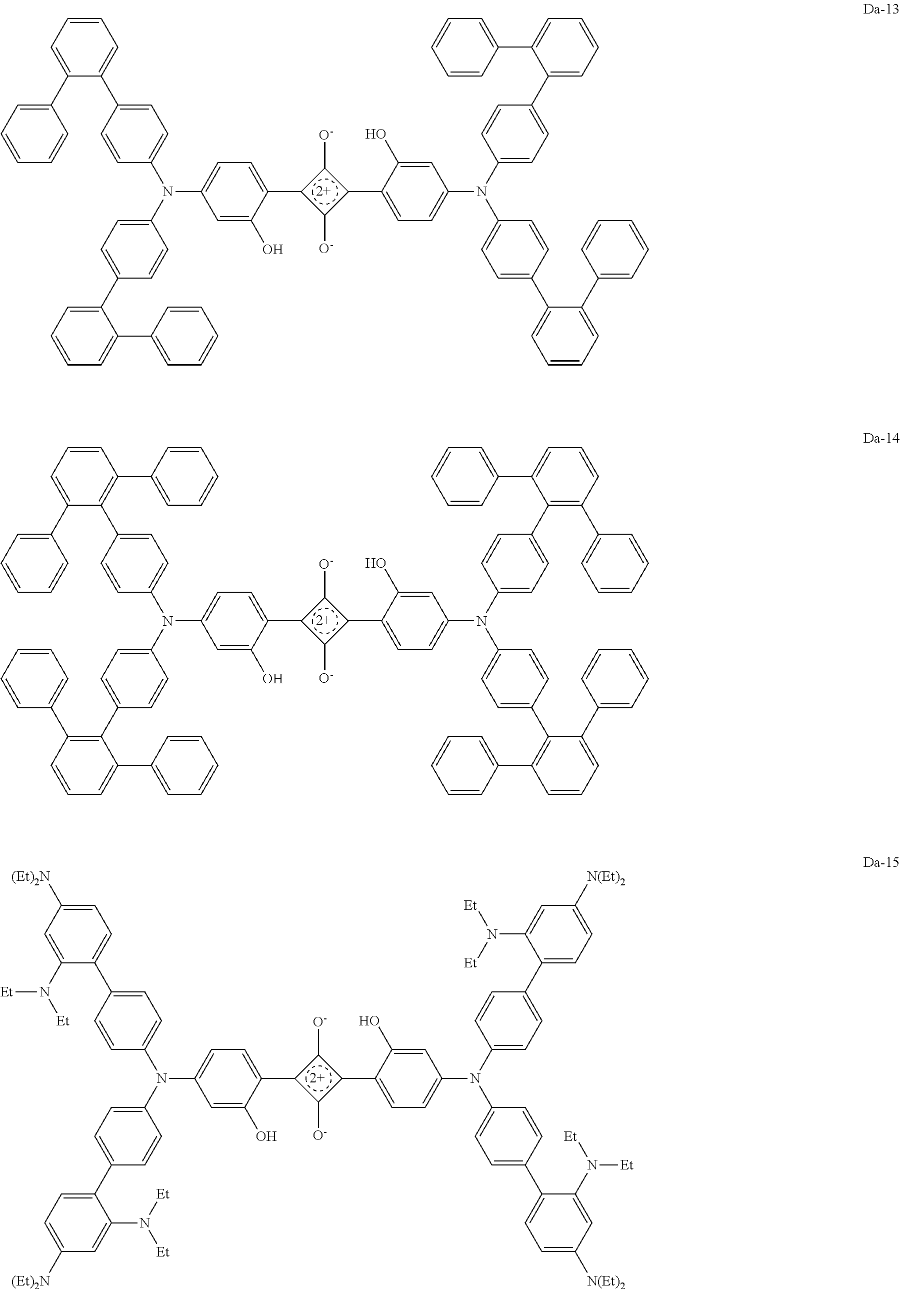

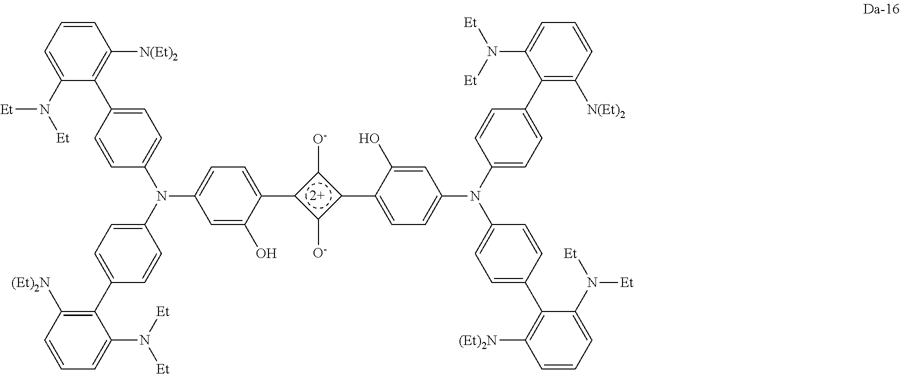

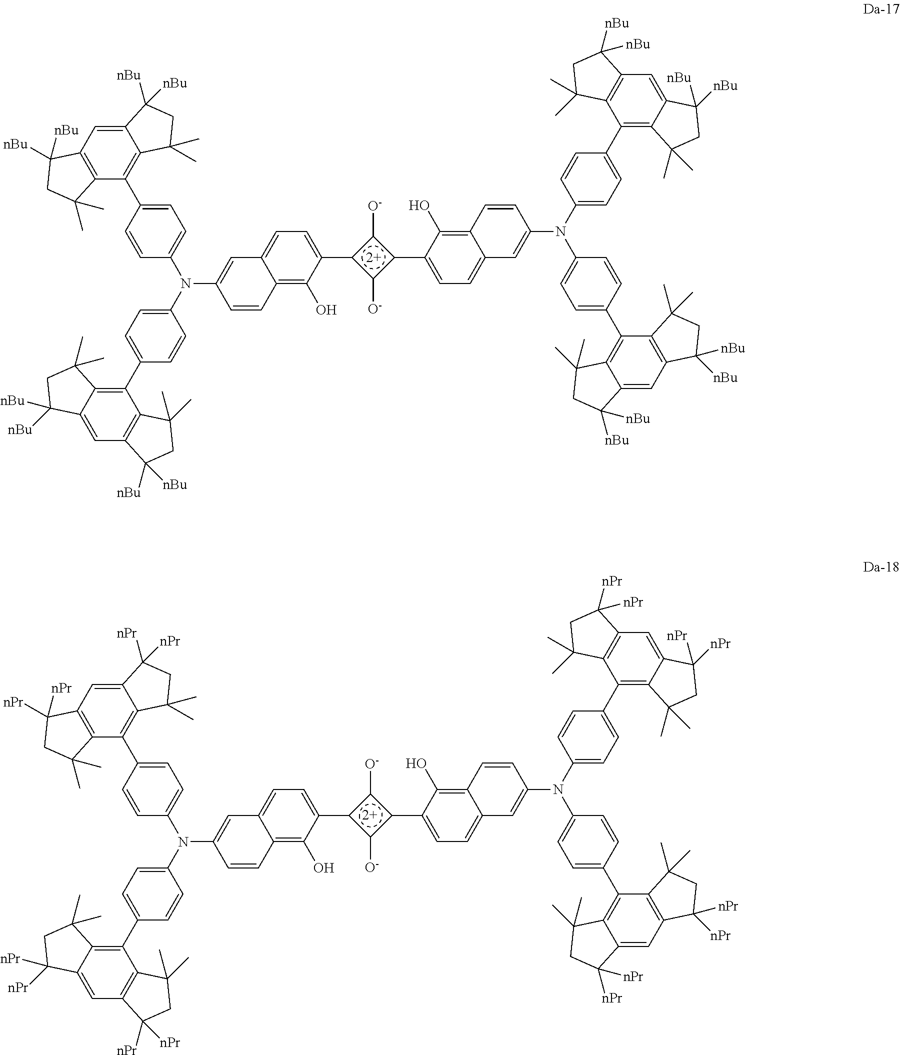

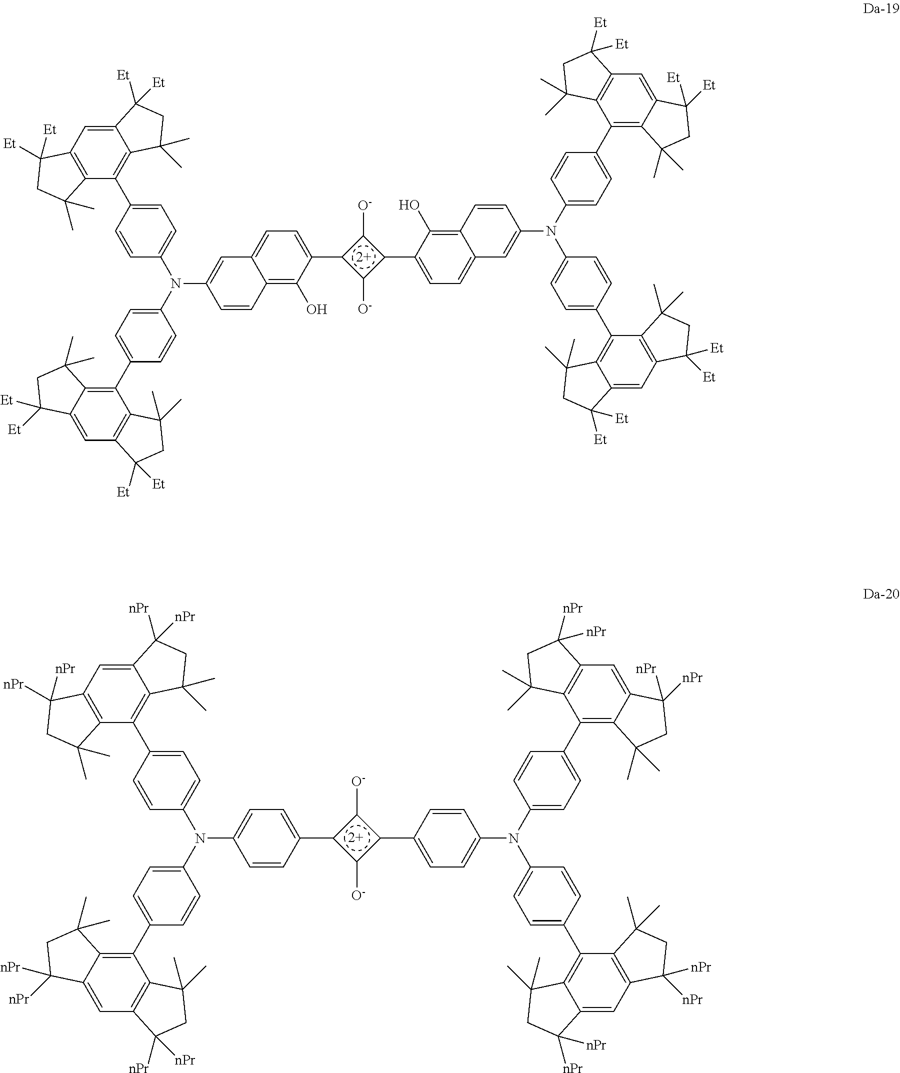

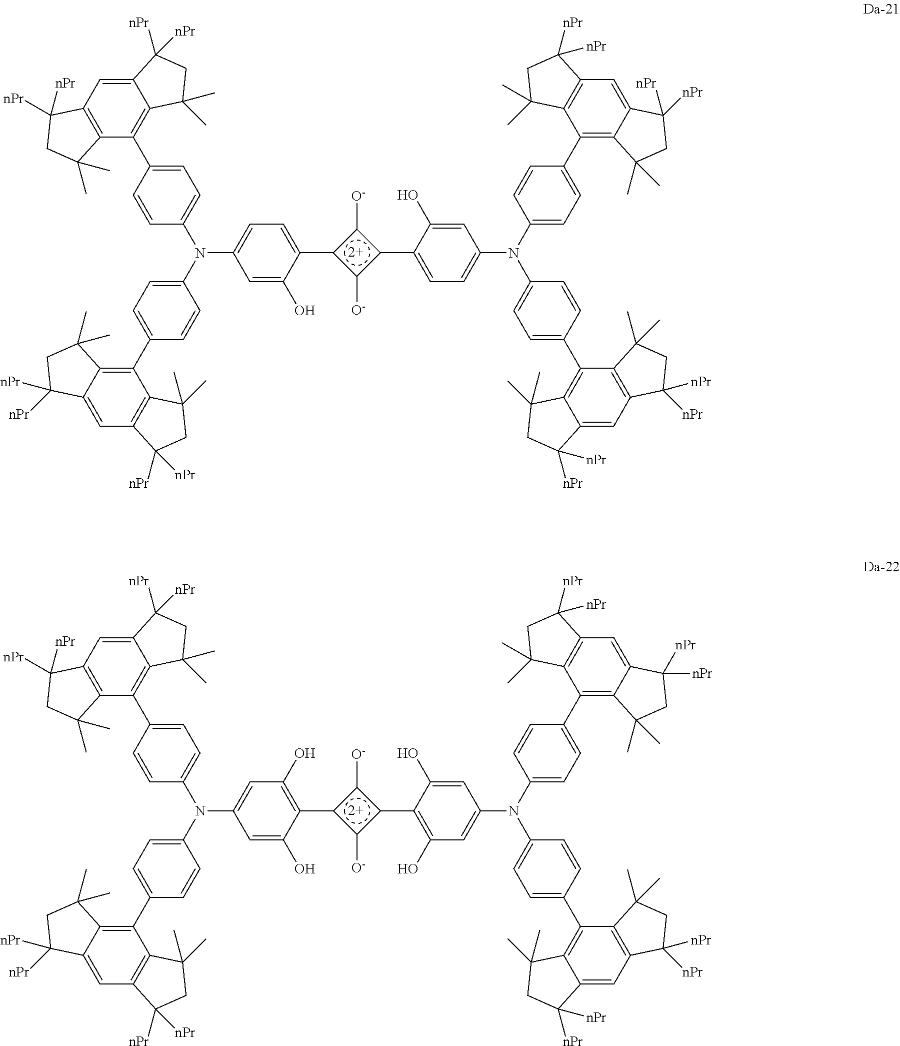

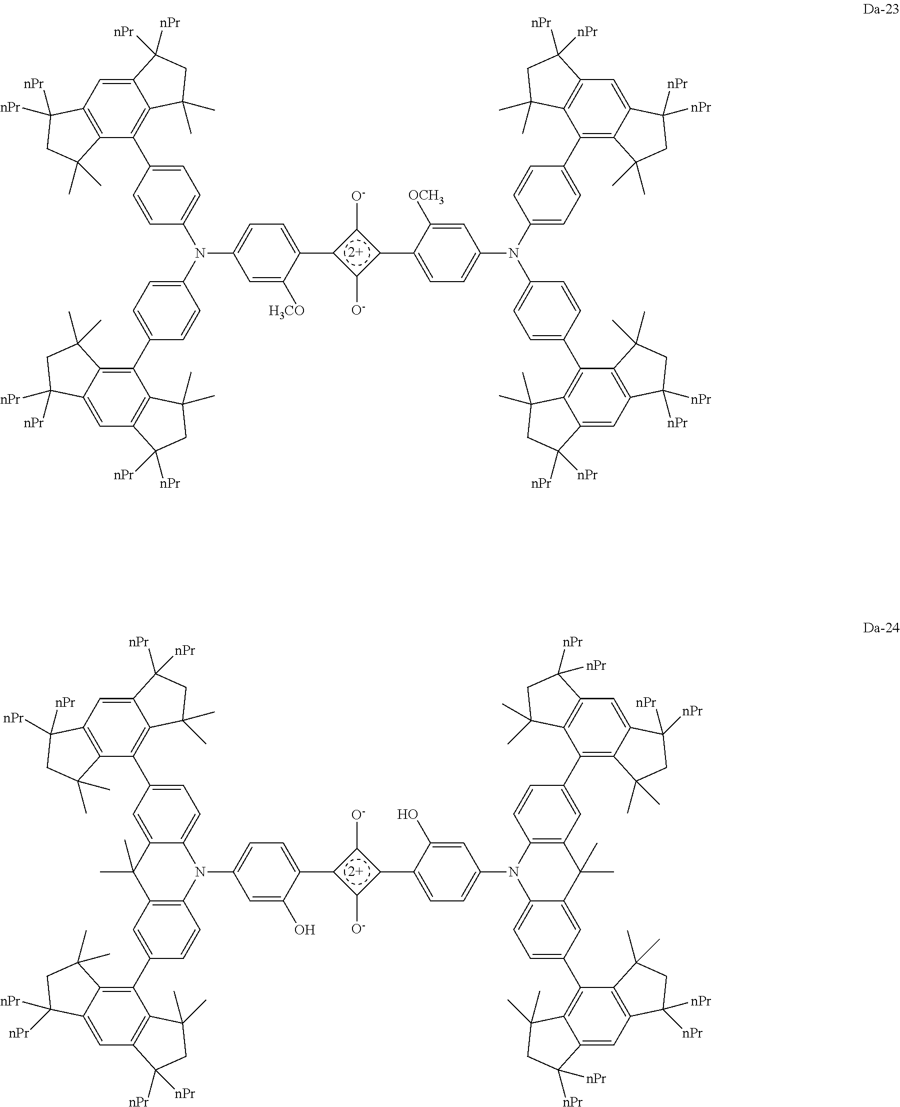

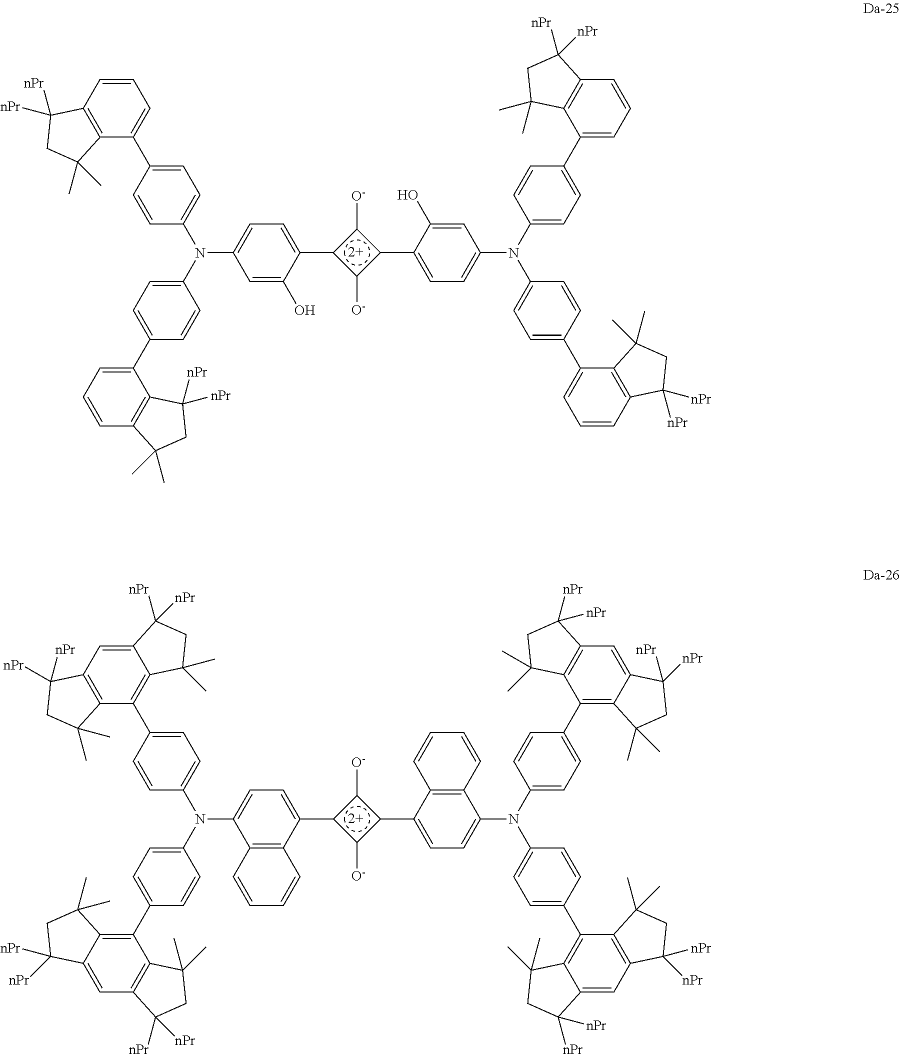

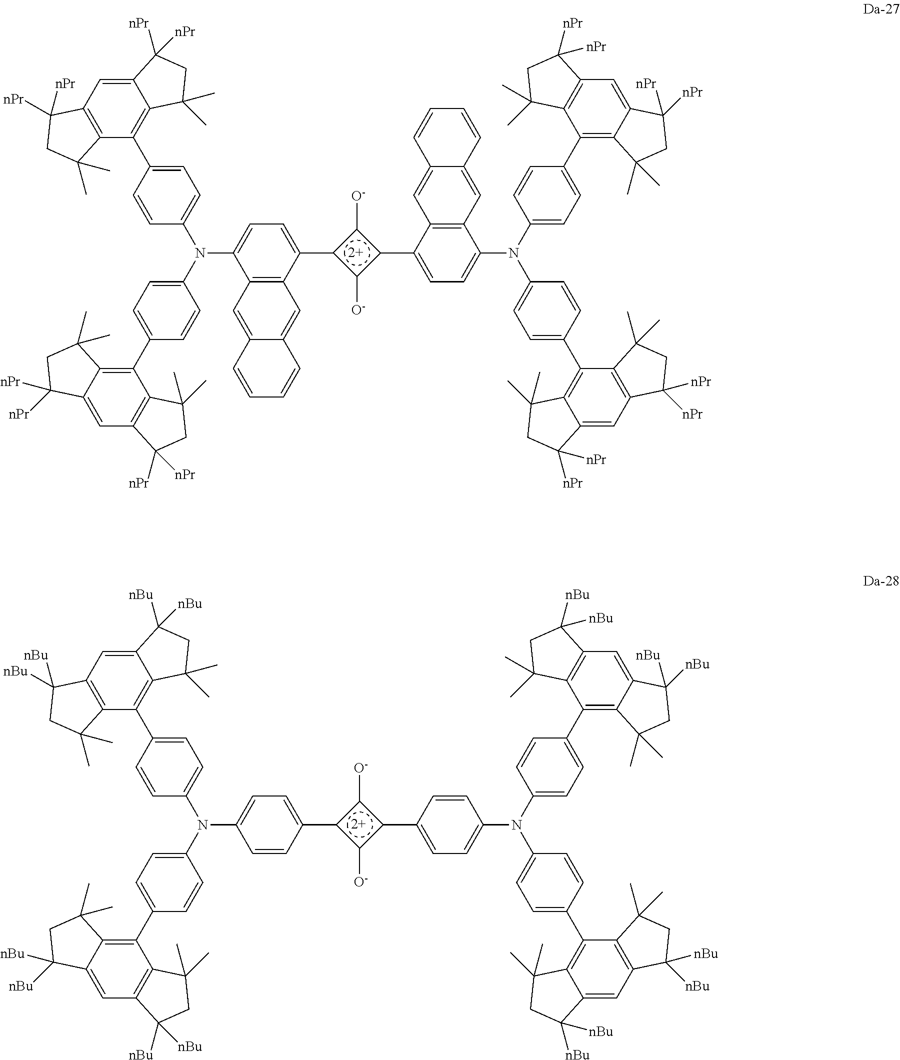

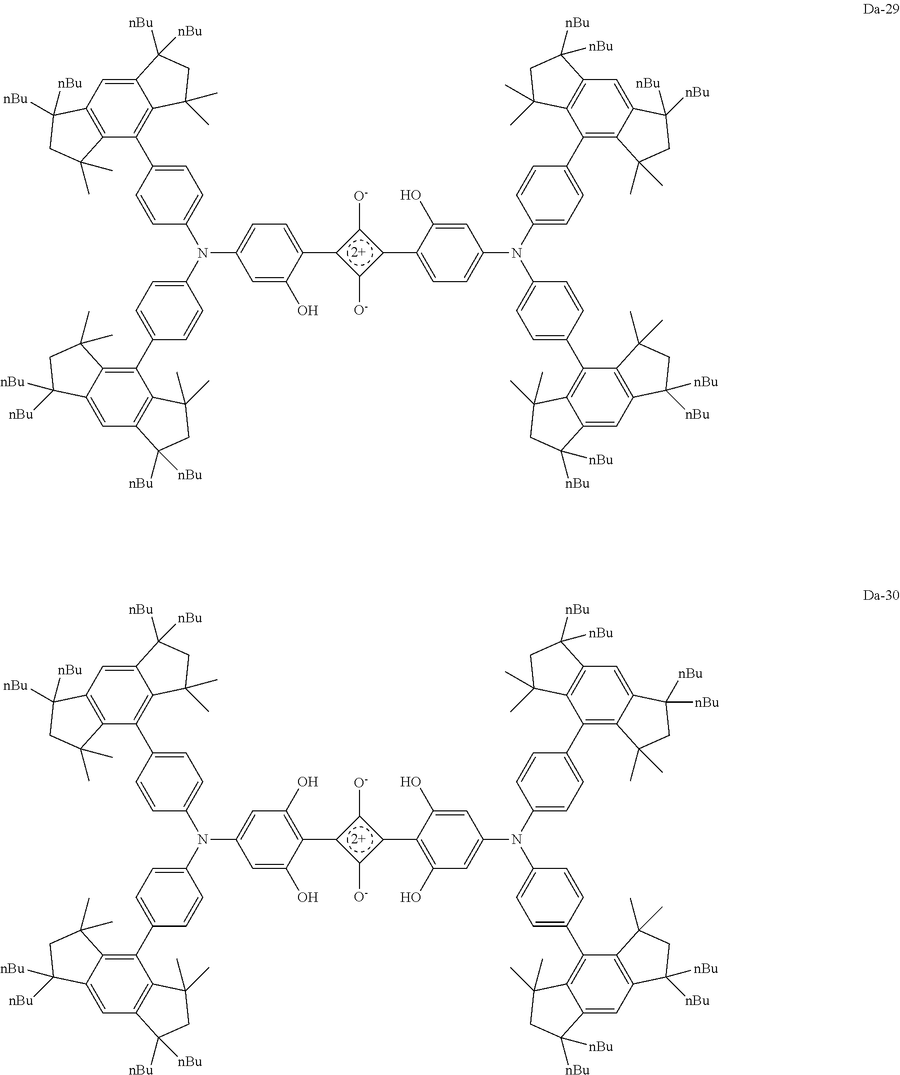

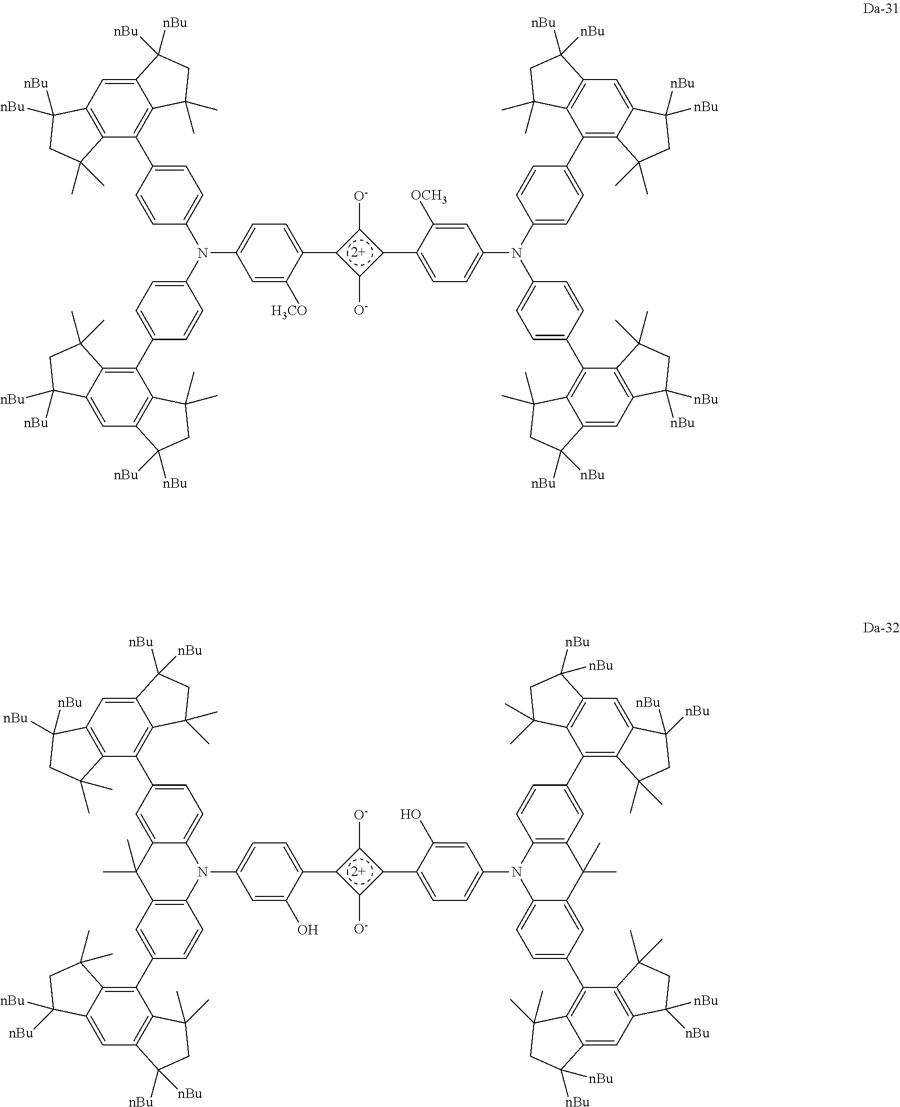

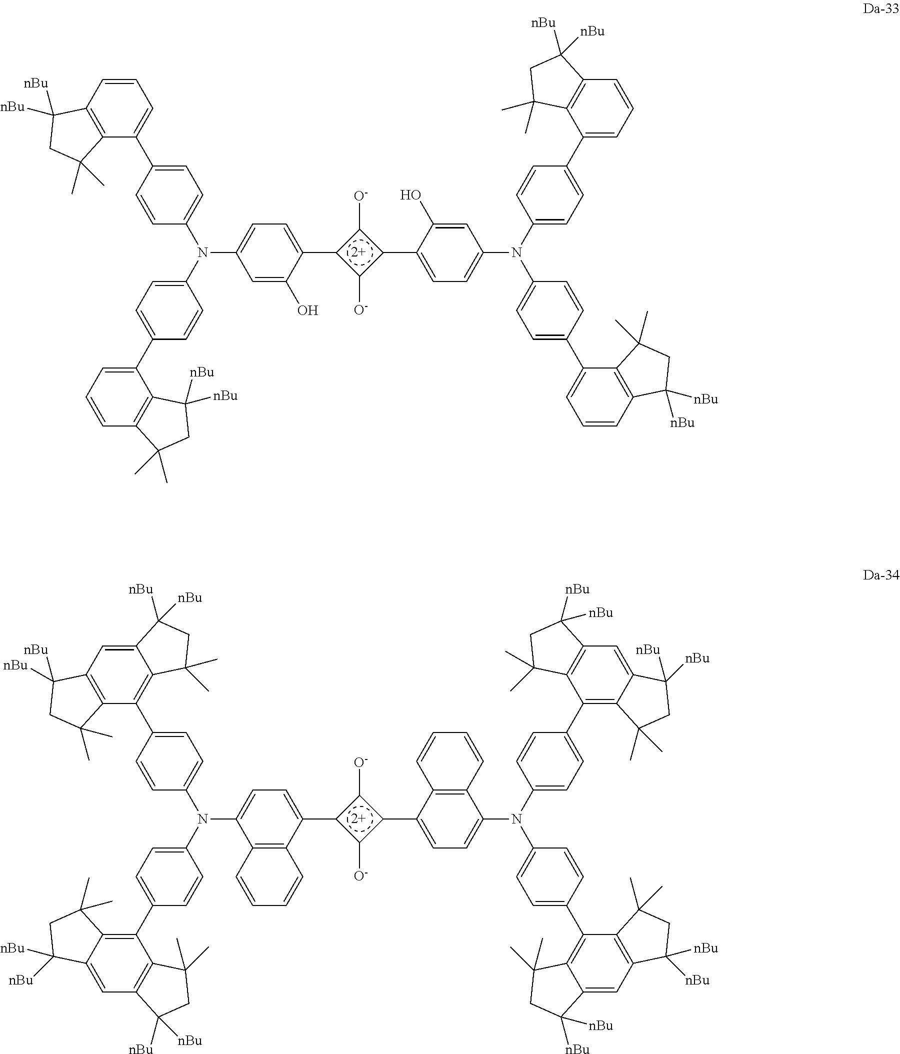

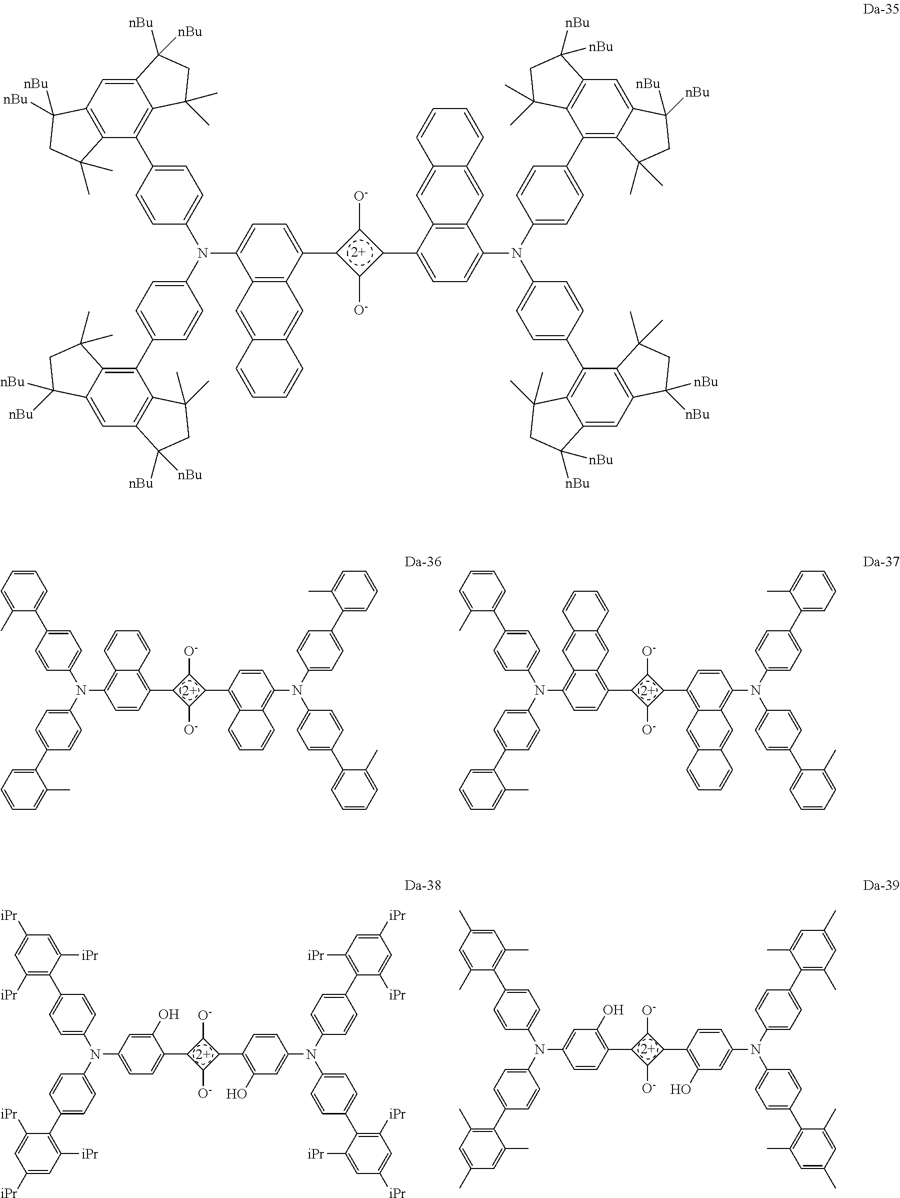

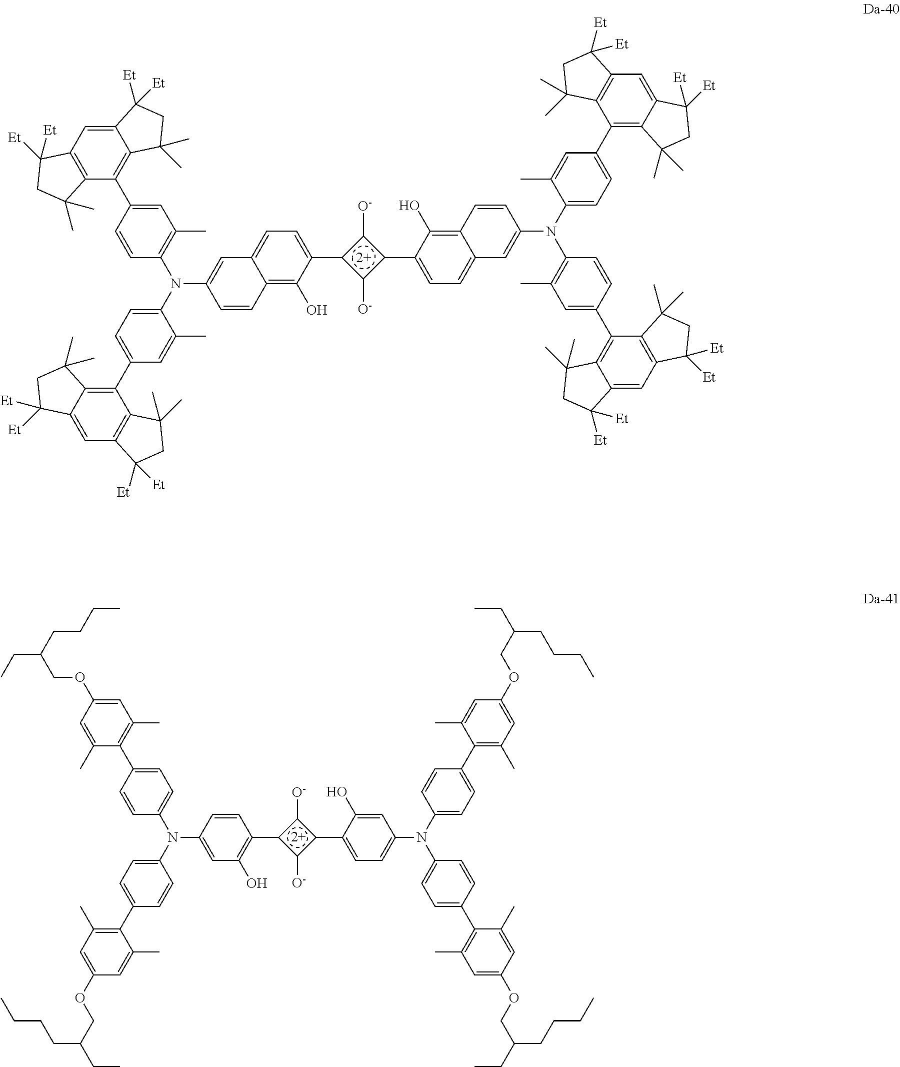

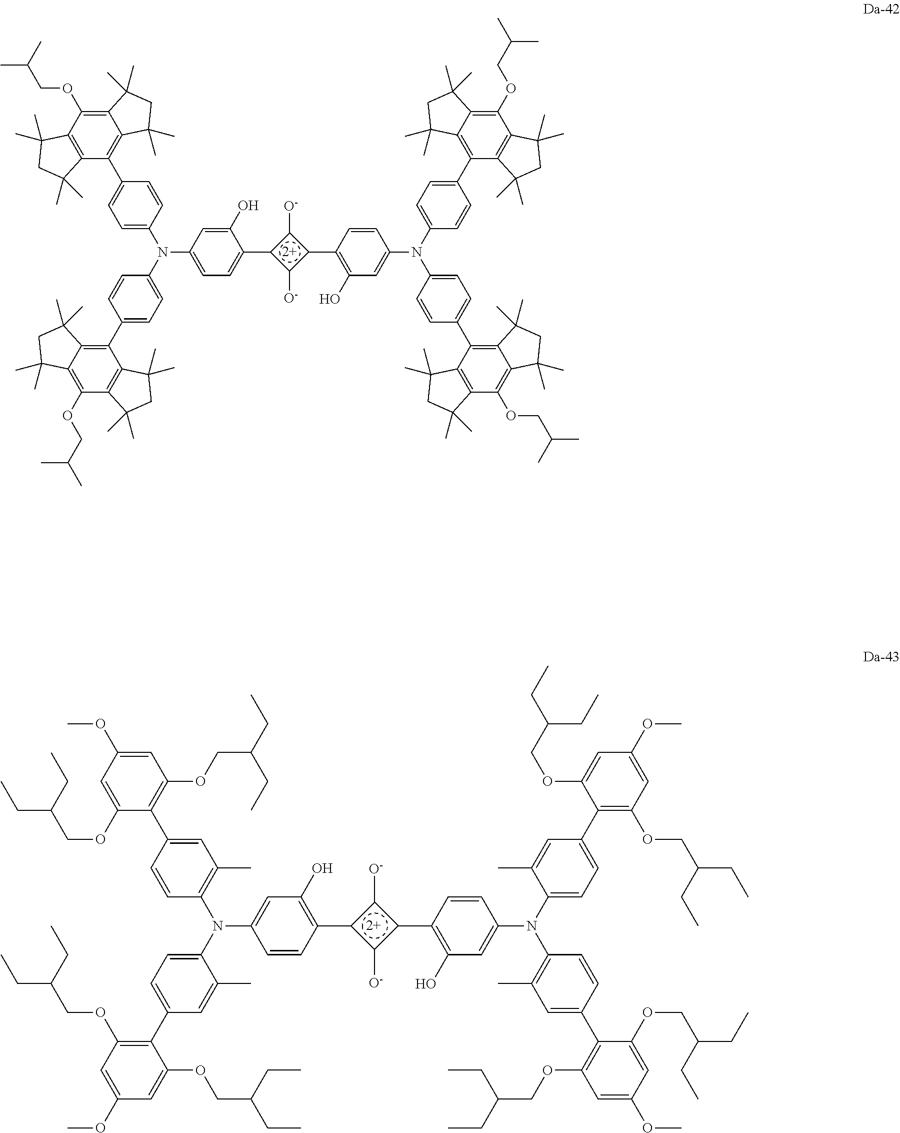

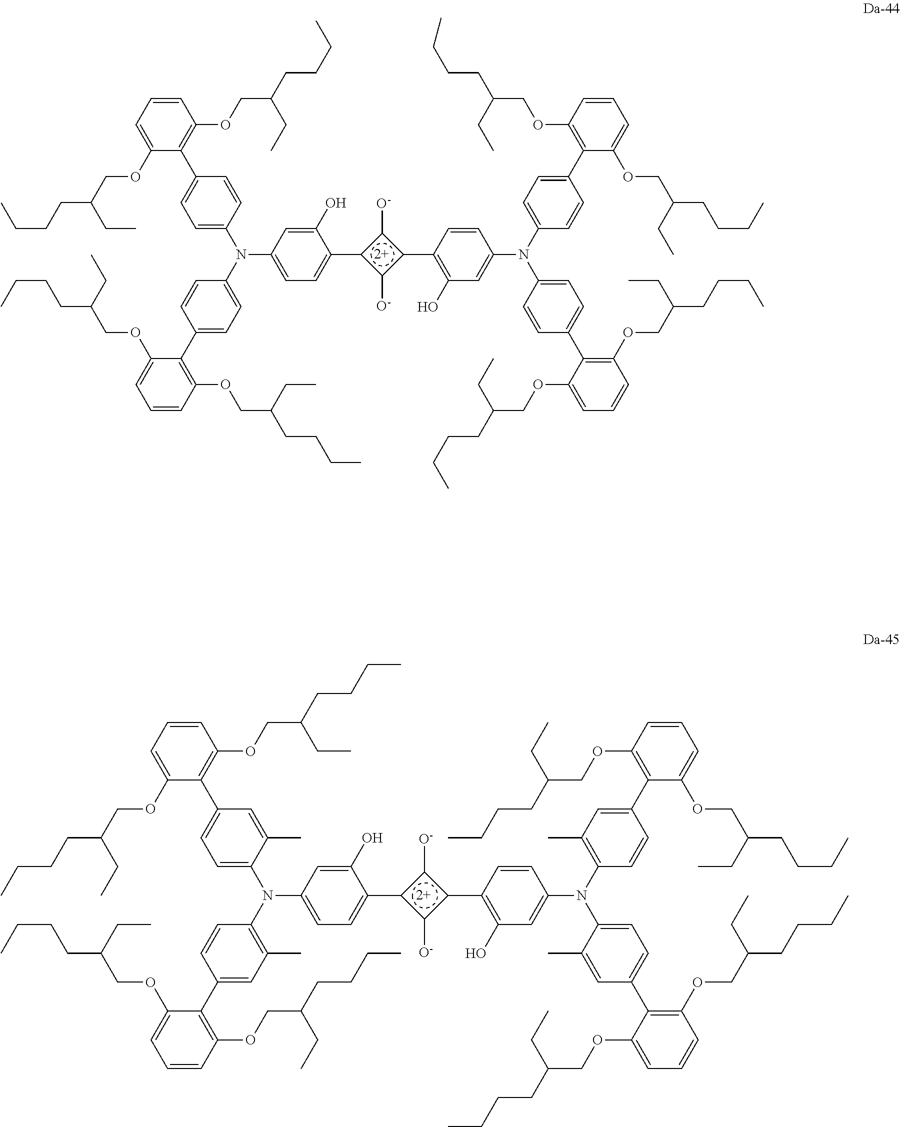

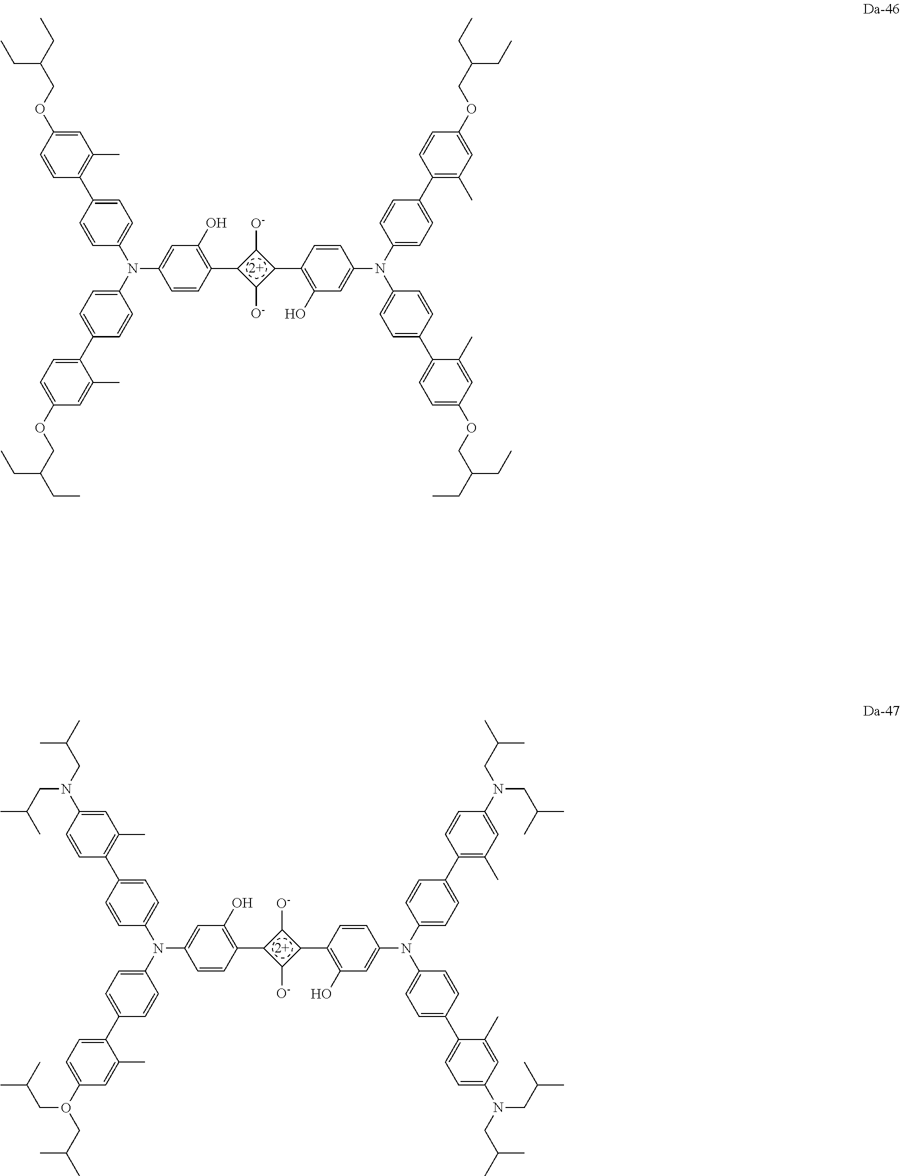

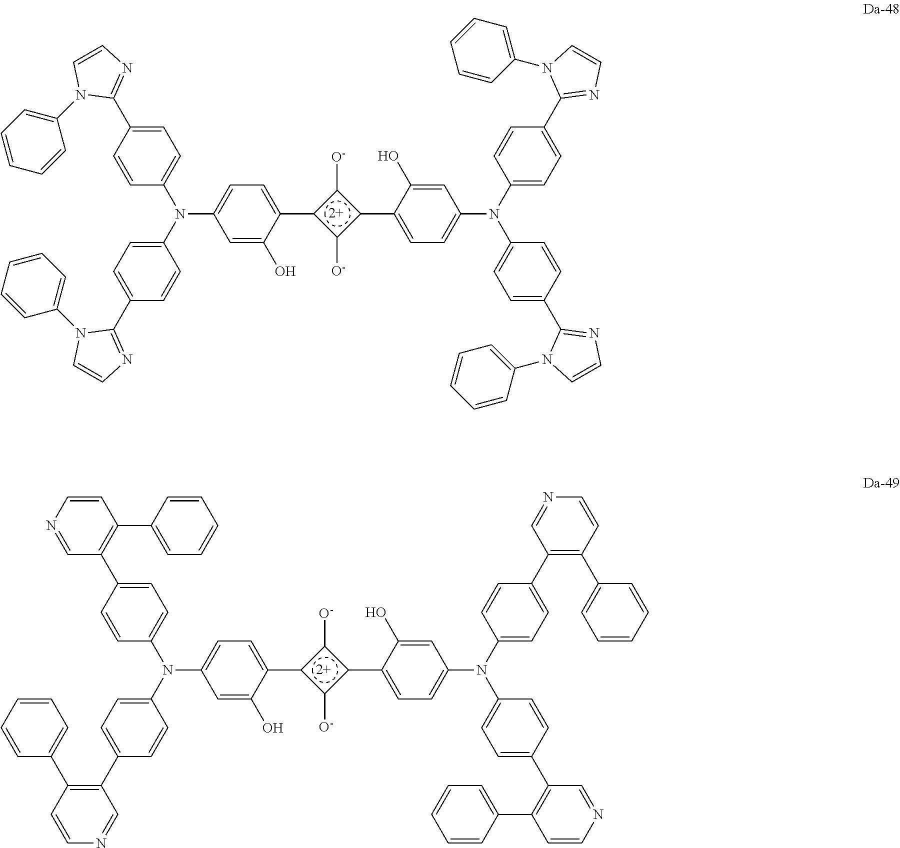

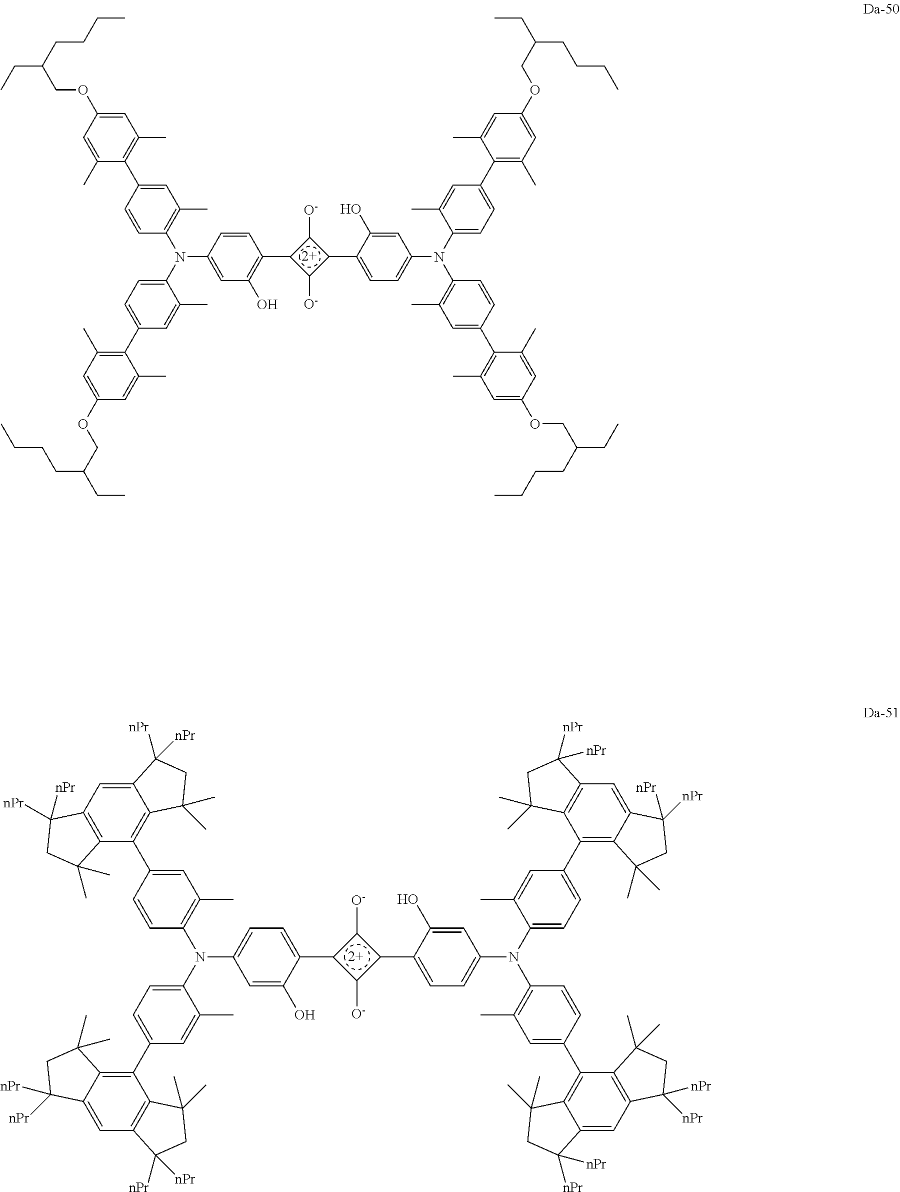

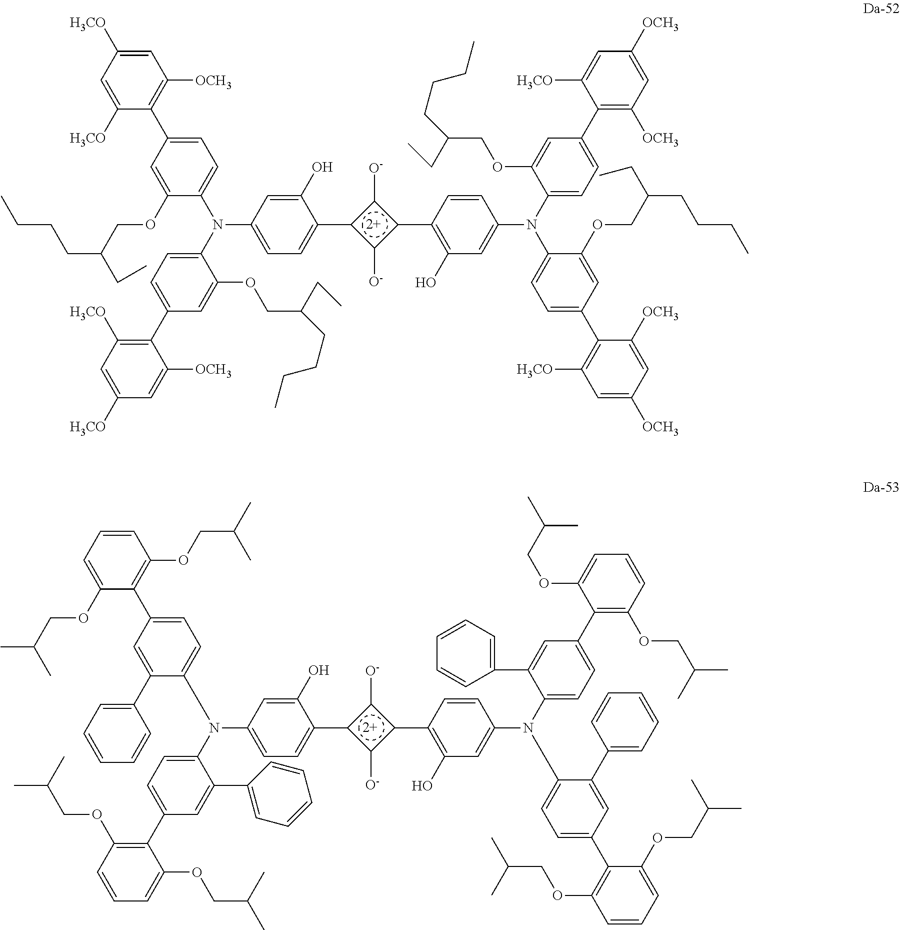

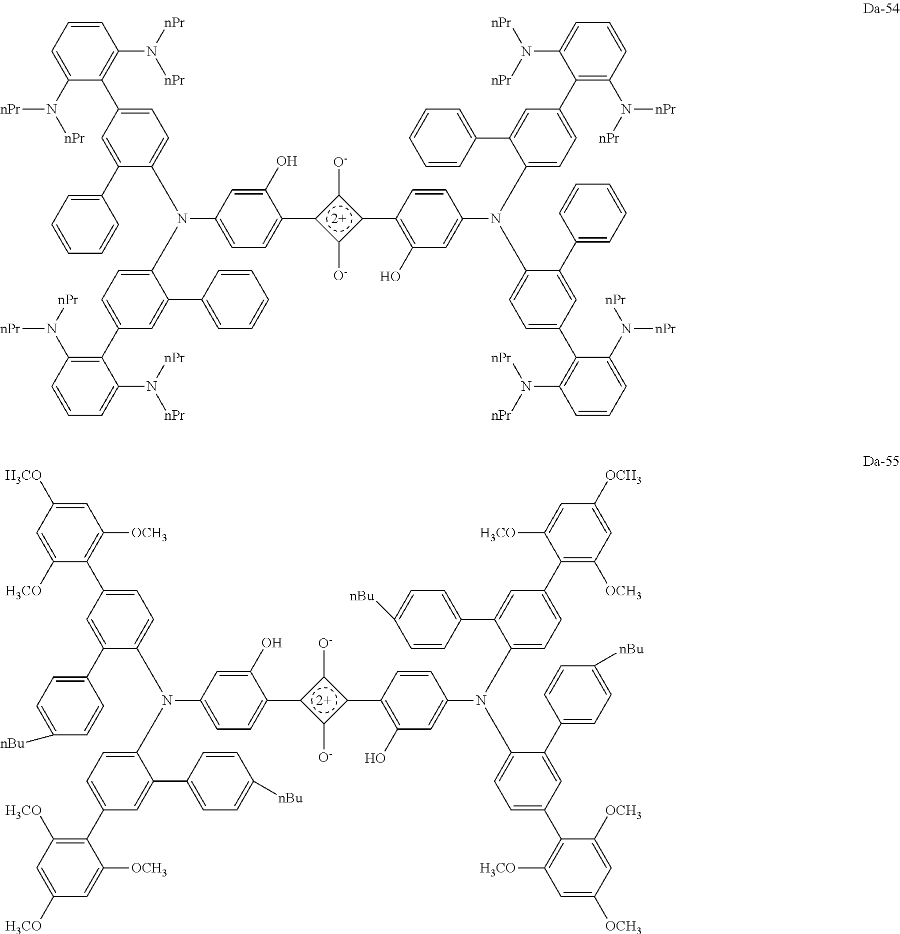

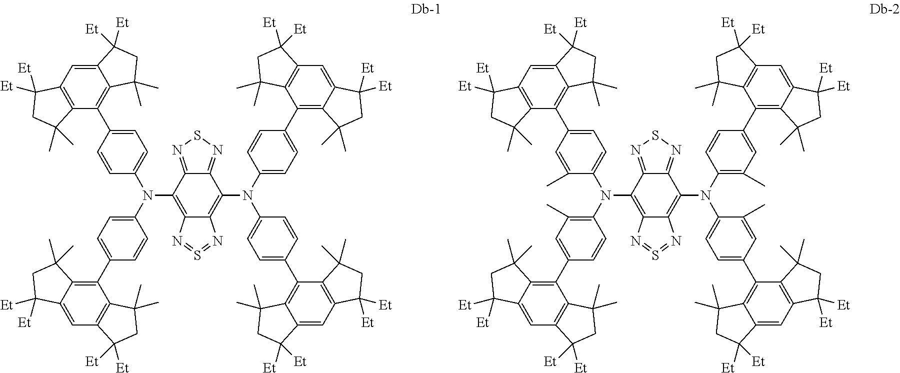

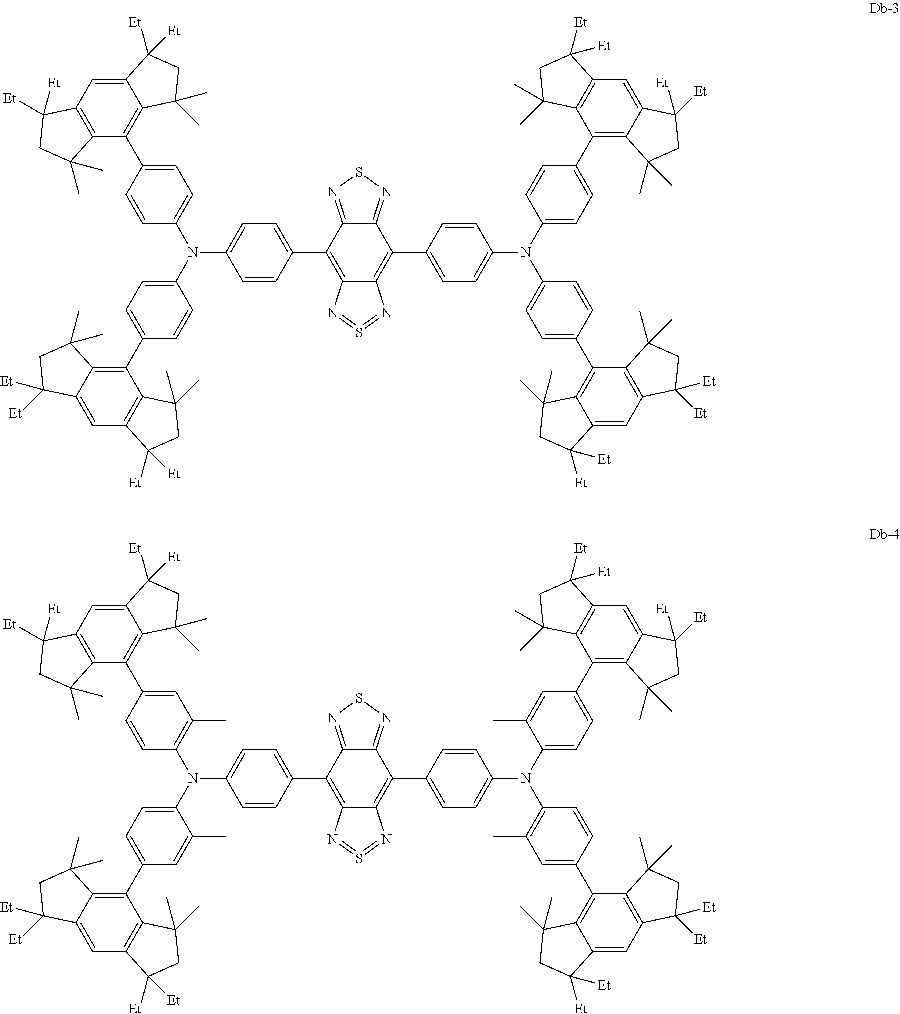

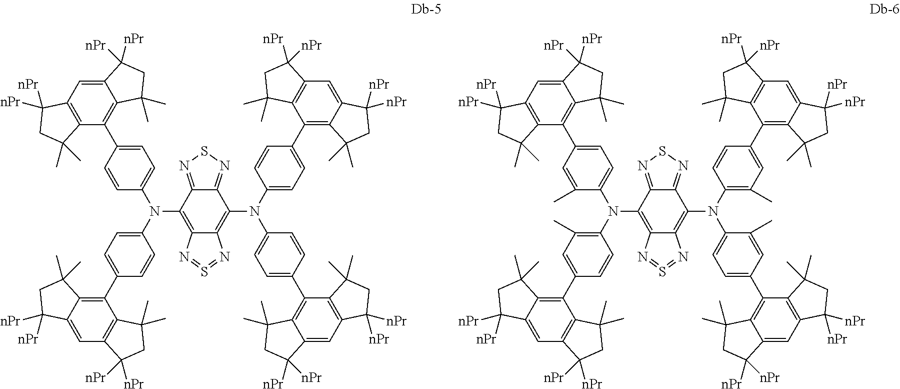

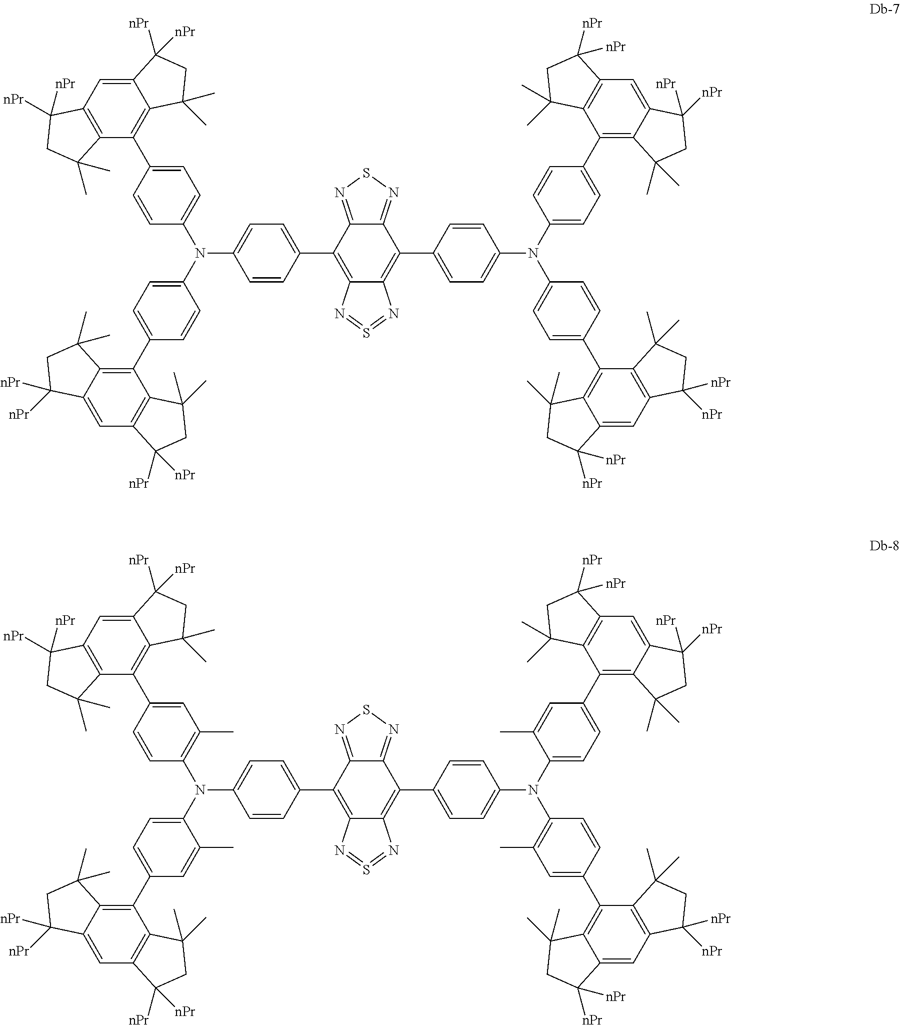

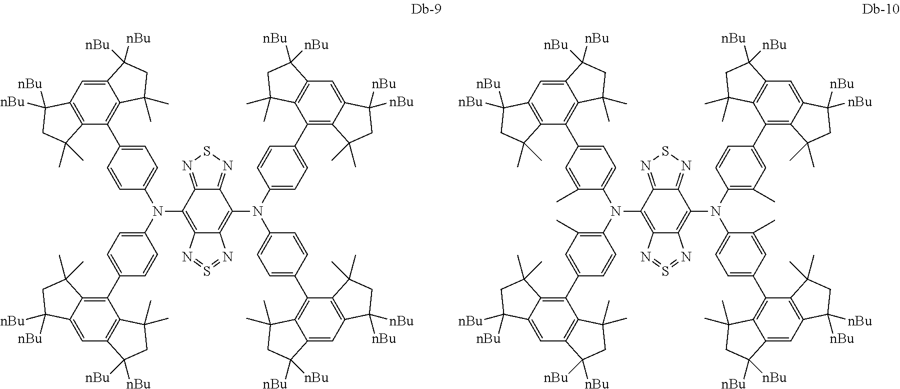

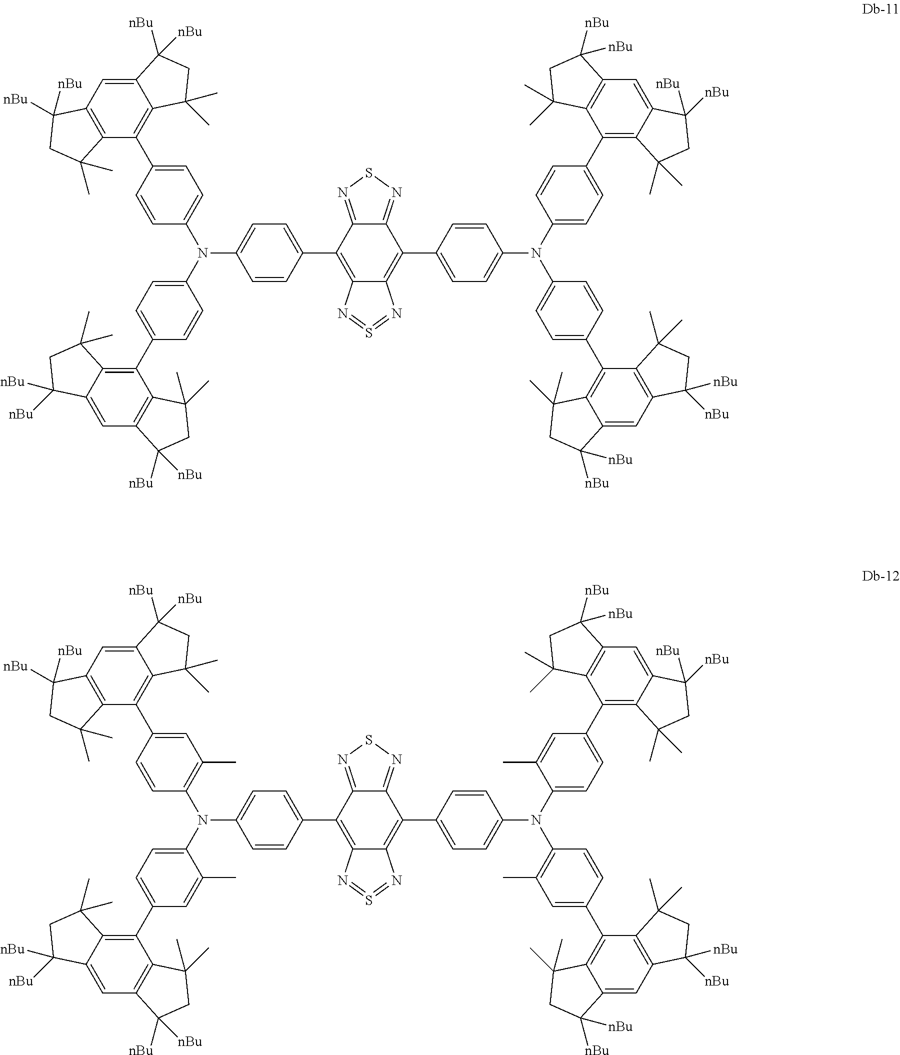

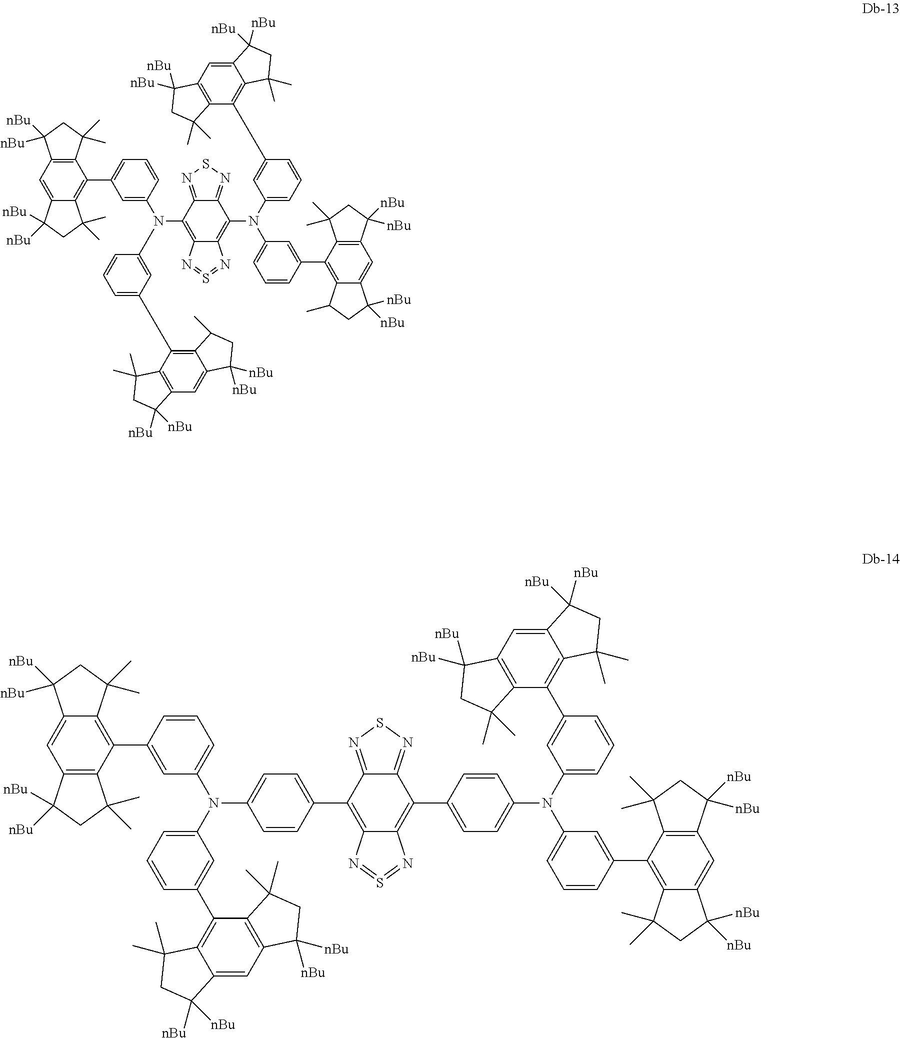

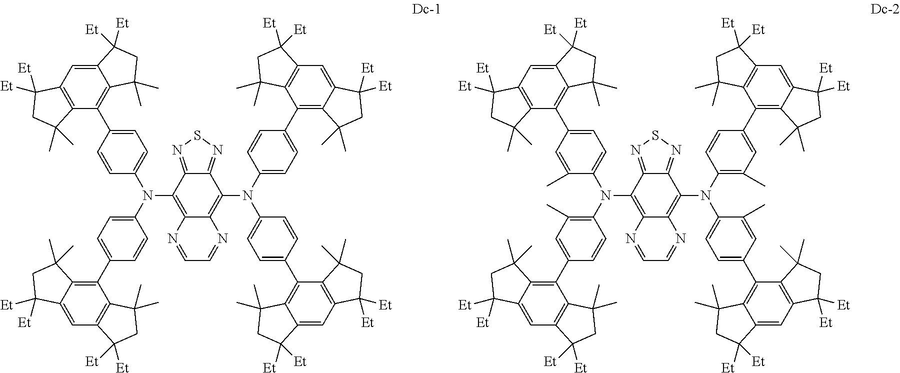

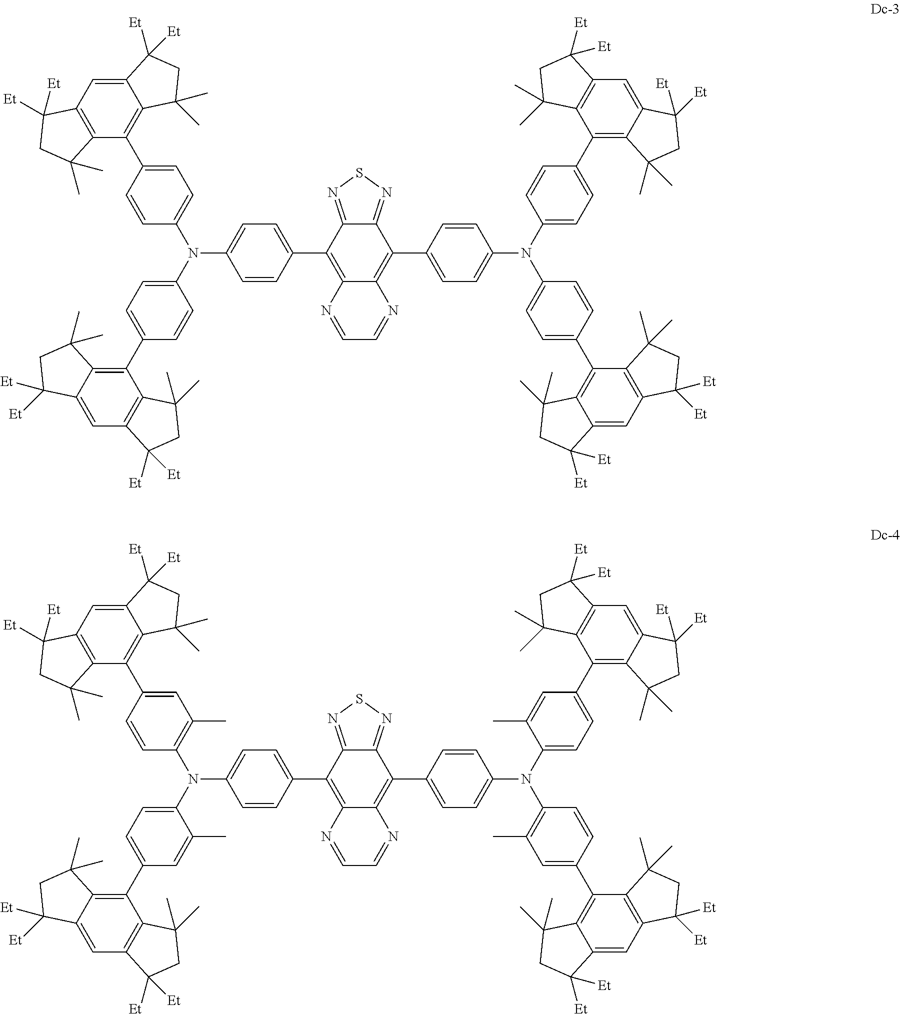

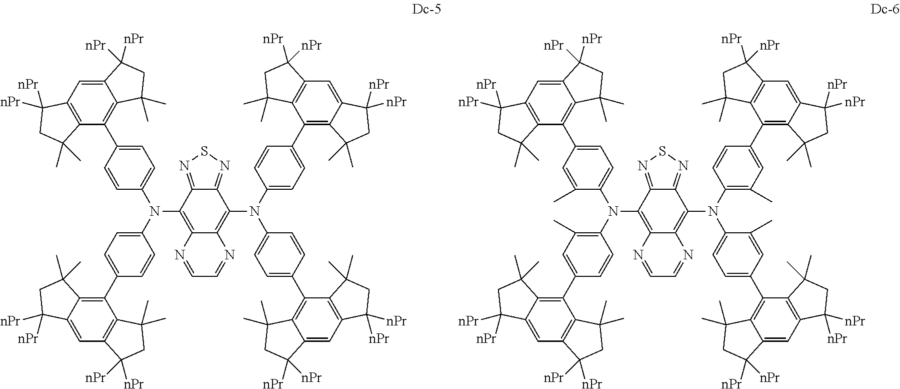

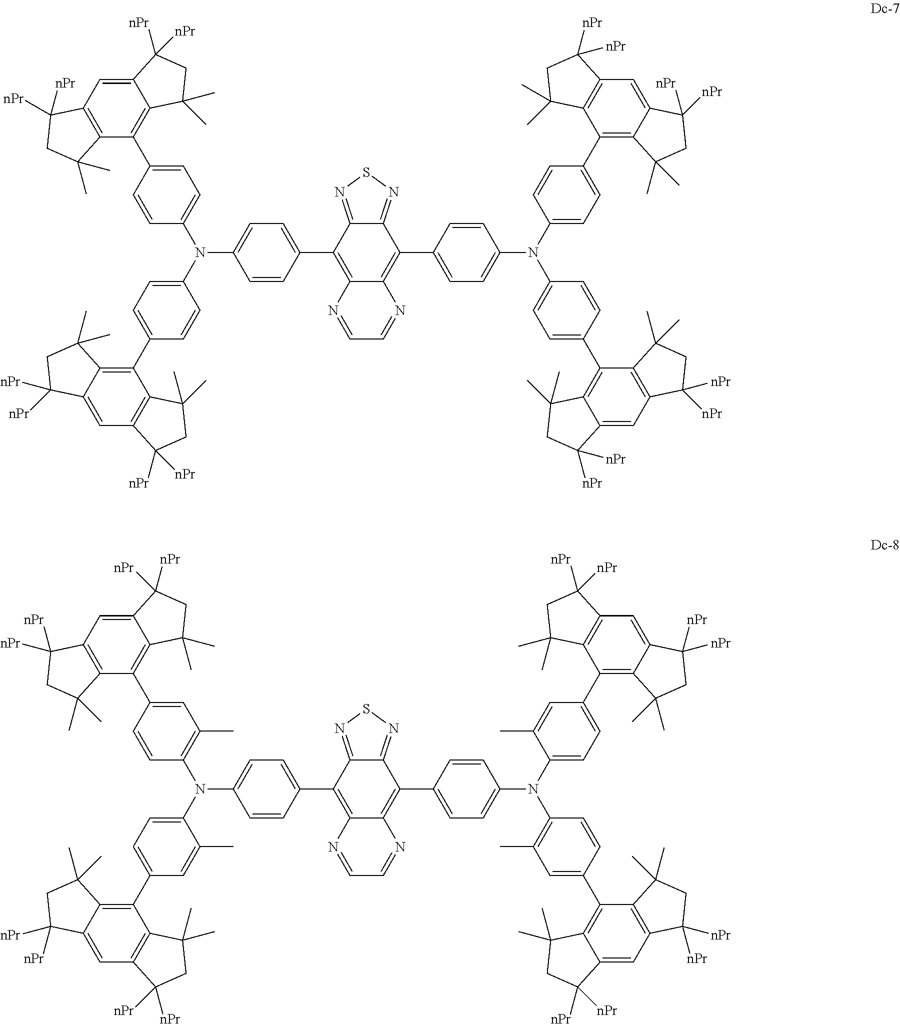

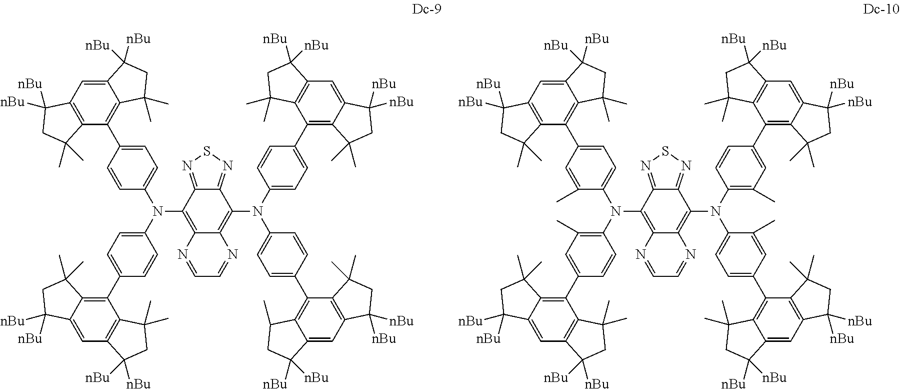

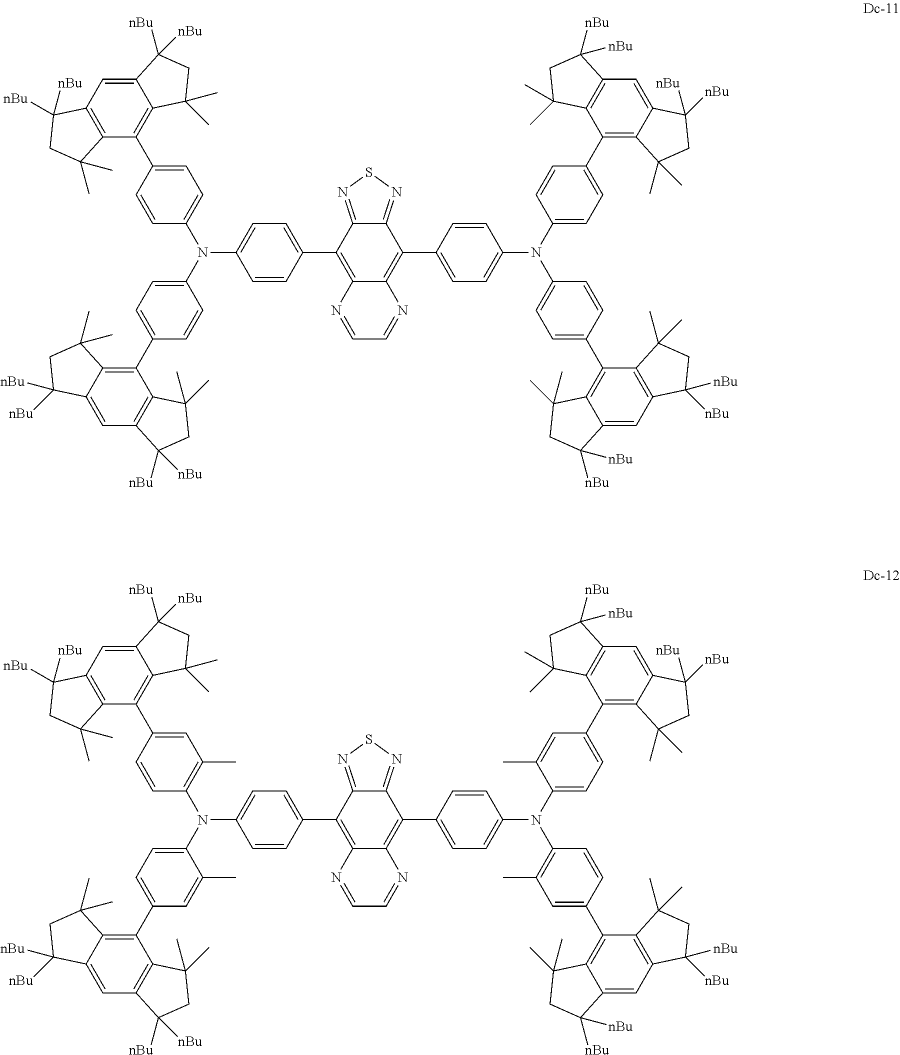

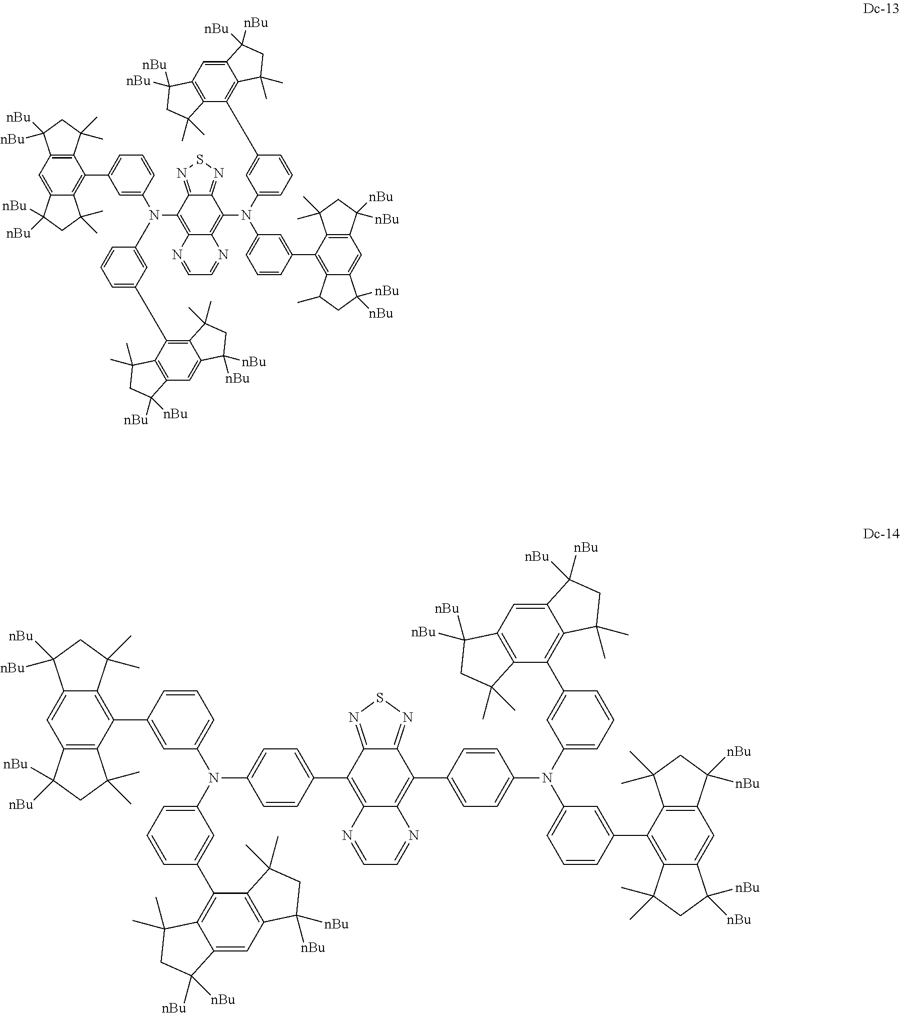

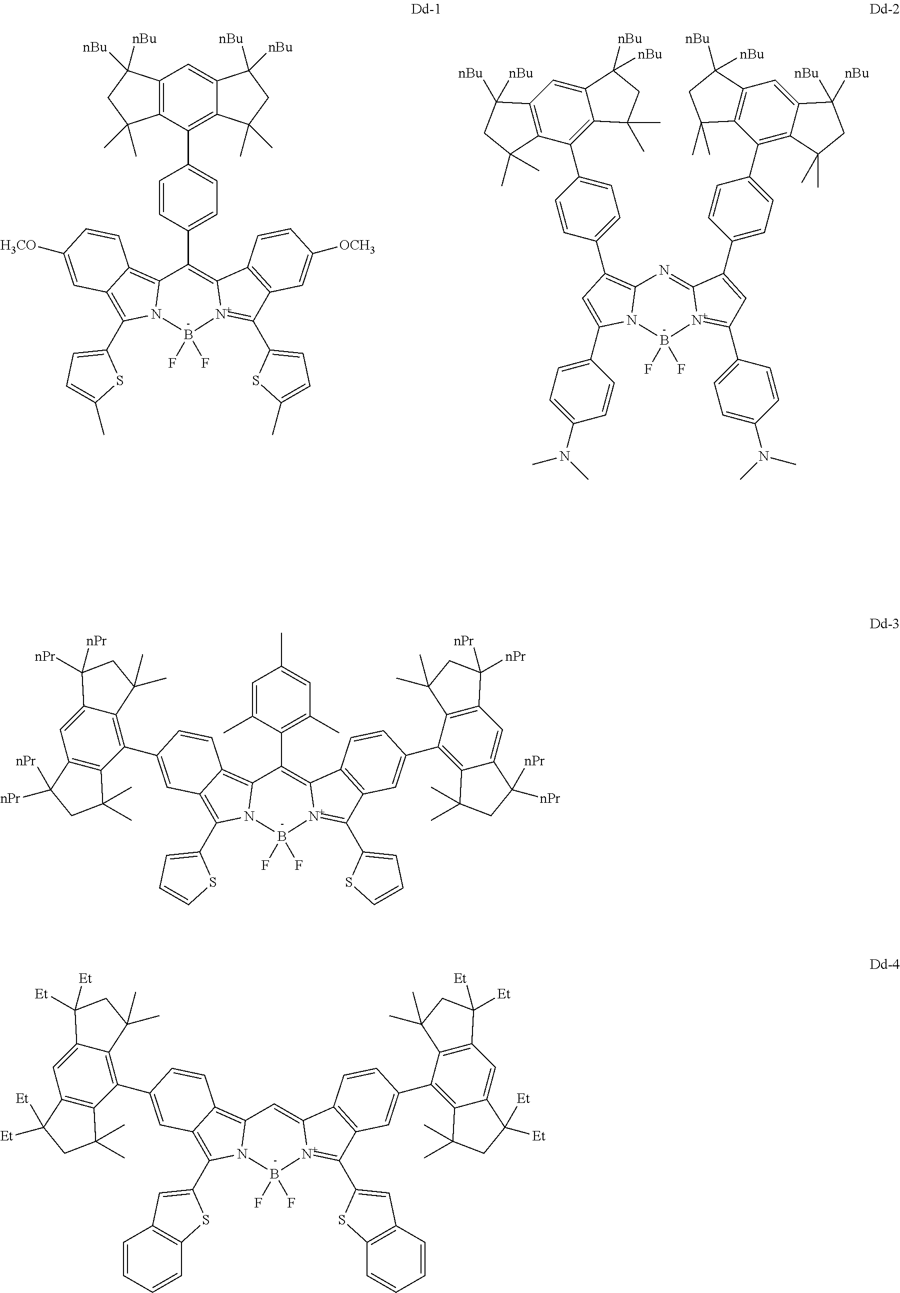

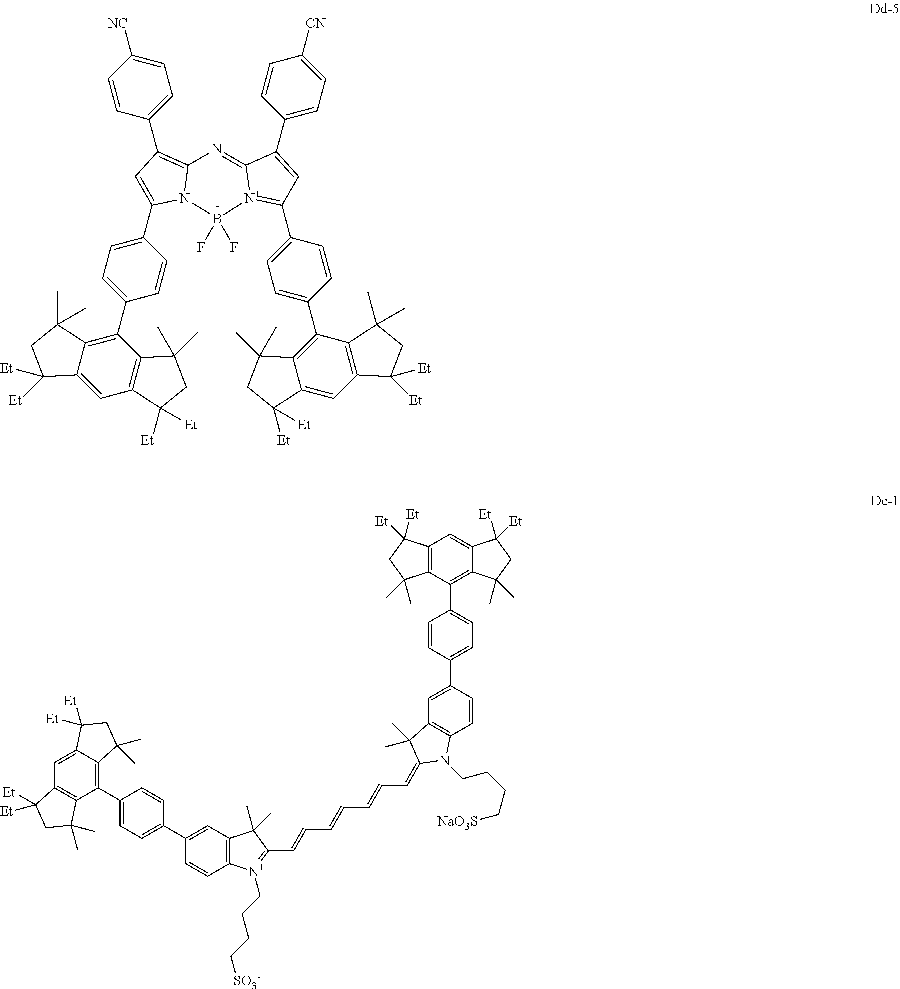

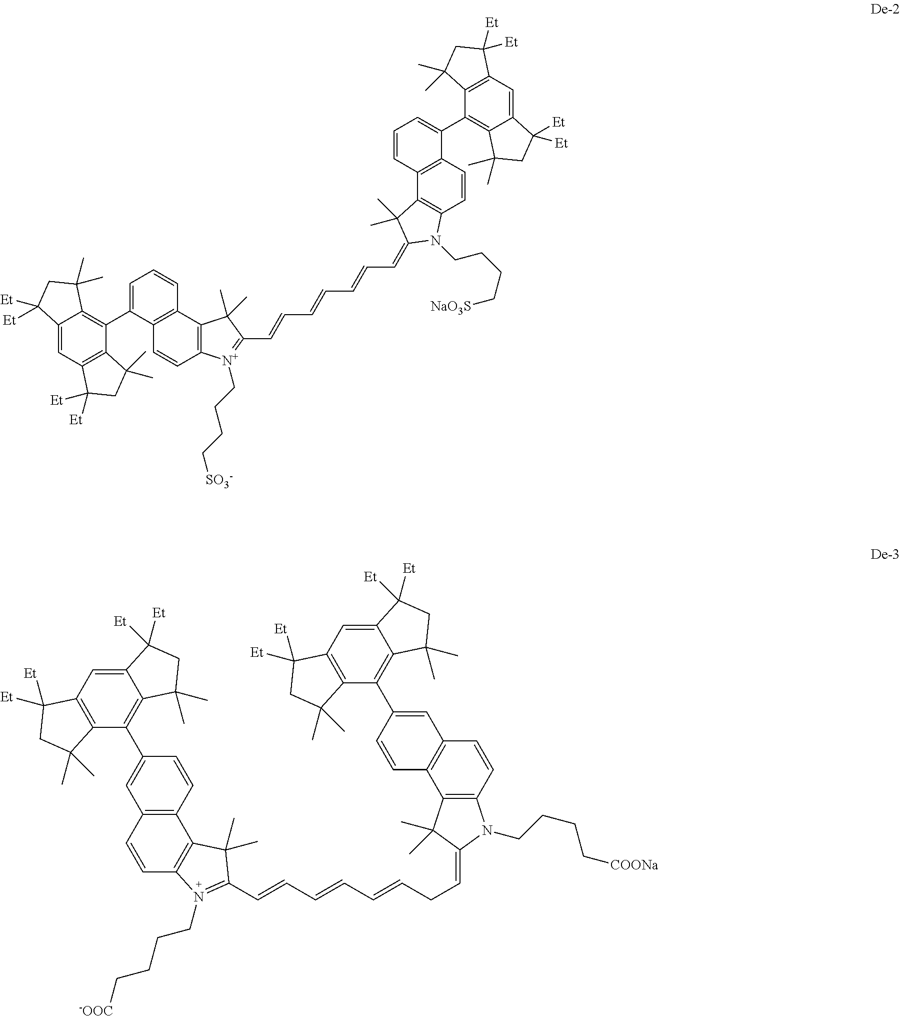

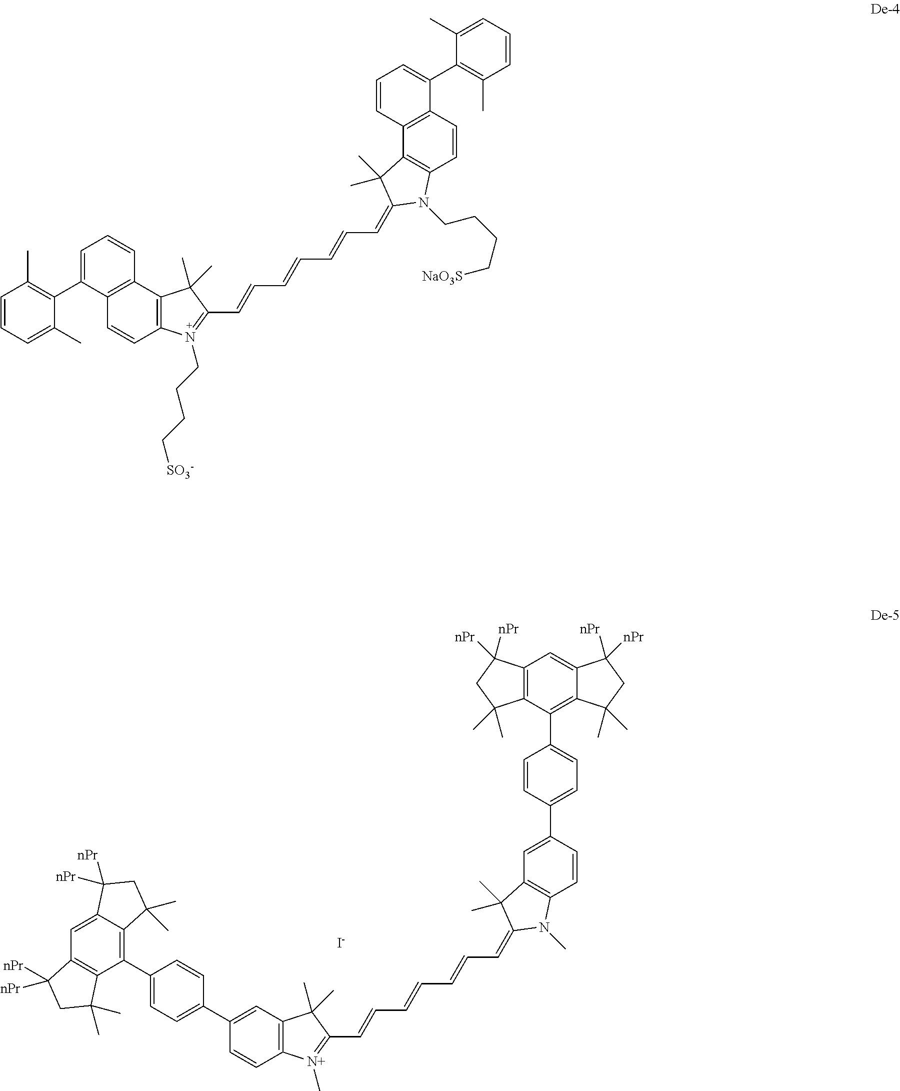

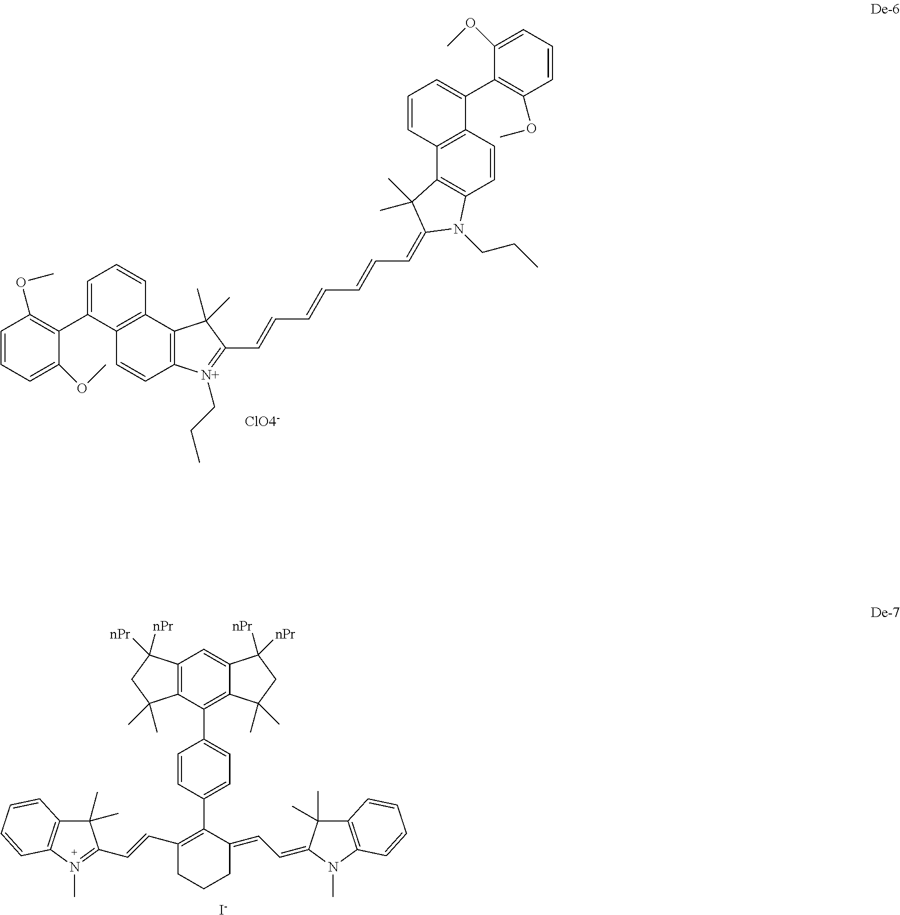

### <<Biometric Authentication Device>>

As an authentication device that authenticates an object using various technologies, the following devices are cited: an image pickup device that reads and authenticates an image, a mobile device such as a mobile phone equipped with the image device, an automated cash trading machine, a display, a facsimile, a scanner, and a multifunction device. In the information-oriented society in recent years, since high-precision authentication technology is required, research on biometrics is becoming active. In biometric authentication, an individual is recognized by collecting the biological characteristics (movements and a part of the living body) of each individual and measuring and determining the similarity with the characteristic data registered in advance.

Biological information includes veins of palms and fingers, fingerprints, palm shapes, irises, retinas, faces, handwritings, sounds, and odors. Among them, authentication using veins (vein authentication device) has attracted particular attention because of its small size, no risk of theft, and high security.

Vein authentication is an authentication method that utilizes the property of hemoglobin flowing in a vein that absorbs near-infrared light. The light source used in the authentication device is near-infrared light, and it is more preferable that the light source has a maximum emission in the vicinity of 850 nm. Vein authentication is performed by irradiating the fingertip with near-infrared rays and collating the obtained image in which the vein portion is shaded with the data registered in advance. Since the amount of data to be extracted is small in the vein authentication method, high-speed processing is possible, and visual confirmation is possible only after irradiating with near-infrared light. Therefore, forgery and theft are less likely to occur as compared with fingerprint authentication, which is a technique for authenticating the same part of a living body. Further, since the vein pattern is information existing inside the living body, it is not easily affected by the outside and does not change semi-permanently, and there are very few maladaptated persons.

A specific configuration of the vein recognition device includes, for example, a light source that emits near-infrared light, a control unit that adjusts the light source, and an imaging unit that detects transmitted light or reflected light from a fingertip to obtain an image,

an authentication unit that performs image processing, a storage unit that stores and registers extracted data, and a calculation unit that collates with registered data.

In the current vein authentication, irradiation is generally performed using a plurality of point light sources such as light emitting diodes (LEDs). However, since the LED is a point light source having a spherical light emitting portion, the brightness becomes uneven when a plurality of LEDs are arranged, and the recognition rate of the obtained image pickup is lowered. Further, since the individual light amounts of LEDs are not strictly uniform in the first place, a complicated system is required for a control unit that adjusts the irradiation light amount. Therefore, in the biometric authentication device of the present invention, it is preferable to use a surface light source having luminance uniformity, more specifically, it is preferable to use an OLED as the light source. When the surface light source is used as a vein authentication device, for example, a rectangular shape of 40 mm×10 mm is preferable.

The biometric authentication device of the present invention may be mounted on various devices, and one of the features is that it is mounted on a wristband type electronic device.

The wristband type electronic device of the present invention is not particularly limited, and examples thereof include a bracelet, a wristwatch, a smart watch, and a wristwatch type smartphone. They are devices capable of transmitting biometric authentication information by wireless communication.

As a preferred embodiment of the wristband type electronic device of the present invention, the light source is provided in any region on the wristband other than the same plane as the imaging unit when the wristband type electronic device is worn. By providing the light source in any region on the wristband other than the same plane as the imaging unit at the time of wearing, it is possible to reduce the influence of biological information unnecessary for authentication by the reflected light on the biological surface. Since it is possible to receive biological information in the near-infrared light scattered in the living body, vein imaging is facilitated. The imaging portion of the wrist is not particularly limited, but vein imaging on the outside or inside of the wrist is suitable for authentication.

The position of the light source is not limited as long as it is in any region on the wristband other than on the same plane as the imaging unit when mounted. However, in order to further reduce the influence of biometric information unnecessary for authentication, the angle formed by the center point of the imaging unit, the center point of the light source, and the center point of the wrist cross section is preferably in the range of 30 to 180 degrees, more preferably in the range of 45 to 180 degrees, and still more preferably in the range of 90 to 180 degrees.

The number of light sources is not particularly limited, but it is preferable to install them in the range of 1 to 3 from the viewpoint of power consumption.

The imaging unit is not particularly limited, but it is preferable to use a wide-angle camera because the vein pattern is photographed at a wide angle.

### <<Biometric Device>>

Biometric devices are now an indispensable device for daily health management and medical practice in society. On the other hand, since it is necessary to attach the sensor to the body, compatibility with QOL (quality of life, also referred to as “quality of living”) has become an issue.

Since the light emitting member of the present invention emits light in the region of the “biological window”, it is suitable for mounting on a biometric measuring device.

The biometric device provided with the light emitting member of the present invention is not particularly limited, and examples thereof include a pulse oximeter and a pulse wave sensor. With a pulse oximeter, the oxygen concentration in blood may be measured using two wavelengths, near-infrared light and red light.

Since pulse oximeters and pulse wave sensors in medical settings are generally attached to a fingertip, they reduce the QOL of inpatients. Since the pulse oximeter and pulse wave sensor provided with the light emitting member of the present invention use surface light sources, it enables sensing at thick bones (a wrist and base of a finger), which is difficult with conventional point light sources, and fingertips. It is possible to eliminate the troublesomeness of the fingertip. The reason is not clear, but it is speculated as follows.

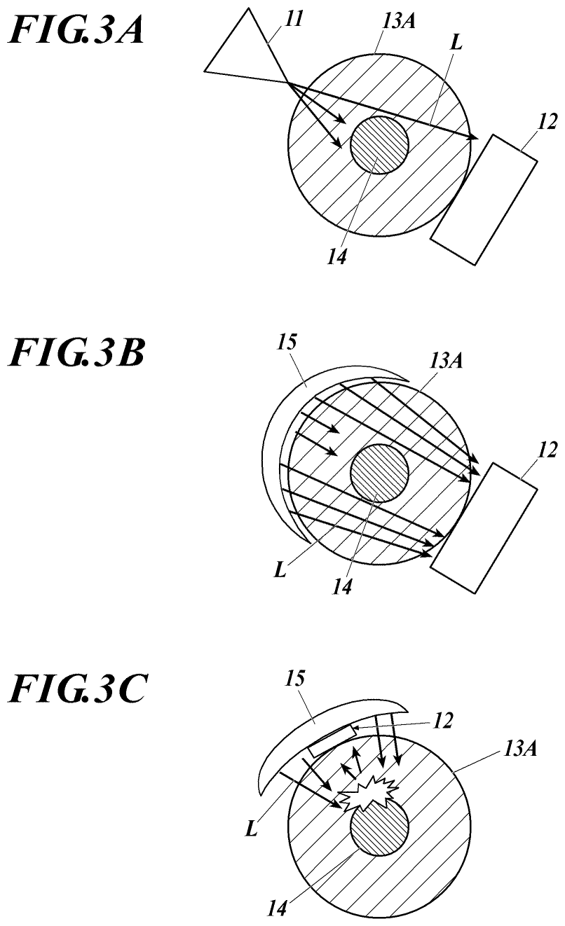

FIG. 3A to FIG. 3C each are a schematic view showing an example of sensing at a fingertip using a point light source and a surface light source as a light emitting member.

FIG. 3A shows the transmissive sensing of a fingertip by the conventional point light source 11, and since the bone 14 is thin at the tip portion 13A of the finger, even the point light source 11 passes through the living body, the light L is transmitted and achieves to the sensor 12.

FIG. 3B shows the transmissive sensing of a fingertip using the surface light source 15 of the present invention. The surface light source 15 of the present invention has a large light emitting area and the directivity of the light L is not strong. Therefore, the restriction on the alignment between the surface light source 15 and the sensor 12 is remarkably reduced, and the light L stably passes through the living body and reaches the sensor 12.

FIG. 3C shows the reflective sensing of a fingertip using the surface light source 15 of the present invention. The surface light source 15 of the present invention has a large light emitting area and the directivity of the light L is not strong. Therefore, the scattering of the light L in the living body is promoted, and the light L is stably reflected in the living body and reaches the sensor 12.

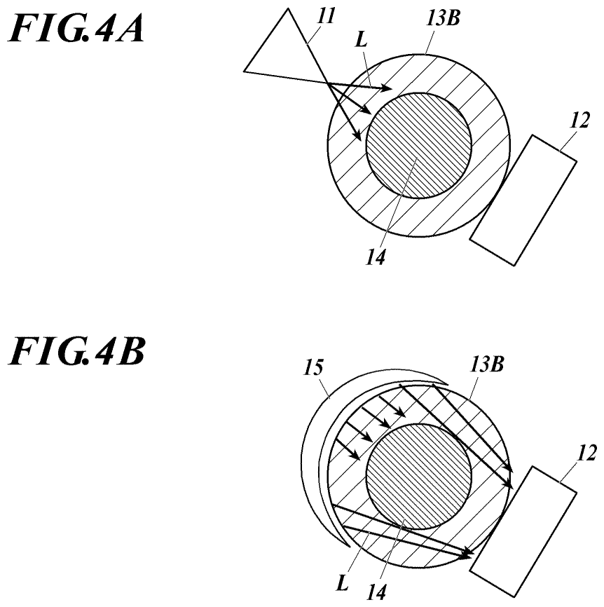

FIG. 4A and FIG. 4B each are a schematic view showing an example of sensing at the base of a finger using a point light source and a surface light source as light emitting members. FIG. 4A shows sensing using a conventional point light source 11. As shown in FIG. 4A, at the base of the finger 13B, the conventional point light source 11 has a small light emitting area and strong directivity of light L. Therefore, the light L is often blocked by the bone 14, and the alignment between the point light source 11 and the sensor 12 is limited to a very narrow range, which makes stable sensing difficult.

On the other hand, in the case of sensing at a base of a finger using the surface light source 15 of the present invention, as shown in FIG. 4B, the light emitting area is large and the directivity of the light L is not strong. Therefore, the restriction on the alignment between the surface light source 15 and the sensor 12 is remarkably reduced, and the light L stably passes through the living body and reaches the sensor 12.

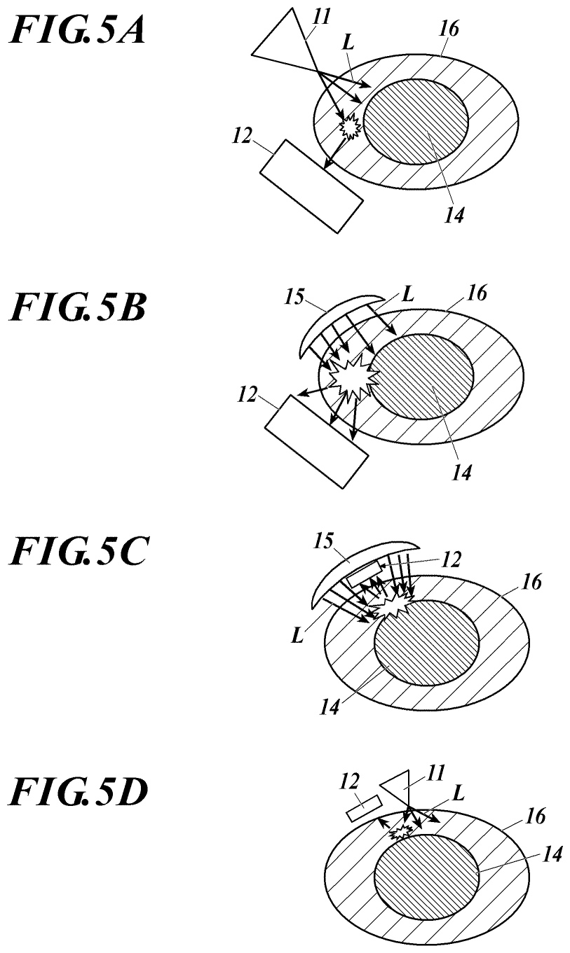

FIG. 5A to FIG. 5D each are a schematic view showing an example of sensing on a wrist using a point light source and a surface light source.

A conventional point light source 11 is used for the V-shaped sensing in FIG. 5A and the reflective wrist sensing in FIG. 5D. Since the directivity of the light L is strong in these sensing, there is a problem that the light L is not sufficiently scattered in the living body and a sufficient amount of light for sensing cannot be received. On the other hand, in the sensing on the V-shaped wrist shown in FIG. 5B, since the surface light source 15 which is the light emitting member of the present invention is used, light L enters the living body over a wide area, and light scattering in the living body becomes active. It is possible to obtain biological information with the amount of light required for sensing. Similarly, the same effect as that of FIG. 5B may be obtained in the reflex type wrist sensing shown in FIG. 5C.

### EXAMPLES

Hereinafter, the present invention will be specifically described with reference to Examples, but the present invention is not limited thereto. Unless otherwise specified, “%” and “part” mean “mass %” and “part by mass”, respectively.

### Example 1

### <<Preparation of Organic EL Elements>>

### [Preparation of an Organic EL Element A]

According to the following procedure, anode/hole injection layer/hole transport layer/light emitting layer/electron transport layer/electron injection layer/cathode are laminated in this order on the substrate and sealed. Thus a bottom emission type organic EL element A was produced.

### (Formation of an Anode)

First, on the entire surface of the side of the polyethylene naphthalate film (manufactured by Teijin DuPont Co., Ltd., hereinafter abbreviated as PEN) that forms an anode, an inorganic gas barrier layer made of SiOx was formed so as to have a thickness of 500 nm by using an atmospheric pressure plasma discharge processing apparatus having the configuration described in JP-A-2004-68143. As a result, a flexible base material (gas barrier film) having a gas barrier property with an oxygen permeability of 0.001 mL/(m²·24 h·atm) or less and a water vapor permeability of 0.001 g/(m²·24 h) or less was prepared.

Next, ITO (In₂O₃:SnO₂=90:10 mass % ratio) was formed by a sputtering method so that the thickness was 150 nm, and then patterning was performed to form an anode. The pattern had an area of a light emitting region of 10 mm×40 mm. Subsequently, after ultrasonic cleaning with isopropyl alcohol, the anode was dried with dry nitrogen gas, and UV ozone cleaning was performed for 5 minutes.

### (Formation of a Group of Organic Functional Layers)

The anode produced by the above method was dried in a glove box having a dew point of −80° C. or less and an oxygen concentration of 1 ppm or less, and then transferred into a vacuum vapor deposition apparatus. The crucible (made of molybdenum or tungsten, which is a material for resistance heating) in the vacuum vapor deposition apparatus was filled with the constituent materials of each of the organic functional layers described below in an amount required for producing an organic EL element.

### <Formation of a Hole Injection Layer>

The vacuum vapor deposition apparatus was depressurized to 1×10⁻⁴ Pa, and the compound HIL-1 (MTDATA) was vapor-deposited at a vapor deposition rate of 0.1 nm/sec, and a hole injection layer having a thickness of 15 nm (hereinafter referred to as HIL) was formed.

### <Formation of a Hole Transport Layer>

Next, the following compound HTL-1 (α-NPD) was deposited on the HIL to form a hole transport layer (HTL) having a thickness of 30 nm.

### <Formation of a Red Phosphorescent Light Emitting Layer>

Subsequently, the heating boats each containing the light emitting host compound H-1 and the dopant compound DP-1 were independently energized, and the vapor deposition rates of H-1 and DP-1 were adjusted to be 100:6. Then, a red phosphorescent light emitting layer (EML) having a thickness of 20 nm was formed on the HTL.

### <Formation of an Electron Transport Layer>

Next, the heating boat containing tris(8-hydroxyquinolinate)aluminum (Alq₃) was energized and heated to be vapor-deposited at a vapor deposition rate of 0.1 nm/sec, and an electron transport layer (ETL) having a thickness of 25 nm was formed on the EML.

### <Formation of an Electron Injection Layer>

Next, LiF was deposited at a vapor deposition rate of 0.1 nm/sec to form an electron injection layer (EIL) having a thickness of 1 nm on the ETL.

### (Formation of a Cathode)

Subsequently, aluminum was deposited to a thickness of 70 nm to form a cathode on the EIL.

### (Formation of a Sealing Structure)

Next, a sealing base material was adhered onto the cathode of the above-mentioned laminate formed from the anode to the cathode using a commercially available roll laminating device. The sealing base material was prepared as follows. A flexible aluminum foil (manufactured by Toyo Aluminum K.K. Co., Ltd.) with a thickness of 30 μm was provided with an adhesive layer having a thickness of 1.5 μm using a two-component reaction type urethane adhesive for dry lamination. Then, a 12 μm polyethylene terephthalate (PET) film was laminated thereon to obtain the sealing base material.

A thermosetting adhesive was uniformly applied along the adhesive surface (glossy surface) of the aluminum foil of the sealing base material using a dispenser to form an adhesive layer having a thickness of 20 μm. This was dried under a reduced pressure of 100 Pa or less for 12 hours. As the thermosetting adhesive, a mixture of the following components (A) to (C) was used.

(A) Bisphenol A diglycidyl ether (DGEBA)

(B) Dicyandiamide (DICY)

(C) Epoxy adduct-based curing accelerator

Next, the sealing base material was transferred to a nitrogen atmosphere having a dew point temperature of −80° C. or less and an oxygen concentration of 0.8 ppm, dried for 12 hours or longer, and adjusted so that the water content of the sealing adhesive was 100 ppm or less.

Finally, the sealing base material was closely adhered and arranged with respect to the laminate, and was tightly sealed using a pressure-bonding roll under the conditions of a temperature of 100° C. with a pressure of 0.5 MPa, and an apparatus speed of 0.3 m/min. Then, the adhesive was cured by subjecting it to heat treatment at 110° C. for 30 minutes to obtain an organic EL element A.

The details of each constituent material used for producing the organic EL element A are as follows.

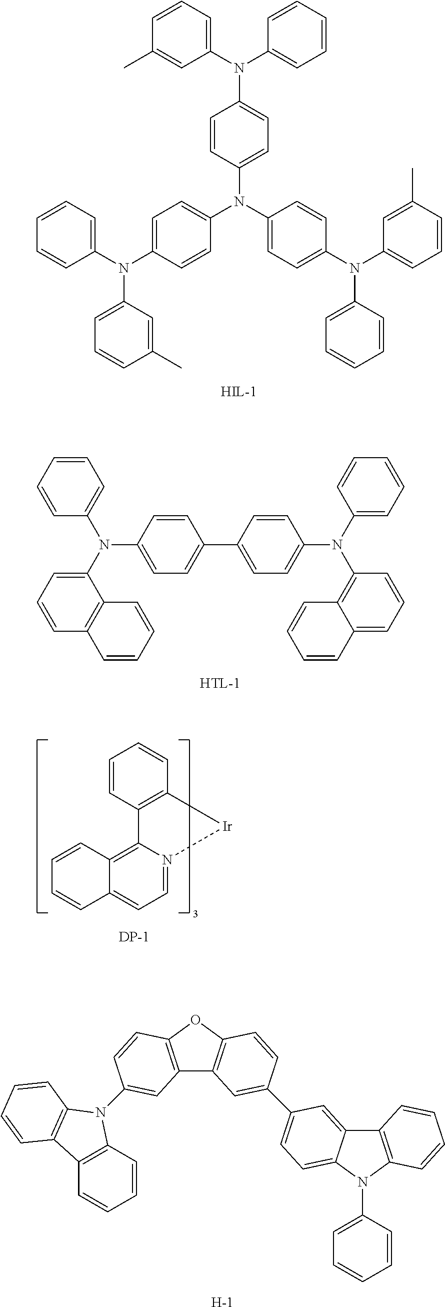

### (Measurement of Brightness Uniformity)

The brightness uniformity of the organic EL element A, which is a surface light source produced by the above method, was measured according to the following method.

The measurement was performed using a luminance meter (CS-1000) manufactured by Konica Minolta, Inc. according to the method described in JP-A2010-80473. The organic EL element A was driven under the condition that the brightness of the light source was 1000 cd/cm², the brightness at 10 places was randomly measured, and the brightness uniformity was evaluated by the following Equation (1).

Brightness uniformity (%)=[Minimum brightness on the light emitting surface (cd/cm²)/Maximum brightness on the light emitting surface (cd/cm²)]×100 Equation (1)

The brightness uniformity of the organic EL element A measured by the above method was 93%.

### [Preparation of an Organic EL Element B]

An organic EL element B was produced in the same manner as preparation of the organic EL element A except that the following were changed: the material for forming the hole injection layer (HIL) was changed from HIL-1 to the following HIL-2; the material for forming the hole transport layer (HTL) was changed from HTL-1 to the following HTL-2; the red phosphorescent light emitting layer (EML) was formed by co-depositing the following H-2, Dp-2 and Dp-3 in a mass ratio of 75:24:1; and the material for forming the electron transport layer (ETL) layer was changed from ETL-1 to ETL-2 and ETL-3.

The details of each constituent material used for producing the organic EL element B are as follows.

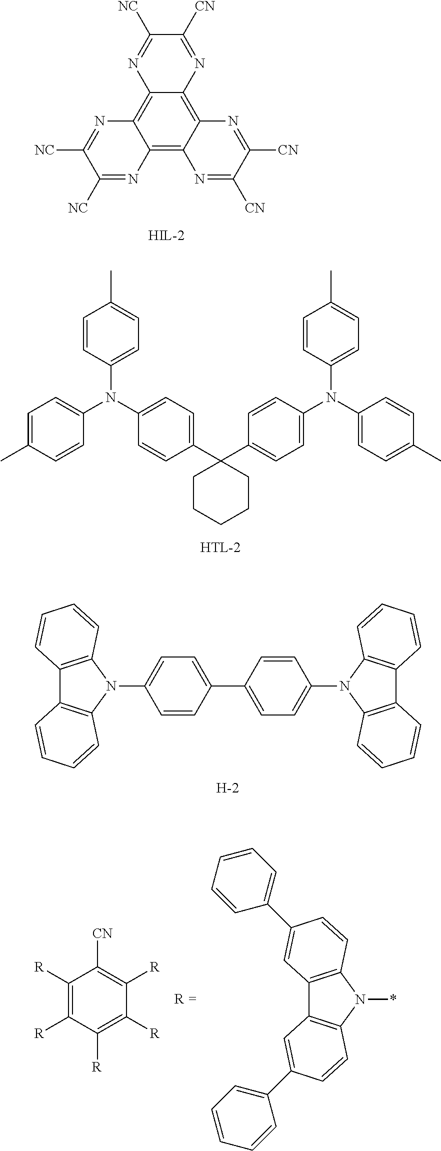

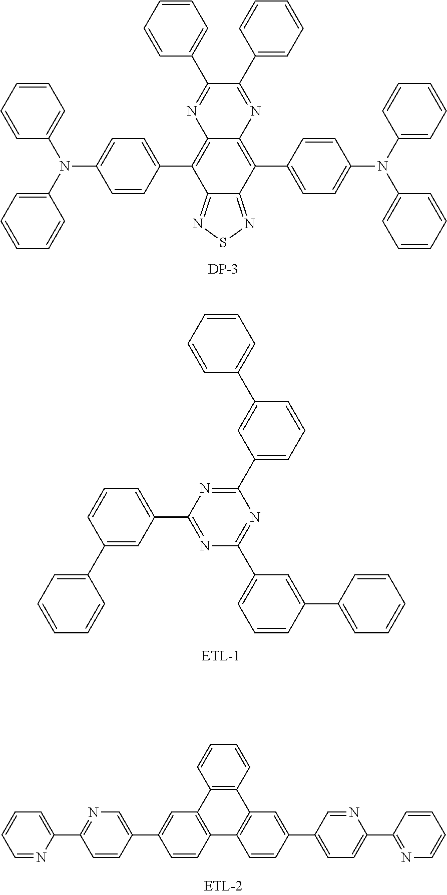

### [Preparation of an Organic EL Element C]

In the production of the organic EL element A, after forming the anode, a 2 mass % dispersion of poly (3,4-ethylenedioxythiophene)/polystyrene sulfonate (PEDOT/PSS) prepared in the same manner as in Example 16 of Japanese Patent No. 4509787 diluted with isopropyl alcohol was applied on the anode by spin coating method. It was dried at 80° C. for 5 minutes to form a hole injection layer having a thickness of 40 nm. Then, a solution (liquid concentration 0.4 mass %) in which the following luminescent dyes A-1, DP-1, and H-1 were dissolved in tetrahydrofuran (THF) so as to have a mass ratio of 0.5:15:84.5 was applied by a spin coating method (1500 rpm, 30 seconds) and dried at 50° C. for 30 seconds to form EML. After that, in the same manner as the organic EL element A, from the electron transport layer (ETL) layer to the sealing structure were formed to result in producing an organic EL element C.

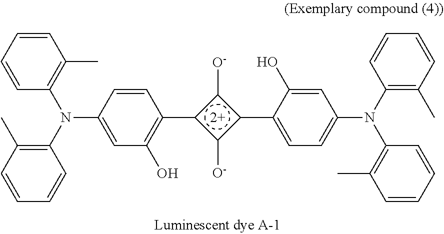

### <<Preparation of Wavelength Conversion Films>>

### [Preparation of a Wavelength Conversion Film 1-1]

A wavelength conversion film 1-1 having the configuration shown in FIG. 2 was prepared according to the following method.

A matrix material polystyrene (manufactured by ACROS ORGANICS Co., Ltd., weight average molecular weight Mw=260000) and luminescent dye A-1 (exemplary compound (4) above) were added to toluene as a solvent in a mass ratio of 99:1. These were placed in an eggplant flask and heated and stirred at 80° C. to sufficiently dissolve them.

Next, the obtained mixed solution was applied onto a gas barrier film (4A) similar to that used as the base material of the organic EL element described above using an applicator, dried at room temperature for 10 minutes. Then it was further heated and dried at 80° C. for 10 minutes to obtain a luminescent dye layer (5). Then, a gas barrier film (4B, same as above) to which the sealing adhesive (6) was attached was prepared and bonded to the coated surface, and sealed using a vacuum laminator under heating conditions of 90° C. Then, the adhesive was cured by heat treatment at 110° C. for 30 minutes to prepare a wavelength conversion film 1-1 (3) having a film thickness of 40 μm.

### [Preparation of Wavelength Conversion Films 1-2 to 1-7]

Wavelength conversion films 1-2 to 1-7 were prepared in the same manner as preparation of the wavelength conversion film 1-1, except that the luminescent dye A-1 (exemplary compound (4)) was changed to the composition of each luminescent dye shown in Table I.

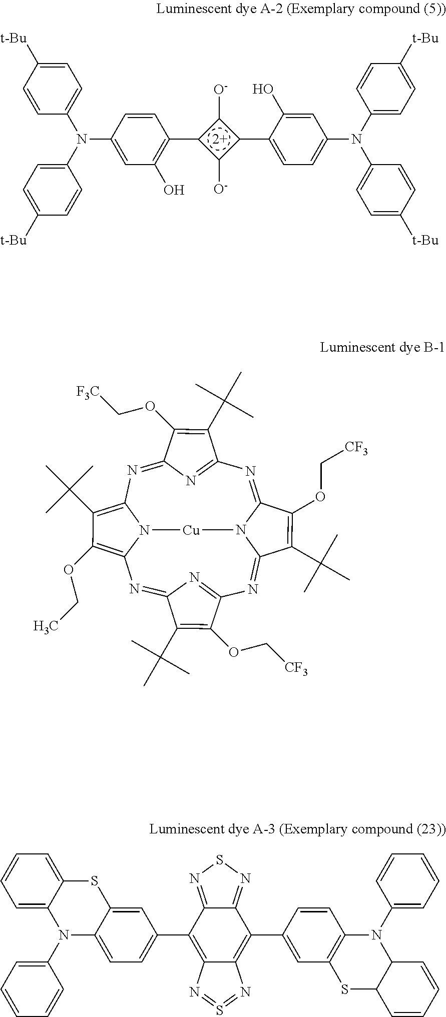

### [Preparation of a Wavelength Conversion Film 1-8]

### (Preparation of Titanium Oxide Dispersion)

A titanium oxide dispersion was prepared according to the following method. Titanium oxide (R-42, manufactured by Sakai Chemical Co., Ltd.) and propylene glycol monomethyl ether were mixed at a mass ratio of 50:50 and stirred with a stirrer to prepare a titanium oxide dispersion.

### (Preparation of a Wavelength Conversion Film 1-8)

A wavelength conversion film 1-8 having the configuration shown in FIG. 2 was produced according to the following method. A matrix material polystyrene (manufactured by ACROS ORGANICS Co., Ltd., weight average molecular weight Mw=260000) and a luminescent dye (exemplary compound Da-45) were added to toluene as a solvent in a mass ratio of 98.5:0.5. These were placed in an eggplant flask and heated and stirred at 80° C. to sufficiently dissolve them. Further, the titanium oxide dispersion prepared above was added to the prepared solution so that (polystyrene+luminescent dye):(titanium oxide) had a mass ratio of 99:1, and these were further stirred to prepare a mixed solution.

Next, the obtained mixed solution was applied onto a gas barrier film (4A) similar to that used as the base material of the organic EL element described above using an applicator, dried at room temperature for 10 minutes. Then further, it was heated and dried at 80° C. for 10 minutes to obtain a luminescent dye layer 5. Then, a gas barrier film 4B to which the sealing adhesive 6 was attached was prepared and bonded to the coated surface, and sealed using a vacuum laminator under heating conditions of 90° C. Then, the adhesive was cured by heat treatment at 110° C. for 30 minutes to prepare a wavelength conversion film 1-8 having a film thickness of 80 μm.

### <<Preparation of Light Emitting Members>>

### [Preparation of a Light Emitting Member 1-1]

By bringing the light emitting surface of the organic EL element A, which was the prepared surface light source, into close contact with the wavelength conversion film 1-1, the light emitting member 1-1 having the configuration shown in FIG. 1 was prepared.

### [Preparation of Light Emitting Members 1-2 to 1-4 and 1-8 to 1-11]

Light emitting members 1-2 to 1-4 and 1-8 to 1-11 were prepared in the same manner as preparation of the light emitting member 1-1, except that the wavelength conversion films 1-2 to 1-8 were used instead of the wavelength conversion film 1-1.

### [Preparation of a Light Emitting Member 1-5]

An element composed of only the organic EL element A produced above without providing a wavelength conversion film was designated as a light emitting member 1-5.

### [Preparation of a Light Emitting Member 1-6]

An element composed of only the organic EL element B produced above without providing a wavelength conversion film was designated as a light emitting member 1-6.

### [Preparation of a Light Emitting Member 1-7]

An element composed of only the organic EL element C produced above without providing a wavelength conversion film was designated as a light emitting member 1-7.

### <<Evaluation of Light Emitting Members>>

[Evaluation of Maximum Emission Wavelength (λₘₐₓ)]

Each of the light emitting members produced above was made to emit light, and the emission spectrum was measured using Fluorescence Spectrophotometer F-7000 (manufactured by Hitachi High-Tech Corporation). The wavelength of the peak top of the emission obtained on the longest wave side was defined as the emission maximum wavelength (λₘₐₓ).

Based on the measured maximum emission wavelength (λₘₐₓ), the maximum emission wavelength (λₘₐₓ) was evaluated according to the following criteria.

AA: The maximum emission wavelength (λₘₐₓ) is 780 nm or more.

BB: The maximum emission wavelength (λₘₐₓ) is 700 nm or more and less than 780 nm.

CC: The maximum emission wavelength (λₘₐₓ) is less than 700 nm.

### [Measurement of Emission Intensity (Relative Value)

Each of the manufactured light emitting members was made to emit light, and the light emission spectrum was measured using the above Fluorescence Spectrophotometer F-7000 (manufactured by Hitachi High-Tech Corporation). Then, the emission intensity at an emission wavelength of 850 nm of each light emitting member was determined. The relative value when the light emitting intensity of the light emitting member 1-7 was set to 100 was obtained.

### [Evaluation of Light Emission Life (LT80)]

Each light emitting member was continuously emitted at room temperature (25° C.) under a constant current condition of 50 mA/cm². The time (LT80) at which it became 80% of the initial brightness was measured, and the emission life was evaluated according to the following criteria.

AA: The light emission life (LT80) is 30 days or more.

BB: The light emission life (LT80) is 7 days or more and less than 30 days.

CC: The light emission life (LT80) is less than 7 days.

The results obtained from the above are shown in Table I.

TABLE I

| | | | | | Each evaluation result | | | |
|---|---|---|---|---|---|---|---|---|
| Light | Surface light source: | | | | Maximum | Emission | | |
| emitting | Organic EL element | | Wavelength conversion film | | emission | intensity | Emission | |
| member | | Luminescent | | Luminescent | wavelength | (Relative | life | |
| number | Number | dye | Number | dye | (λₘₐₓ) | value) | (LT80) | Remarks |
| 1-1 | A | — | 1-1 | A-1 | BB | 120 | AA | Present Invention |
| 1-2 | A | — | 1-2 | A-1 + B-1 | BB | 140 | AA | Present Invention |
| 1-3 | A | — | 1-3 | A-1 + A-3 | BB | 150 | AA | Present Invention |
| 1-4 | A | — | 1-4 | A-1 + A-2 | AA | 170 | AA | Present Invention |
| 1-5 | A | — | — | — | CC | 0 | AA | Comparative Example |
| 1-6 | B | — | — | — | BB | 110 | BB | Comparative Example |
| 1-7 | C | A-1 | — | — | BB | 100 | CC | Comparative Example |
| 1-8 | A | — | 1-5 | Da-51 | BB | 120 | AA | Present Invention |
| 1-9 | A | — | 1-6 | Da-21 | AA | 170 | AA | Present Invention |
| 1-10 | A | — | 1-7 | Da-45 | AA | 140 | AA | Present Invention |
| 1-11 | A | — | 1-8 | Da-45 + TiO₂ | AA | 200 | AA | Present Invention |

As is clear from the results shown in Table I, in the light emitting members 1-1 to 1-4 and 1-8 to 1-11 having the configuration specified in the present invention, in the case of a film containing a luminescent dye that absorbs red light emitted from a surface light source and converts it into near-infrared light by a wavelength conversion film, it can be confirmed that light is obtained on the longer wave side of the near-infrared light region suitable for a vein recognition device.

Among them, in the light emitting member 1-4 composed of two kinds of squarylium compounds as the luminescent dye of the wavelength conversion film, the light emitting member 1-9 composed of one kind of squarylium compound, the light emitting member 1-11 composed of one kind of squarylium compound and titanium oxide particles, it can be seen that the effect is remarkable. On the other hand, the light emitting member 1-5 without the wavelength conversion film has a short emission maximum wavelength of less than 700 nm. The light emitting member 1-6 composed of the organic EL element B has insufficient light emitting intensity with respect to the light emitting member of the present invention.

The light emitting member 1-7 in which a light emitting dye is added to a red phosphorescent light emitting layer (EML) being an organic functional layer of an organic EL element and a surface light source was found to have a shorter light emission life than that of the present invention. When the luminescent dye according to the present invention is added to the EML, it is presumed that when the organic EL element is driven, it acts as a quenching material for luminescence. However, it is confirmed that by arranging the surface light source and the wavelength conversion film independently as in the present invention, a long-life light emitting member can be obtained without hindering the light emission of the surface light source by the luminescent dye.

### Example 2

(Preparation of a Wristband Type Electronic Device Equipped with Biometric Authentication Device)

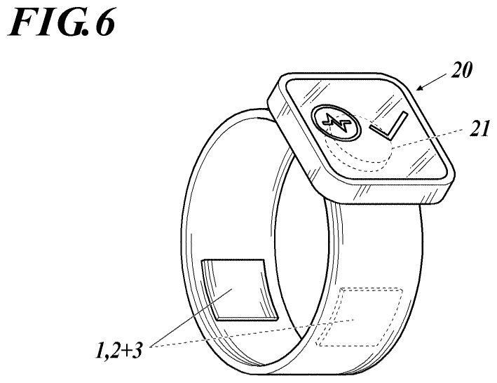

A wristband type electronic device 20 equipped with a biometric authentication function by imaging the wrist vein was produced. As shown in FIG. 6, this electronic device 20 contains an imaging unit 21 composed of a camera and a wide-angle lens placed on the outside of the wrist, and two light emitting members 1-4 prepared in Example 1 provided on a wristband other than on the same plane as the imaging unit when attached.

(Imaging with a Wristband Type Electronic Device Equipped with a Biometric Authentication Device)

When the wristband type electronic device 20 shown in FIG. 6 was attached on the wrist, it was possible to take an image of a clear vein pattern peculiar to the individual.

### Example 3

### (Preparation of a Biometric Authentication Device Using Vein Imaging of a Fingertip)

As shown in FIG. 3B, the surface light source 15 and the sensor (imaging unit) 12 were arranged so as to face each other with the finger 13A sandwiched between them, and a device capable of biometric authentication by vein imaging of the fingertip was produced.

(Imaging with a Biometric Authentication Device Using Vein Imaging of a Fingertip)

By performing vein imaging of a fingertip using the biometric authentication device shown in FIG. 3B, it was possible to take an image of the vein pattern peculiar to the individual.

### Example 4

### [Preparation of a Wavelength Conversion Film 2-1]

A wavelength conversion film 2-1 was prepared in the same manner as preparation of the wavelength conversion film 1-8 according to the first embodiment, except that, as shown in FIG. 13, the red light cut filter 7 was further provided between the luminescent dye layer 5 and the adhesive layer 6.

### [Preparation of a Light Emitting Member 2-1]

By bringing the light emitting surface of the organic EL element A, which is the surface light source produced in Example 1, into close contact with the wavelength conversion film 2-1 produced above, the light emitting member 2-1 having the configuration shown in FIG. 1 was produced. It was confirmed that the produced light emitting member 2-1 is a light emitting member of the present invention having a maximum emission wavelength of 700 nm or more that does not emit red light of the organic EL element A.

### Example 5

### (Preparation of a Pulse Oximeter)

A pulse oximeter was produced by arranging the red light emitting OLED panel and the light emitting member 2-1 (arranged at the position shown by 15) produced in Example 4 in such a manner that the light from the light source entering and scattered inside of the wrist was received at a sensor 12 as shown in FIG. 5B.

(Measurement of Oxygen Saturation with a Pulse Oximeter)

It was possible to measure an oxygen saturation with the prepared pulse oximeter attached to the wrist.

### Example 6

### (Preparation of a Transmissive Pulse Wave Sensor at a Fingertip Using a Surface Light Source)

A surface light source 15 using the light emitting member 2-1 produced in Example 4 and a sensor 12 were arranged as shown in FIG. 3B to produce a transmissive pulse wave sensor that enables to perform measurement at a fingertip.

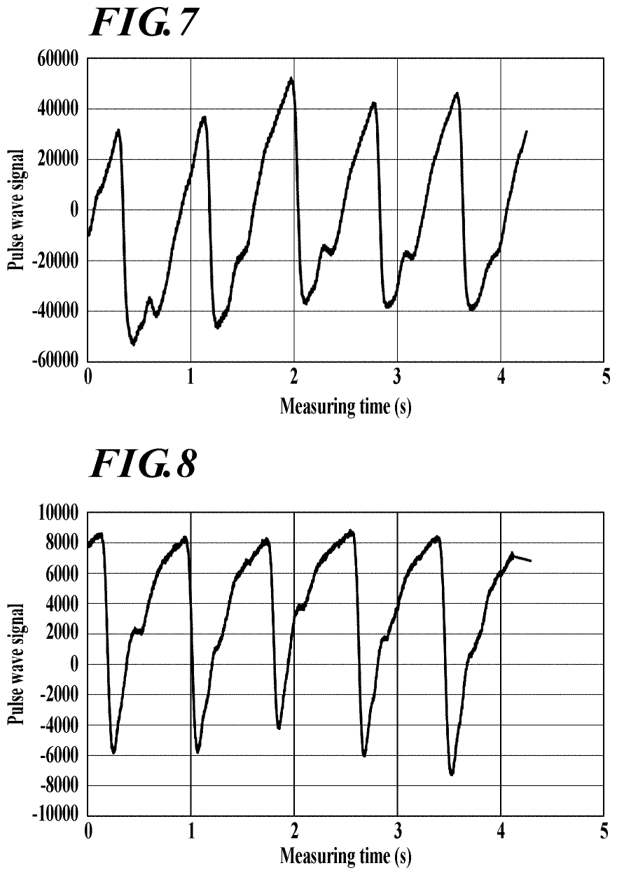

The transmissive pulse wave sensor produced above was attached to the index fingertip 13A, and the pulse wave was measured at a fingertip. As a result, as shown in FIG. 7, it was possible to obtain a strong pulse wave signal.

### Example 7

### (Preparation of a Reflective Pulse Wave Sensor at a Fingertip Using a Surface Light Source)

A surface light source 15 using the light emitting member 2-1 produced in Example 4 and a sensor 12 were arranged as shown in FIG. 3C to produce a reflective pulse wave sensor that enables to perform measurement at a fingertip.

FIG. 3C is a schematic view showing reflective sensing at a fingertip using the surface light source produced above.

The reflective pulse wave sensor produced above was attached to a thumb tip 13A, and the pulse wave was measured at the fingertip. As a result, it was possible to obtain a strong pulse wave signal as shown in FIG. 8.

### Example 8

### (Preparation of a Transmissive Pulse Wave Sensor at a Base of a Finger Using a Surface Light Source)

A surface light source 15 using the light emitting member 2-1 produced in Example 4 and a sensor 12 were arranged as shown in FIG. 4B to produce a transmissive pulse wave sensor that enables to perform measurement at a base of a finger.

FIG. 4B is a schematic view showing transmissive sensing at a base of a finger using the surface light source produced above.

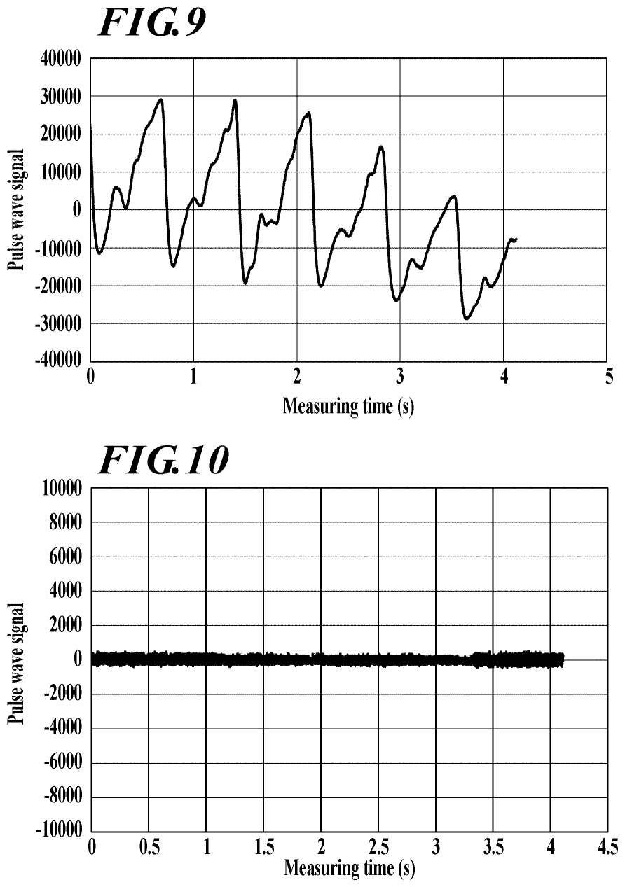

The transmissive pulse wave sensor produced above was attached to a base of a thumb 13B, and the pulse wave was measured at the base of the finger. As a result, it was possible to obtain a strong pulse wave signal as shown in FIG. 9.

### Comparative Example 2

### (Preparation of a Transmissive Pulse Wave Sensor at a Base of a Finger Using a Point Light Source)

A transmissive pulse wave sensor was prepared by arranging an LED (maximum emission wavelength 850 nm) that emits near-infrared light as a point light source 11 and a sensor 12 as shown in FIG. 4A.

FIG. 4A is a schematic view showing transmissive sensing at a base of a finger using the point light source produced above. It is the same as Example 8 except that a point light source is used.

The transmissive pulse wave sensor produced above was attached to a base of a thumb 13B, and the pulse wave was measured at the base of the finger. As a result, it was possible to obtain a pulse wave signal as shown in FIG. 10.

In comparing the results of Example 8 using the surface light source and Comparative Example 2 using the point light source, a strong pulse wave signal was obtained only in Example 8. It is clear that the cause of this is the difference in the shape of the light source, that is, the use of the surface light source in Example 8 was effective. By newly applying the surface light source according to the present invention to pulse wave measurement, it has become possible to measure pulse waves at a base of a finger, which was difficult to measure with a point light source of the prior art.

### Example 9

### (Preparation of a Reflective Pulse Wave Sensor on a Wrist Using a Surface Light Source)

As shown in FIG. 5C, a surface light source 15 using the light emitting member 2-1 produced in Example 4 and a sensor 12 were arranged to produce a reflective pulse wave sensor that enables to perform measurement on a wrist.

FIG. 5C is a schematic view showing reflective sensing on a wrist using the surface light source produced above.

The reflective pulse wave sensor produced above was attached to a wrist 16 and the pulse wave was measured on the wrist. As a result, as shown in FIG. 11, it was possible to obtain a strong pulse wave signal.

### Comparative Example 3

### (Preparation of a Reflective Pulse Wave Sensor on a Wrist Using a Point Light Source)

As shown in FIG. 5D, a reflective pulse wave sensor was produced by arranging an LED (maximum emission wavelength 850 nm) that emits near-infrared light as a point light source 11 and a sensor 12.

FIG. 5D is a schematic view showing transmissive sensing on a wrist using the point light source produced above. It is the same as Example 9 except that a point light source is used.

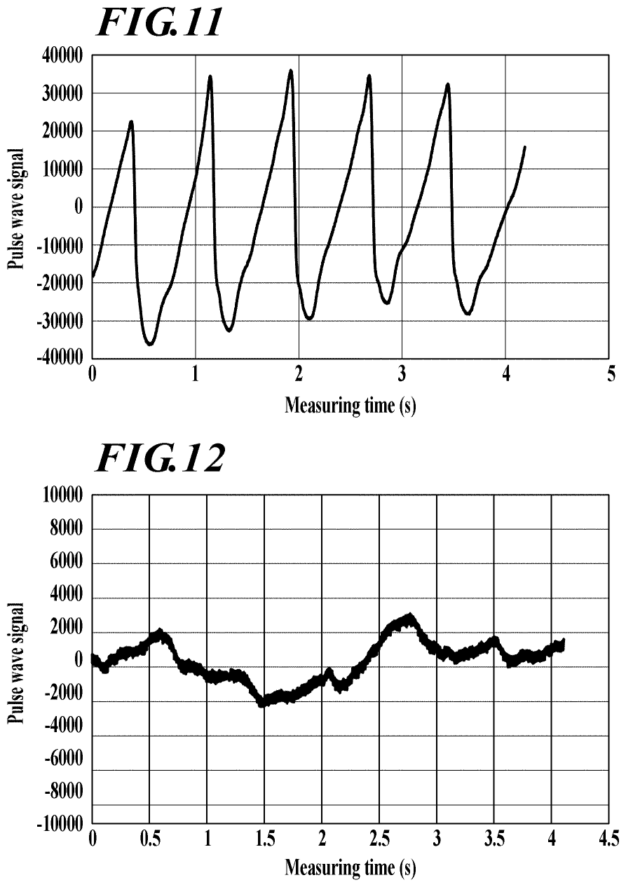

The reflective pulse wave sensor produced above was attached to the wrist 16 and the pulse wave was measured on the wrist. As a result, as shown in FIG. 12, almost no pulse wave signal was obtained.

In comparing the results of Example 9 using the surface light source and Comparative Example 3 using the point light source, a strong pulse wave signal was obtained only in Example 9. It is clear that the cause of this is the difference in the shape of the light source, that is, the use of the surface light source in Example 9 was effective. By newly applying the surface light source according to the present invention to pulse wave measurement, it has become possible to measure pulse waves on the wrist, which was difficult to measure with a point light source of the prior art.

### INDUSTRIAL APPLICABILITY

The light emitting member of the present invention has a maximum emission wavelength in the near-infrared light region, is excellent in emission intensity and emission life. It is suitably used for a biometric authentication device using vein authentication, a wristband type electronic device, and a biometric device.

### DESCRIPTION OF SYMBOLS

1: Light emitting member
2: Surface light source
3: Wavelength conversion film
4A, 4B: Gas barrier film
5: Luminescent dye layer
6: Adhesive layer
7: Red light cut filter
11: Point light source
12: Sensor
13A: Fingertip
13B: Base of a finger
14: Bone
15: Surface light source
16: Wrist
20: Wristband type electronic device
21: Imaging unit
IR: Near-infrared light
L: Light
R: Red light emission

1. A light emitting member that emits near-infrared light, comprising a surface light source that emits at least red light and a wavelength conversion film that converts visible light of the surface light source into near-infrared light, wherein the light emitting member has a maximum emission wavelength in the wavelength range of 700 to 1500 nm.

2. The light emitting member described in claim 1, wherein the wavelength conversion film contains a luminescent dye.

3. The light emitting member described in claim 2, wherein the luminescent dye is a squarylium compound, a croconium compound, or a thiadiazole compound.

4. The light emitting member described in claim 3, wherein the squarylium compound is a compound having a structure represented by the following Formula (1), 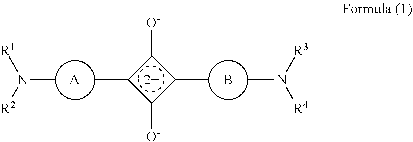
in Formula (1), A and B respectively represent an aromatic hydrocarbon ring which may have a substituent, or an aromatic heterocycle which may have a substituent, and adjacent substituents may be bonded to each other to form a ring; and R¹ to R⁴ respectively represent a substituent, at least one of R¹ to R⁴ has an aromatic hydrocarbon ring, and R¹ to R⁴ may be respectively bonded to each other to form a ring.

5. The light emitting member described in claim 4, wherein the squarylium compound having a structure represented by Formula (1) is a compound having a structure represented by the following Formula (2), 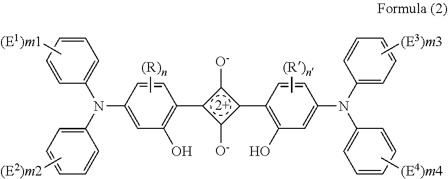
In Formula (2), E¹ to E⁴ respectively represent a substituent; m1 to m4 respectively represent 0 or an integer of 1 to 5; R and R′ respectively represent a substituent; and n and n′ respectively represent 0 or an integer of 1 to 3, provided that when m1 to m4, n and n′ are 2 or more, E¹ to E⁴, R and R′ may be the same or different.

6. The light emitting member described in claim 2, wherein the wavelength conversion film contains two or more luminescent dyes.

7. The light emitting member described in claim 2, wherein the wavelength conversion film contains two or more squarylium dyes.

8. The light emitting member described in claim 1, wherein the surface light source is an organic electroluminescent element.

9. The light emitting member described in claim 1, wherein the maximum emission wavelength obtained from the light emitting member is 780 nm or more.

10. A biometric authentication device equipped with the light emitting member described in claim 1.

11. A wristband electronic device equipped with the biometric authentication device described in claim 10 so as to carry out biometric authentication by taking an image of a wrist vein.

12. The wristband electronic device described in claim 11, wherein when the wristband electronic device is mounted to a wrist, a light source is provided in any area on a wristband of the wristband electronic device other than on the same plane as an imaging unit.

13. A biometric device equipped with the light emitting member described in claim 1.

14. The biometric device described in claim 13, being a pulse oximeter that performs measurement at a wrist or a base of a finger.

15. The biometric device described in claim 13, being a pulse wave sensor that performs measurement at a wrist or a base of a finger.
